SEQUENCE LISTING (179 sequences)

SEQ ID NO: 1 - RNA, 23 nt, Homo sapiens
uggcagugucuuagcugguuguu

SEQ ID NO: 2 - RNA, 18 nt, Artificial Sequence
  Description of Artificial Sequence: Synthetic oligonucleotide
ggcaguguuuagcuguug

SEQ ID NO: 3 - RNA, 21 nt, Homo sapiens
ucguaccgugaguaauaaugc

SEQ ID NO: 4 - RNA, 22 nt, Homo sapiens
uuaaggcacgcggugaaugcca

SEQ ID NO: 5 - RNA, 20 nt, Homo sapiens
guguguggaaaugcuucugc

SEQ ID NO: 6 - RNA, 21 nt, Homo sapiens
augaccuaugaauugacagac

SEQ ID NO: 7 - RNA, 21 nt, Homo sapiens
uacaguacugugauaacugaa

SEQ ID NO: 8 - RNA, 21 nt, Homo sapiens
gugccagcugcagugggggag

SEQ ID NO: 9 - RNA, 21 nt, Homo sapiens
aucacauugccagggauuucc

SEQ ID NO: 10 - RNA, 22 nt, Homo sapiens
uagcagcacauaaugguuugug

SEQ ID NO: 11 - RNA, 22 nt, Homo sapiens
ugagguaguagauuguauaguu

SEQ ID NO: 12 - RNA, 22 nt, Homo sapiens
aagcugccaguugaagaacugu

SEQ ID NO: 13 - RNA, 22 nt, Homo sapiens
ugcuaugccaacauauugccau

SEQ ID NO: 14 - RNA, 22 nt, Homo sapiens
uggcucaguucagcaggaacag

SEQ ID NO: 15 - RNA, 21 nt, Homo sapiens
uucacaguggcuaaguuccgc

SEQ ID NO: 16 - RNA, 21 nt, Homo sapiens
uucacaguggcuaaguucugc

SEQ ID NO: 17 - RNA, 22 nt, Homo sapiens
ugagguaguagguuguauaguu

SEQ ID NO: 18 - RNA, 22 nt, Homo sapiens
ugagguaguagguugugugguu

SEQ ID NO: 19 - RNA, 22 nt, Homo sapiens
guggguacggcccagugggggg

SEQ ID NO: 20 - RNA, 25 nt, Homo sapiens
agggaucgcgggcggguggcggccu

SEQ ID NO: 21 - RNA, 21 nt, Homo sapiens
aucacauugccagggauuacc

SEQ ID NO: 22 - RNA, 22 nt, Homo sapiens
ugagguaguaguuugugcuguu

SEQ ID NO: 23 - RNA, 22 nt, Homo sapiens
agagguaguagguugcauaguu

SEQ ID NO: 24 - RNA, 22 nt, Homo sapiens
ugagguaggagguuguauaguu

SEQ ID NO: 25 - RNA, 22 nt, Homo sapiens
uagcagcacguaaauauuggcg

SEQ ID NO: 26 - RNA, 21 nt, Homo sapiens
aggcaagaugcuggcauagcu

SEQ ID NO: 27 - RNA, 18 nt, Homo sapiens
gcaugggugguucagugg

SEQ ID NO: 28 - RNA, 21 nt, Homo sapiens
caacaccagucgaugggcugu

SEQ ID NO: 29 - RNA, 23 nt, Homo sapiens
uaaagugcuuauagugcagguag

SEQ ID NO: 30 - RNA, 22 nt, Homo sapiens
uuuucaacucuaaugggagaga

SEQ ID NO: 31 - RNA, 18 nt, Homo sapiens
aucccaccucugccacca

SEQ ID NO: 32 - RNA, 21 nt, Homo sapiens
gccccugggccuauccuagaa

SEQ ID NO: 33 - RNA, 22 nt, Homo sapiens
uagcuuaucagacugauguuga

SEQ ID NO: 34 - RNA, 21 nt, Homo sapiens
cgcgggugcuuacugacccuu

SEQ ID NO: 35 - RNA, 22 nt, Homo sapiens
uguaaacauccuacacucagcu

SEQ ID NO: 36 - RNA, 22 nt, Homo sapiens
uaacacugucugguaaagaugg

SEQ ID NO: 37 - RNA, 22 nt, Homo sapiens
uaauacugccugguaaugauga

SEQ ID NO: 38 - RNA, 22 nt, Homo sapiens
uagcagcacaucaugguuuaca

SEQ ID NO: 39 - RNA, 22 nt, Homo sapiens
aaggagcucacagucuauugag

SEQ ID NO: 40 - RNA, 21 nt, Homo sapiens
uggacugcccugaucuggaga

SEQ ID NO: 41 - RNA, 23 nt, Homo sapiens
ugugcaaauccaugcaaaacuga

SEQ ID NO: 42 - RNA, 23 nt, Homo sapiens
agcagcauuguacagggcuauca

SEQ ID NO: 43 - RNA, 23 nt, Homo sapiens
caaagugcuuacagugcagguag

SEQ ID NO: 44 - RNA, 22 nt, Homo sapiens
ugagguaguaguuuguacaguu

SEQ ID NO: 45 - RNA, 17 nt, Homo sapiens
ucccaccgcugccaccc

SEQ ID NO: 46 - RNA, 20 nt, Homo sapiens
acugccccaggugcugcugg

SEQ ID NO: 47 - RNA, 18 nt, Homo sapiens
gucccuguucaggcgcca

SEQ ID NO: 48 - RNA, 21 nt, Homo sapiens
ucgaggagcucacagucuagu

SEQ ID NO: 49 - RNA, 22 nt, Homo sapiens
aaaagcuggguugagagggcga

SEQ ID NO: 50 - RNA, 17 nt, Homo sapiens
ucucgcuggggccucca

SEQ ID NO: 51 - RNA, 21 nt, Homo sapiens
caagucacuagugguuccguu

SEQ ID NO: 52 - RNA, 22 nt, Homo sapiens
ugagguaguagguuguaugguu

SEQ ID NO: 53 - RNA, 23 nt, Homo sapiens
ugugcaaaucuaugcaaaacuga

SEQ ID NO: 54 - RNA, 22 nt, Homo sapiens
ggaggggucccgcacugggagg

SEQ ID NO: 55 - RNA, 17 nt, Homo sapiens
ucccuguucgggcgcca

SEQ ID NO: 56 - RNA, 22 nt, Homo sapiens
aaaagcuggguugagagggcaa

SEQ ID NO: 57 - RNA, 18 nt, Homo sapiens
cgggcguggugguggggg

SEQ ID NO: 58 - RNA, 23 nt, Homo sapiens
agcagcauuguacagggcuauga

SEQ ID NO: 59 - RNA, 22 nt, Homo sapiens
aacuggcccucaaagucccgcu

SEQ ID NO: 60 - RNA, 19 nt, Homo sapiens
gagccaguuggacaggagc

SEQ ID NO: 61 - RNA, 23 nt, Homo sapiens
uaauacugccggguaaugaugga

SEQ ID NO: 62 - RNA, 22 nt, Homo sapiens
uguaaacauccccgacuggaag

SEQ ID NO: 63 - RNA, 22 nt, Homo sapiens
cagugcaaugaugaaagggcau

SEQ ID NO: 64 - RNA, 22 nt, Homo sapiens
aggcggggcgccgcgggaccgc

SEQ ID NO: 65 - RNA, 23 nt, Homo sapiens
uagcagcgggaacaguucugcag

SEQ ID NO: 66 - RNA, 23 nt, Homo sapiens
uagcaccauuugaaaucaguguu

SEQ ID NO: 67 - RNA, 22 nt, Homo sapiens
cugugcgugugacagcggcuga

SEQ ID NO: 68 - RNA, 22 nt, Homo sapiens
uagguaguuucauguuguuggg

SEQ ID NO: 69 - RNA, 20 nt, Homo sapiens
ccccagggcgacgcggcggg

SEQ ID NO: 70 - RNA, 22 nt, Homo sapiens
uagcaccaucugaaaucgguua

SEQ ID NO: 71 - RNA, 22 nt, Homo sapiens
uauugcacuugucccggccugu

SEQ ID NO: 72 - RNA, 19 nt, Homo sapiens
aaaagcuggguugagagga

SEQ ID NO: 73 - RNA, 23 nt, Homo sapiens
cgcauccccuagggcauuggugu

SEQ ID NO: 74 - RNA, 21 nt, Homo sapiens
uaacagucuccagucacggcc

SEQ ID NO: 75 - RNA, 22 nt, Homo sapiens
uauggcacugguagaauucacu

SEQ ID NO: 76 - RNA, 22 nt, Homo sapiens
aacccguagauccgaacuugug

SEQ ID NO: 77 - RNA, 23 nt, Homo sapiens
aacauucaacgcugucggugagu

SEQ ID NO: 78 - RNA, 23 nt, Homo sapiens
uuaaugcuaaucgugauaggggu

SEQ ID NO: 79 - RNA, 22 nt, Homo sapiens
ucccugagacccuaacuuguga

SEQ ID NO: 80 - RNA, 22 nt, Homo sapiens
uuaucagaaucuccagggguac

SEQ ID NO: 81 - RNA, 22 nt, Homo sapiens
uguaaacauccucgacuggaag

SEQ ID NO: 82 - RNA, 22 nt, Homo sapiens
uagcaccauuugaaaucgguua

SEQ ID NO: 83 - RNA, 23 nt, Homo sapiens
uuuggcacuagcacauuuuugcu

SEQ ID NO: 84 - RNA, 22 nt, Homo sapiens
uggcagugucuuagcugguugu

SEQ ID NO: 85 - RNA, 22 nt, Homo sapiens
aauccuuugucccugggugaga

SEQ ID NO: 86 - RNA, 22 nt, Homo sapiens
cacuggcuccuuucuggguaga

SEQ ID NO: 87 - RNA, 24 nt, Homo sapiens
ucccugagacccuuuaaccuguga

SEQ ID NO: 88 - RNA, 22 nt, Homo sapiens
ugaaacauacacgggaaaccuc

SEQ ID NO: 89 - RNA, 23 nt, Homo sapiens
uaauccuugcuaccugggugaga

SEQ ID NO: 90 - RNA, 22 nt, Homo sapiens
uucaccaccuucuccacccagc

SEQ ID NO: 91 - RNA, 24 nt, Homo sapiens
uggggagcugaggcucugggggug

SEQ ID NO: 92 - RNA, 23 nt, Homo sapiens
aacauucauugcugucggugggu

SEQ ID NO: 93 - RNA, 21 nt, Homo sapiens
aaggcagggcccccgcucccc

SEQ ID NO: 94 - RNA, 23 nt, Homo sapiens
ucucacacagaaaucgcacccgu

SEQ ID NO: 95 - RNA, 23 nt, Homo sapiens
agcuacauugucugcuggguuuc

SEQ ID NO: 96 - RNA, 20 nt, Homo sapiens
aaaagcuggguugagagggu

SEQ ID NO: 97 - skipped

SEQ ID NO: 98 - RNA, 19 nt, Homo sapiens
gugaggacucgggaggugg

SEQ ID NO: 99 - RNA, 23 nt, Homo sapiens
caaagugcuguucgugcagguag

SEQ ID NO: 100 - RNA, 22 nt, Homo sapiens
cagcagcaauucauguuuugaa

SEQ ID NO: 101 - RNA, 23 nt, Homo sapiens
uggaagacuagugauuuuguugu

SEQ ID NO: 102 - RNA, 21 nt, Homo sapiens
uaaagugcugacagugcagau

SEQ ID NO: 103 - RNA, 23 nt, Homo sapiens
caaagugcucauagugcagguag

SEQ ID NO: 104 - RNA, 22 nt, Homo sapiens
acaggugagguucuugggagcc

SEQ ID NO: 105 - RNA, 21 nt, Homo sapiens
uggcagggaggcugggagggg

SEQ ID NO: 106 - RNA, 22 nt, Homo sapiens
uccuucauuccaccggagucug

SEQ ID NO: 107 - RNA, 22 nt, Homo sapiens
cagugcaauguuaaaagggcau

SEQ ID NO: 108 - RNA, 22 nt, Homo sapiens
auauaauacaaccugcuaagug

SEQ ID NO: 109 - RNA, 22 nt, Homo sapiens
cauugcacuugucucggucuga

SEQ ID NO: 110 - RNA, 22 nt, Homo sapiens
uagguaguuuccuguuguuggg

SEQ ID NO: 111 - RNA, 22 nt, Homo sapiens
ugugacugguugaccagagggg

SEQ ID NO: 112 - RNA, 22 nt, Homo sapiens
uuauaauacaaccugauaagug

SEQ ID NO: 113 - RNA, 23 nt, Homo sapiens
uacccuguagauccgaauuugug

SEQ ID NO: 114 - RNA, 23 nt, Homo sapiens
uguaaacauccuacacucucagc

SEQ ID NO: 115 - RNA, 22 nt, Homo sapiens
ugagguaguaaguuguauuguu

SEQ ID NO: 116 - RNA, 23 nt, Homo sapiens
aaugacacgaucacucccguuga

SEQ ID NO: 117 - RNA, 22 nt, Homo sapiens
aacuggccuacaaagucccagu

SEQ ID NO: 118 - RNA, 22 nt, Homo sapiens
ucggccugaccacccaccccac

SEQ ID NO: 119 - RNA, 23 nt, Homo sapiens
uaaggugcaucuagugcagauag

SEQ ID NO: 120 - RNA, 21 nt, Homo sapiens
cuagacugaagcuccuugagg

SEQ ID NO: 121 - RNA, 21 nt, Homo sapiens
caucccuugcaugguggaggg

SEQ ID NO: 122 - RNA, 22 nt, Homo sapiens
aaucguacagggucauccacuu

SEQ ID NO: 123 - RNA, 22 nt, Homo sapiens
aacccguagauccgaucuugug

SEQ ID NO: 124 - RNA, 22 nt, Homo sapiens
uaaugccccuaaaaauccuuau

SEQ ID NO: 125 - RNA, 24 nt, Homo sapiens
gcugguuucauauggugguuuaga

SEQ ID NO: 126 - RNA, 23 nt, Homo sapiens
ugagugugugugugugagugugu

SEQ ID NO: 127 - RNA, 22 nt, Homo sapiens
gagcuuauucauaaaagugcag

SEQ ID NO: 128 - RNA, 22 nt, Homo sapiens
uggagagaaaggcaguuccuga

SEQ ID NO: 129 - RNA, 23 nt, Homo sapiens
agguugggaucgguugcaaugcu

SEQ ID NO: 130 - RNA, 21 nt, Homo sapiens
cugaccuaugaauugacagcc

SEQ ID NO: 131 - RNA, 22 nt, Homo sapiens
uguaacagcaacuccaugugga

SEQ ID NO: 132 - RNA, 22 nt, Homo sapiens
uaacacugucugguaacgaugu

SEQ ID NO: 133 - RNA, 22 nt, Homo sapiens
uucaaguaauccaggauaggcu

SEQ ID NO: 134 - RNA, 22 nt, Homo sapiens
uaauacugucugguaaaaccgu

SEQ ID NO: 135 - RNA, 22 nt, Homo sapiens
cuuucagucggauguuugcagc

SEQ ID NO: 136 - RNA, 21 nt, Homo sapiens
augaccuaugaauugacagac

SEQ ID NO: 137 - RNA, 21 nt, Homo sapiens
agcuacaucuggcuacugggu

SEQ ID NO: 138 - RNA, 22 nt, Homo sapiens
cacccguagaaccgaccuugcg

SEQ ID NO: 139 - RNA, 24 nt, Homo sapiens
aauccuuggaaccuaggugugagu

SEQ ID NO: 140 - RNA, 21 nt, Homo sapiens
uucaaguaauucaggauaggu

SEQ ID NO: 141 - RNA, 22 nt, Homo sapiens
uguaaacauccuugacuggaag

SEQ ID NO: 142 - RNA, 23 nt, Homo sapiens
uauggcuuuucauuccuauguga

SEQ ID NO: 143 - RNA, 23 nt, Homo sapiens
cagugcaauaguauugucaaagc

SEQ ID NO: 144 - RNA, 20 nt, Homo sapiens
guccgcucggcgguggccca

SEQ ID NO: 145 - RNA, 22 nt, Homo sapiens
cugcccuggcccgagggaccga

SEQ ID NO: 146 - RNA, 20 nt, Homo sapiens
ucacaccugccucgcccccc

SEQ ID NO: 147 - RNA, 22 nt, Homo sapiens
cugguacaggccugggggacag

SEQ ID NO: 148 - RNA, 20 nt, Homo sapiens
acucaaacugugggggcacu

SEQ ID NO: 149 - RNA, 23 nt, Homo sapiens
cgggucggaguuagcucaagcgg

SEQ ID NO: 150 - RNA, 21 nt, Homo sapiens
ugacaagccugacgagagcgu

SEQ ID NO: 151 - RNA, 21 nt, Homo sapiens
ucacagugaaccggucucuuu

SEQ ID NO: 152 - RNA, 22 nt, Homo sapiens
caugccuugaguguaggaccgu

SEQ ID NO: 153 - RNA, 22 nt, Homo sapiens
ugggucuuugcgggcgagauga

SEQ ID NO: 154 - RNA, 21 nt, Homo sapiens
gcgacccauacuugguuucag

SEQ ID NO: 155 - RNA, 22 nt, Homo sapiens
uacccauugcauaucggaguug

SEQ ID NO: 156 - RNA, 22 nt, Homo sapiens
ugagaacugaauuccauggguu

SEQ ID NO: 157 - RNA, 21 nt, Homo sapiens
ccgucgccgccacccgagccg

SEQ ID NO: 158 - RNA, 22 nt, Homo sapiens
cgacauggacgugcagggggau

SEQ ID NO: 159 - RNA, 17 nt, Homo sapiens
gugggggagaggcuguc

SEQ ID NO: 160 - RNA, 19 nt, Homo sapiens
aauggauuuuuggagcagg

SEQ ID NO: 161 - RNA, 23 nt, Homo sapiens
ucggggaucaucaugucacgaga

SEQ ID NO: 162 - RNA, 22 nt, Homo sapiens
ucacaagucaggcucuugggac

SEQ ID NO: 163 - RNA, 22 nt, Homo sapiens
gugaacgggcgccaucccgagg

SEQ ID NO: 164 - RNA, 27 nt, Homo sapiens
cacuguaggugauggugagagugggca

SEQ ID NO: 165 - RNA, 22 nt, Homo sapiens
ugcuggaucagugguucgaguc

SEQ ID NO: 166 - RNA, 22 nt, Homo sapiens
uccuguacugagcugccccgag

SEQ ID NO: 167 - RNA, 22 nt, Homo sapiens
auaaagcuagauaaccgaaagu

SEQ ID NO: 168 - RNA, 23 nt, Homo sapiens
uaggcagugucauuagcugauug

SEQ ID NO: 169 - RNA, 23 nt, Homo sapiens
aggcaguguaguuagcugauugc

SEQ ID NO: 170 - RNA, 22 nt, Homo sapiens
uggcaguguauuguuagcuggu

SEQ ID NO: 171 - RNA, 22 nt, Homo sapiens
aggcaguguauuguuagcuggc

SEQ ID NO: 172 - RNA, 25 nt, Homo sapiens
uaggcaguguauugcuagcggcugu

SEQ ID NO: 173 - RNA, 19 nt, Homo sapiens
uggcaguguauuguagcgg

SEQ ID NO: 174 - RNA, 7 nt, Homo sapiens
ggcagug

SEQ ID NO: 175 - RNA, 7 nt, Artificial Sequence
  Description of Artificial Sequence: Synthetic oligonucleotide
uaaggca

SEQ ID NO: 176 - RNA, 7 nt, Artificial Sequence
  Description of Artificial Sequence: Synthetic oligonucleotide
cguaccg

SEQ ID NO: 177 - RNA, 7 nt, Artificial Sequence
  Description of Artificial Sequence: Synthetic oligonucleotide
ugugugg

SEQ ID NO: 178 - RNA, 21 nt, Homo sapiens
cugaccuaugaauugacagcc

SEQ ID NO: 179 - RNA, 7 nt, Homo sapiens
ugaccua

(19) United States
(12) Patent Application Publication
(10) Pub. No.: US 2017/0035797 A1
(43) Pub. Date: Feb. 9, 2017

(54) COMBINATION CANCER TREATMENTS UTILIZING SYNTHETIC OLIGONUCLEOTIDES AND EGFR-TKI INHIBITORS

(71) Applicant: Mirna Therapeutics, Inc., Austin, TX (US)

(72) Inventors: Andreas BADER, Austin, TX (US); Jane ZHAO, Lakeway, TX (US); Kevin KELNAR, Kyle, TX (US)

(21) Appl. No.: 15/298,887

(22) Filed: Oct. 20, 2016

Related U.S. Application Data
(63) Continuation of application No. 14/215,669, filed on Mar. 17, 2014.
Application No. 14/215,669 is a continuation-in-part of application No. 14/212,105, filed on Mar. 14, 2014, now abandoned.
(60) Provisional application No. 61/927,543, filed on Jan. 15, 2014.
(60) Provisional application No. 61/787,558, filed on Mar. 15, 2013.

(51) Int. Cl. A61K 31/713; A61K 31/517; A61K 31/5377

(52) U.S. Cl. CPC A61K 31/713; A61K 31/5377; A61K 31/517

(57) ABSTRACT

The disclosure provides methods and compositions for treating cancer cells, including cancer cells in a subject, whereby two or more therapeutic agents are used, one being an EGFR-TKI agent and the other being a synthetic oligonucleotide.

### RELATED APPLICATIONS

This application is a continuation of U.S. application Ser. No. 14/215,669, filed Mar. 17, 2014, which is a continuation-in-part of U.S. application Ser. No. 14/212,105, filed Mar. 14, 2014, which claims benefit of priority to U.S. Provisional Application No. 61/787,558, filed Mar. 15, 2013, and U.S. Ser. No. 61/927,543, filed Jan. 15, 2014, all of which are all incorporated herein by reference in their entireties.

### SEQUENCE LISTING

The instant application contains a Sequence Listing which has been submitted electronically in ASCII format and is hereby incorporated by reference in its entirety. Said ASCII copy, created on Oct. 18, 2016, is named “48436717302.txt” and is 26,429 bytes in size.

### FIELD OF THE INVENTION

This invention relates to cancer therapy, and more specifically, to combination cancer therapy utilizing synthetic oligonucleotides and EGFR-TKI inhibitors.

### BACKGROUND OF THE INVENTION

Lung cancer accounts for the most cancer-related deaths in both men and women. An estimated ˜220,000 new cases of lung cancer are expected in 2012, accounting for about 14% of all cancer diagnoses (Cancer Facts & Figures. 2012, Society). Lung cancer is the leading cause of cancer-related deaths totaling in an estimated 160,000 deaths in 2012 which equals about 28% of all cancer deaths. Lung cancers are divided into two major classes. Small cell lung cancer (SCLC) affects 20% of patients and non-small cell lung cancer (NSCLC) affects approximately 80%. NSCLC consists of three major types: adenocarcinoma, squamous cell carcinoma, and large cell carcinoma, with lung adenocarcinomas and squamous cell carcinomas accounting for the vast majority of all lung cancers (see, e.g., Forgacs et al., Pathol Oncol Res, 2001. 7(1):6-13; Sekido et al., Biochim Biophys Acta, 1998. 1378(1): F21-59). Treatments include surgery, radiation, therapy, chemotherapy, and targeted therapies. For localized NSCLC, surgery is usually the treatment of choice, and survival for most of these patients improves by giving chemotherapy after surgery. Targeted therapies are used depending on the cancer genotype or stage of disease and include bevacizumab (Avastin™, Genentech/Roche), a humanized monoclonal antibody targeting VEGF-A, erlotinib (Tarceva™, Genentech/Roche), an EGFR tyrosine kinase inhibitor (EGFR-TKI), and crizotinib (Xalkori™, Pfizer), an inhibitor of ALK (anaplastic lymphoma kinase) and ROS1 (c-ros oncogene, receptor tyrosine kinase). Crizotinib has been approved by the FDA to treat certain late-stage (locally advanced or metastatic) non-small cell lung cancers and is limited to those that express the mutated ALK gene. Bevacizumab has been first approved for use in first-line advanced non-squamous NSCLC in combination with carboplatin/paclitaxel chemotherapy. Since then, the National Comprehensive Cancer Network recommends bevacizumab as standard first-line treatment in combination with any platinum-based chemotherapy, followed by maintenance bevacizumab until disease progression (Sandler et al., N Engl J Med, 2006. 355(24): 2542-50).

Erlotinib received fast-track approval from the US Food and Drug Administration (FDA) for patients with NSCLC after failure of prior conventional chemotherapy regimen (Cohen et al., Oncologist, 2005. 10(7):461-6; Cohen et al., Oncologist, 2003. 8(4):303-6. It is a reversible inhibitor of the EGFR kinase, designed to act as competitive inhibitors of ATP-binding at the active site of the EGFR kinase (Sharma et al. Nat Rev Cancer, 2007. 7(3):169-81). Gefitinib is another EGFR-TKI agent used in countries outside the US. Although no direct comparative effectiveness trials exist that have compared gefitinib with erlotinib, the data suggest that there are no major therapeutic differences between them (Pao et al., Nat Rev Cancer, 2010. 10(11): 760-74). Early clinical trials using EGFR-TKIs were modestly encouraging with partial responses observed in approximately 10-20% of treated patients with NSCLC (Fukuoka et al. J Clin Oncol, 2003. 21(12):2237-46). A drug response occurred more frequently in females, never-smokers, patients of Asian ethnicity, and those diagnosed with adenocarcinoma or bronchioalveolar histology Fukuoka et al., J Clin Oncol, 2003. 21(12):2237-46; Bell et al., J Clin Oncol, 2005. 23(31):8081-92). Notably, both drugs extend overall patient survival benefit by only ˜2 months, they lose their efficacy due to primary or acquired, secondary resistance (Sharma, supra; Shepherd et al., N Engl J Med, 2005. 353(2):123-32).

The dissatisfactory response rate of gefitinib and erlotinib has triggered multiple studies to assess the genetic background of responsive vs. resistant patient populations. Retroactive analyses of clinical trials revealed that EGFR expression levels did not correlate with a response to gefitinib (Bell, supra). Instead, patients responding to the drugs frequently harbored activating mutations in the EGFR kinase domain (id.). However, less than 50% of patients with EGFR mutations developed a response, indicating the presence of additional factors that determine susceptibility to EGFR-TKIs. Primary resistance or secondary resistance has been associated with (1) K-RAS mutations that may co-exist with EGFR mutations despite the fact that K-RAS and EGFR mutations appeared to be predominantly mutually exclusive (Gazdar et al., Trends Mol Med, 2004. 10(10):481-6; Pao et al., PLoS Med, 2005. 2(1):e17); (2) amplification and overexpression of c-Met, a receptor tyrosine kinase that signals into the PI3K pathway, substituting for an inactivation of EGFR (Engelman et al., Science, 2007. 316(5827):039-43); (3) the acquisition of a second mutation in the catalytic domain of EGFR (usually T790M) (Pao et al. PLoS Med, 2005. 2(3):e73., (4) BRAF mutations (Pratilas et al., Cancer Res, 2008. 68(22):9375-83); (5) ALK translocations (Shaw et al., J Clin Oncol, 2009. 27(26):4247-53); (6) hepatocyte growth factor (HGF) overexpression, the ligand of the MET receptor (Yano et al., Cancer Res, 2008. 68(22):9479-87); (7) the presence of other EGFR mutations (small insertions or duplications in exon 20: D770 N771, ins NPG, ins SVQ, ins G and N771T) (Wu et al., Clin Cancer Res, 2008. 14(15): 4877-82); and (8) genetic lesions that affect signaling downstream of EGFR, including PIK3CA (Engelman et al., J Clin Invest, 2006. 116(10):2695-706; Kawano et al., Lung Cancer, 2006. 54(2):209-15), loss of PTEN (Sos et al., Cancer Res, 2009. 69(8):3256-61), IGF1R and KDM5A (Gong et al., PLoS One, 2009. 4(10) e7273; Sharma et al., Cell. 141(1):69-80). The T790M mutation is found in ˜50% of EGFR-mutant tumors with acquired resistance; KRAS mutations occur in 15-25% of all NSCLCs; and mutated BRAF and ALK translocations are found in 2-3% and 5% of NSCLCs, respectively (Pao et al., Nat Rev Cancer, 2010. 10(11):760-74). Hence, the percentage on NSCLC patients that is likely to respond to EGFR-TKI therapy is relatively small. Additional yet unidentified molecular determinants may exist, which mediate resistance to EGFR inhibitors.

The modest efficacy of erlotinib as single therapeutic agents calls for the combinatorial use of these EGFR-TKIs with other therapeutic regimes. The Phase III clinical trials TRIBUTE/TALENT trials, investigating the effect of erlotinib in combination with cisplatin/gemcitabine or carboplatin/paclitaxel, failed to demonstrate a survival benefit of the drug over the conventional chemotherapies alone (Sharma, supra; Herbst et al., J Clin Oncol, 2005. 23(25):5892-9 and Giaccone et al., J Clin Oncol, 2004. 22(5):777-84 and Herbst et al., J Clin Oncol, 2004. 22(5):785-94. Therefore, erlotinib is currently being tested in combination with other targeted small molecule inhibitors that show promising results in preclinical studies, such inhibitors against mTOR and MET (Pao, supra). Whether this strategy is efficacious in patients with EGFR-TKI resistance remains to be established. Available data suggest that resistant tumors arise from rare cells in untreated tumors already harboring mutations in resistance genes, and that these subpopulations are selected for over the course of TM treatment (id.). It is also possible that already untreated tumors display a heterogenic profile of EGFR-TKI resistant cells, suggesting that a single drug combination of targeted therapies will not be sufficient for effective treatment. Instead, the sequential use of several combinations might be necessary to eliminate resistant tumors that undergo a positive selection during the prior treatment.

Therefore, despite advances in the treatment of lung cancer, the survival rate of lung cancer patients remains extremely poor. Current targeted therapies, such as EGFR-TKIs, hold considerable promise but lack satisfactory efficacy in monotherapy due to the existence or development of primary and secondary resistance. The combined use of EGFR inhibitors with other targeted treatments may aid in the efficacy of EGFR inhibitors and may help overcome or prevent drug resistance.

Preliminary studies indicate that certain miRNAs can sensitize cancer cells in vitro (reviewed in Bommer et aL, Curr Biol, 2007. 17(15):1298-307). For instance, let-7 is able to sensitize lung cancer cells to TRAIL-based, gemcitabine or radiation therapies (Li et al., Cancer Res, 2009. 69(16): 6704-12; Ovcharenko et al., Cancer Res, 2007. 67(22): 10782-8; Weidhaas et al., Cancer Res, 2007. 67(23):11111-6) Similarly, miR-34 enhances the efficiency of conventional therapies in cancer cell lines of the prostate, colon, brain, stomach, bladder and pancreas (Fujita et al., Biochem Biophys Res Commun, 2008. 377(1):114-9; Ji et al., PLoS One, 2009. 4(8):e6816; Kojima et al., Prostate. 70(14):1501-12. Akao et al., Cancer Lett. 300(2):197-204; Weeraratne et al., Neuro Oncol. 13(2):165-75; Ji et al., BMC Cancer, 2008. 8:266; and Vinall et al., Int J Cancer, 2011. 130(11): 2526-38). However, a demonstration for any erlotinib/miRNA combination in cell and animal models of lung cancer remains absent.

Recently, Zong et al. (Chemico-Bio Interac. 2010, 184:431-438) have tested let-7a, miR-126 and miR-145 for their ability to sensitize Gefitinib-resistant cells lines A549 and H460 to gefitinib. The biggest reduction of IC₅₀ was achieved by miR-126 in H440 cells (˜7-fold), whereas the remaining conditions resulted in only 2-3-fold IC₅₀ reductions (see Table 2 in Zhong, supra).

### SUMMARY OF THE INVENTION

The invention is based, in part, on the discovery that certain microRNAs can be consistently up- or down-regulated in EGFR-TKI-resistant cell lines, and that specific combinations of synthetic oligonucleotides and EGFR-TKI agents can have advantageous and/or unexpected results, for example because they are particularly efficacious in treating certain cancer cells (e.g., synergize, or have greater that additive effect). Accordingly, the invention, in various aspects and embodiments includes contacting cells, tissue, and/or organisms with specific combinations of synthetic oligonucleotides and EGFR-TKI agents. More particularly, the invention can include contacting cancer cells, cancer tissue, and/or organisms having cancer with such combinations of synthetic oligonucleotides and EGFR-TKI agents. The methods can be experimental, diagnostic, and/or therapeutic. The methods can be used to inhibit, or reduce the proliferation of, cells, including cells in a tissue or an organism. The synthetic oligonucleotides can be, for example, mimics or inhibitors of microRNAs that are consistently down- or up-regulated in EGFR-TKI-resistant cells lines.

Accordingly, in various aspects and embodiments, the invention provides methods of treating a subject having a cancer. In certain embodiments, the methods comprise: administering an EGFR-TKI agent to the subject, and administering a microRNA mimic of miR-34, miR-126, miR-124, miR-147, and miR-215 to the subject Similar methods include contacting (e.g., treating) a cell or tissue (e.g., a cancer cell or cancer tissue such as a tumor) with an EGFR-TKI agent, and contacting the cell or tissue with a microRNA mimic of miR-34, miR-126, miR-124, miR-147, and miR-215. The synthetic oligonucleotide can comprise a sequence that is at least 80% (or 85, 90, 95, 100%) identical to at least one of SEQ ID NOs:1-6 and 168-179 (miR-34, miR-126, miR-124, miR-147, and miR-215, as well as family members, functional homologs, seed sequences, or consensus sequences thereof). These, and other, synthetic oligonucleotides can comprise natural nucleic acids, derivatives and chemically modified forms thereof, as well as nucleic acid analogs.

In various aspects and embodiments, the invention provides methods of administering an EGFR-TKI agent to a subject (e.g., a subject having cancer), and administering a microRNA mimic of a microRNAs listed in Appendix A as SEQ ID NOs:8-122 (downregulated microRNAs) to the subject. Similar methods include contacting a cell or tissue (e.g., a cancer cell or cancer tissue such as a tumor) with an EGFR-TKI agent, and contacting the cell or tissue with a microRNA mimic of a microRNAs listed in Appendix A as SEQ ID NOs:8-122 (downregulated microRNAs). The synthetic oligonucleotide can comprise a sequence that is at least 80% (or 85, 90, 95, 100%) identical to at least one of SEQ ID NOs:8-122.

In various aspects and embodiments, the invention provides methods of administering an EGFR-TKI agent to a subject (e.g., a subject having cancer), and administering an inhibitor of a microRNAs listed in Appendix A as SEQ ID NOs:123-167, preferably, SEQ ID NOs:156-167, more preferably, SEQ ID NOs:159, 164, and 165 (upregulated microRNAs). Similar methods include contacting a cell or tissue (e.g., a cancer cell or cancer tissue such as a tumor) with an EGFR-TKI agent, and contacting the cell or tissue with an inhibitor of a microRNAs listed in Appendix A as SEQ ID NOs:123-167, preferably, SEQ ID NOs:156-167, more preferably, SEQ ID NOs:159, 164, and 165 (upregulated microRNAs). The inhibitor can be a synthetic oligonucleotide comprising a sequence that is at least 80% (or 85, 90, 95, 100%) complementary to the microRNA.

In various embodiments, the EGFR-TKI agent can be erlotinib or an analogous EGFR-TKI agent such as gefitinib, afatinib, panitumumab, or cetuximab, or a HER2 inhibitor such as lapatinib, pertuzumab, or trastuzumab. In some embodiments, the EGFR inhibitor is erlotinib and the synthetic oligonucleotide is at least 80% (or 85, 90, 95, 100%) identical to one of SEQ ID NOs:1-4, for example SEQ ID NO:1.

In various embodiments, the cancer can be a cancer in which combinations of synthetic oligonucleotides and EGFR-TKI inhibitors in accordance with the present invention are effective therapeutics, for example lung cancer (e.g., non-small cell lung, NSCL) and liver cancer (e.g., hepatocellular carcinoma, HCC). The cancer can include a metastatic lesion in the liver.

In various embodiments, the cancer can be is resistant to treatment with the EGFR-TKI agent alone. The resistance can be primary or secondary (acquired). The cancer can be a lung (e.g., NSCL) cancer that has primary or secondary resistance to treatment with the EGFR-TKI agent alone. The cancer can be a liver cancer (e.g., HCC) that has primary or secondary resistance to treatment with the EGFR-TKI agent alone.

In various embodiments, the EGFR-TKI agent can be administered at an effective dose that is below (e.g., at least 50% below) the dose needed to be effective in the absence of the synthetic oligonucleotide administration. The dose can be 5, 10, 15, 20, 25, 30, 35, 40, 45, 50, 60, 70, 80, or 90% before the dose necessary in absence of the synthetic oligonucleotide.

In various embodiments, the IC₅₀ of the EGFR-TKI agent is reduced (e.g., at least 2-fold) relative to the IC₅₀ in the absence of the synthetic oligonucleotide administration. The IC₅₀ can be reduced by at least 1.5, 2, 2.5, 3, 4, 5, or 10 fold.

In various embodiments, the subject is a human, non-human primate, or laboratory animal (e.g., mouse, rat, guinea pig, rabbit, pig). The subject can have a KRAS mutation. The subject can have a EGFR mutation. In some embodiments, the subject has a primary or secondary resistance to erlotinib, for example, a patient who has developed or is likely to develop resistance to an EGFR-TKI agent. Alternatively, the subject's cancer may be sufficiently sensitive to the EGFR-TKI agent, however, that toxicity of the monotherapy may indicate that a lower dose of EGFR-TKI agent is desirable.

Various aspects, embodiments, and features of the invention are presented and described in further detail below. However, the foregoing and following descriptions are illustrative and explanatory only and are not restrictive of the invention, as claimed.

### BRIEF DESCRIPTION OF THE FIGURES

FIG. 1 illustrates generation of cell lines with secondary (acquired) resistance. HCC827 resistant cells were generated by treating the parental cells at low concentration of erlotinib (IC₁₀), and continually increasing the concentration up to IC₉₀ over 2-3 months.

FIGS. 2A-2C illustrate identification of novel miRNA candidates controlling erlotinib resistance. RNA was isolated from erlotinib-resistant HCC827 cells and tested on Agilent/Sanger12_0 miRNA arrays to identify miRNAs that are differentially expressed in HCC erlotinib-resistant cells versus the parental, erlotinib-sensitive cell line. miRNAs in thin and thick boxes are encoded on the same gene cluster, respectively. (FIG. 2A). FIG. 2B and FIG. 2C show data for genes and miRNAs, respectively, in bar graph format. CP, cisplatin; VC, vincristine; DA, daunorubicin; TZ, temozolodime; DR, doxorubicin; PT, paclitaxel; IFN, interferon; MDR, multidrug; A, apoptosis; C, cetuximab; G, gemcitabine; T, tamoxifen; M, methotrexate; 5-FU, 5-fluorouracil; AM, adriamycin.

FIGS. 3A-3C demonstrate the combinatorial effect of erlotinib and specific miRNAs. FIG. 3A: Determination of IC₅₀ values of erlotinib alone. FIG. 3B: Determination of IC₅₀ (or IC₂₀, or IC₂₅) values of miRNAs alone. FIG. 3C: Determination of combinatorial effects of miR-34a with erlotinib. miR-34 was reverse transfected at fixed, weak concentration (˜IC₂₅). Then, the cells were treated with erlotinib in a serial dilution. The combinatorial effect was evaluated by the visual inspection of the dose response curve and a shift of the IC₅₀ value.

FIGS. 4A-D illustrate an example of a microRNA mimic restoring EGFR-TKI sensitivity in cancer cells. FIG. 4A: Dose-dependent effect of erlotinib in parental HCC827 cells. Cells were treated with erlotinib in a serial dilution for 3 days, and cellular proliferation was determined by AlarmaBlue. FIG. 4B: HCC827 cells resistant to erlotinib (HCC827^{res}) were developed by incubating cells with increasing erlotinib concentrations over the course of 10 weeks until cells grew normally at concentrations equal to IC₉₀ in parental HCC827. FIGS. 4C and D: HCC827^{res} and H1299 cells were reverse-transfected with 0.3 nM miR-34a or miR-NC (negative control), and incubated in media supplemented with erlotinib in a serial dilution. After 3 days, cellular proliferation was determined. IC₅₀ values of erlotinib alone or in combination with miRNA are shown in the graphs.

FIGS. 5A-C illustrate an example of synergistic effects between a microRNA mimic and an EGFR-TKI agent in cancer cells, in particular between a miR-34a mimic and erlotinib in NSCLC cells. FIG. 5A: Combination index (CI) analysis. CI values were generated by linear regression and non-linear regression methods. Trendlines indicate CI values at any given effect (Fa, fraction affected, % inhibition), and symbols represent CI values derived from actual data points. CI=1, additivity; CI>1, antagonism; CI<1, synergy. FIG. 5B: Isobologram analysis. The diagonal, dotted line indicates additivity, and the square symbol shows dose requirements to achieve 50% and 80% (A549, H1299, H460) or 30% and 50% (H226) cancer cell inhibition, respectively. Data points below the line of additivity indicate synergy, data points above denote antagonism. FIG. 5C: Curve shift analysis. Data derived from non-linear regression trendlines were normalized to IC₅₀ values of the single agents (IC₅₀ eq) and plotted in the same graph. Left and right shifts of the dose-response curves of the combination (dotted line) relative to the dose-response curves of the single agents (grey, black) indicate synergy or antagonism, respectively. Actual experimental data points are shown.

FIGS. 6A-D illustrate an example of synergistic effects between a microRNA mimic and EGFR-TKI in cancer cells, in particular how certain ratios of erlotinib and miR-34a cooperate synergistically in A549 cells. FIG. 6A: Summary table showing potency (Fa), CI and DRI values of erlotinib and miR-34a combined at various concentrations and ratios. The molar miR-34-erlotinib ratios 1:533, 1:1333, 1:3333 (IC₅₀:IC₅₀ ratio), 1:8333, and 1:20833 are shown. FIG. 6B: Combination index plot of various drug ratios. CI values from actual data points are indicated by symbols. FIG. 6C: Isobologram at 80% cancer cell inhibition. Square symbols represent the 80% isobole of various ratios. The dotted line represents the isobole derived from actual erlotinib-miR-34a combinations that produced 80% (±2%) inhibition. FIG. 6D: Curve shift analysis of various drug ratios.

FIGS. 7A-C illustrate an example of synergistic effects between a microRNA mimic and EGFR-TKI in cancer cells, in particular how erlotinib and miR-34a synergize in HCC cells. FIG. 7A: Combination index analysis. FIG. 7B: Isobologram analysis. FIG. 7C: Curve shift analysis. See FIG. 5 for explanation of graphs.

FIGS. 8A-C illustrates endogenous miR-34 and mRNA levels of genes controlling erlotinib resistance in NSCLC cells. The data is divided into three parts: FIG. 8A (miR-34a, miR-34b, FGFR1, and KRAS), FIG. 8B (miR-34c, EGFR, ERBB3, and PIK3CA), and FIG. 8C (AXL, GAS6, MET, and HGF). Total RNA was used in triplicate qRT-PCR to measure miR-34a/b/c and mRNA levels of genes implicated in erlotinib resistance. Data were normalized to house-keeping miRNAs and mRNAs, respectively, and expressed as percent change compared to levels in HCC827 cells. u, undetected.

FIGS. 9A-B illustrates dose-response curves of the single agents in NSCLC cells resistant to erlotinib. The data is divided into two parts: FIG. 9A (A549 and H1299) and FIG. 9B (H460 and H226). Cells were treated in triplicates with erlotinib or miR-34a alone at indicated concentrations. Cellular proliferation was measured 3 days or 4 days after erlotinib treatment or miR-34a reverse transfection, respectively. Non-linear regression trendlines were generated using Graphpad, and IC₅₀ and IC₂₅ values were calculated. Goodness of fit of non-linear regression trendlines is indicated by R² values. The asterisk denotes theoretical IC₅₀ values derived from an extrapolation of the dose-response curve (H226).

FIGS. 10A-D illustrates summary tables showing potency, CI and DRI values of erlotinib and miR-34a combined at various concentrations and ratios in NSCLC cells. Combinations that yield Fa>65%, CI<0.6, DRI>2 are highlighted in grey and are considered relevant. The data is divided into four parts: FIG. 10A (A549), FIG. 10B (H1299), FIG. 10C (H460), and FIG. 10D (H226). Fa, fraction affected (% inhibition of cellular proliferation); CI, combination index; DRI, dose reduction index.

FIG. 11 illustrates endogenous expression of miR-34 and mRNAs of genes controlling erlotinib resistance in HCC cells. Total RNA was used in triplicate qRT-PCR to measure miR-34a/b/c and mRNA levels of genes implicated in erlotinib resistance. Data were normalized to house-keeping miRNAs and mRNAs, respectively, and expressed as percent change compared to levels in HCC827 cells. u, undetected.

FIGS. 12A-B illustrates dose-response curves of the single agents in HCC cells resistant to erlotinib. The data is divided into two parts: FIG. 12A (Hep3B and C3A) and FIG. 12B (HepG2 and Huh7). Cells were treated in triplicates with erlotinib or miR-34a alone at indicated concentrations. Cellular proliferation was measured 3 days or 6 days after erlotinib treatment or miR-34a reverse transfection, respectively. Non-linear regression trendlines were generated using Graphpad, and IC₅₀ and IC₂₅ values were calculated. Goodness of fit of non-linear regression trendlines is indicated by R² values. The asterisk denotes theoretical IC₅₀ values of erlotinib derived from an extrapolation of the dose-response curve (Hep3B, C3A, HepG2).

FIGS. 13A-D illustrates summary tables showing potency, CI and DRI values of erlotinib and miR-34a combined at various concentrations and ratios in HCC cells. The data is divided into four parts: FIG. 13A (Hep3B), FIG. 13B (C3A), FIG. 13C (HepG2), and FIG. 13D (Huh7). Combinations that yield Fa>65%, CI<0.6, DRI>2 are highlighted in grey and are considered relevant. Fa, fraction affected (% inhibition of cellular proliferation); CI, combination index; DRI, dose reduction index.

FIG. 14 illustrates data showing that miR-34-Mim synergized with lapatinib across four tested breast cancer cell lines (BT-549, MCF-7, MDA-MB-231, T47D). Symbols represent CI values derived from actual data points. CI, combination index; Fa, fraction affected (=inhibition of proliferation); CI=1, additivity; CI>1, antagonism; CI<1, synergy.

### DETAILED DESCRIPTION OF THE INVENTION

The invention is based, in part, on the discovery that certain microRNAs can be consistently up- or down-regulated in EGFR-TKI-resistant cell lines, and that specific combinations of synthetic oligonucleotides and EGFR-TKI agents can have advantageous and/or unexpected results, for example because they are particularly efficacious in treating certain cells (e.g., synergize, or have greater that additive effect). Accordingly, the invention, in various aspects and embodiments includes contacting cells, tissue, and/or organisms with specific combinations of synthetic oligonucleotides and EGFR-TKI agents. More particularly, the invention can include contacting cancer cells, cancer tissue, and/or organisms having cancer with such combinations of synthetic oligonucleotides and EGFR-TKI agents. The methods can be experimental, diagnostic, and/or therapeutic. The methods can be used to inhibit, or reduce the proliferation of, cells, including cells in a tissue or an organism. The synthetic oligonucleotides can be, for example, mimics or inhibitors of microRNAs that are consistently down- or up-regulated in EGFR-TKI-resistant cells lines.

### Synthetic Oligonucleotides

microRNAs (miRNAs) are small non-coding, naturally occurring RNA molecules that post-transcriptionally modulate gene expression and determine cell fate by regulating multiple gene products and cellular pathways (Bartel, Cell, 2004. 116(2):281-97). miRNAs interfere with gene expression by either degrading the mRNA transcript by blocking the protein translation machinery (Bartel, supra). miRNAs target mRNAs with sequences that are fully or merely partially complementary which endows these regulatory RNAs with the ability to target a broad but nevertheless specific set of mRNAs. To date, there are about 2,500 human annotated mature miRNA sequences with roles in processes as diverse as cell proliferation, differentiation, apoptosis, stem cell development, and immune function (Costinean et al., Proc Natl Acad Sci USA, 2006. 103(18):7024-9). Often, the misregulation of miRNAs can contribute to the development of human disease including cancer (Esquela-Kerscher et al., Nat Rev Cancer, 2006. 6(4):259-69; Calin et al., 2006. 6(11):857-66). miRNAs deregulated in cancer can function as bona fide tumor suppressors or oncogenes. A single miRNA can target multiple oncogenes and oncogenic signaling pathways (Forgacs et al., Pathol Oncol Res, 2001. 7(1):6-13), and translating this ability into a future therapeutic may hold the promise of creating a remedy that is effective against tumor heterogeneity. Thus, miRNAs have the potential of becoming powerful therapeutic agents for cancer (Volinia et al., Proc Natl Acad Sci USA, 2006. 103(7):2257-61; Tong et al., Cancer Gene Ther, 2008. 15(6):341-55) that act in accordance with our current understanding of cancer as a “pathway disease” that can only be successfully treated when intervening with multiple cancer pathways (Wiggins et al., Cancer Res, 2010. 70(14): 5923-5930.; Jones et al., Science, 2008. 321(5897):1801-6; Parsons et al., Science, 2008. 321(5897):1807-12).

As of March 2013, Mirna Therapeutics (Austin, Tex.) has completed the preclinical development program to support the manufacture of cGMP-materials and the conduction of IND-enabling studies for a miR-34-based supplementation therapy (MRX34). Mirna evaluated the toxicity as well as the pharmacokinetic profile of the formulation containing miR-34 mimic in non-GLP pilot studies using mice, rats and non-human primates. These experiments did not show adverse events at the predicted therapeutic levels of MRX34, as measured by clinical observations, body weights, clinical chemistries (including LFT, RFT and others), hematology, gross pathology, histopathology of select organs and complement (CH₅₀). In addition, miRNA mimics formulated in lipid nanoparticles do not induce the innate immune system as demonstrated in fully immunocompetent mice, rats, non-human primates, as well as human whole blood specimens. A more detailed review of the pre-clinical data is provided in Bader, Front Genet. 2012; 3:120.

In methods of the inventions, a specific synthetic oligonucleotide is administered to a subject as part of a combination therapy with an EGFR-TKI agent. A synthetic oligonucleotide can be a microRNA mimic or a microRNA inhibitor. A synthetic oligonucleotide can have a conventional naturally occurring sequences (provided herein), as well as any chemically modified versions and sequence homologues thereof. Administering a synthetic oligonucleotide can include administering a microRNA vector, such as a viral vector, for example, to synthetically induce expression of a microRNA. Administering a synthetic oligonucleotide can include administering a synthetic microRNA precursor, or synthetically inducing the expression of a microRNA precursor. Administering a synthetic oligonucleotide can include administering a synthetic microRNA in hairpin form, for example a hairpin loop structure.

In various aspects and embodiments, the present invention employs a microRNA mimic or inhibitor, which is not delivered through transfection into a cell. Rather, in various embodiments, the synthetic oligonucleotide can be administered by methods such as injection or transfusion. In some embodiments, rather than an isolated cell, tissue, or culture thereof, the subject can be a mammal (e.g., a human or laboratory animal such as a mouse, rat, guinea pig, rabbit, pig, non-human primate, and the like).

The synthetic oligonucleotides used in connection with the invention can be 7-130 nucleotides long, double stranded RNA molecules, either having two separate strands or a hairpin structure. For example, a synthetic oligonucleotide can be 7, 8, 9, 10, 12, 13, 14, 15, 16, 17, 18, 19, 20, 21, 22, 23, 24, 25, 26, 27, 28, 29, 30, 7-30, 7-25, 15-30, 15-25, 17-30, or 17-25 nucleotides long. One of the two strands, which is referred to as the “guide strand”, contains a sequence which is identical or substantially identical to the seed sequence (nucleotide positions 2-9) of the parent microRNA sequence shown in the table below. “Substantially identical”, as used herein, means that at most 1 or 2 substitutions and/or deletions are allowed. In some embodiments, the guide strand comprises a sequence which is at least 80%, 85%, 90%, 95% identical to the respective full length sequence provided herein. The second of the two strands, which is referred to as a “passenger strand”, contains a sequence that is complementary or substantially complementary to the seed sequence of the corresponding given microRNA. “Substantially complementary”, as used herein, means that at most 1 or 2 mismatches and/or deletions are allowed. In some embodiments, the passenger strand comprises a sequence which is at least 60%, 65%, 70%, 75%, 80%, 85%, 90%, 95% identical to the complement of the respective full length sequence provided herein. In some embodiments, the synthetic oligonucleotide is a mimic of miR-34a, miR-34b, miR-34c, miR-449a, miR-449b, miR-449c, miR-192, miR-215, miR-126, miR-124, miR-147, or an analog or homolog thereof. In some embodiments, the synthetic oligonucleotide includes the seed sequence of one of these microRNAs.

TABLE 1
microRNA Sequences and
Sequence Identification Numbers

| microRNA | Sequence | SEQ ID NO: |
|---|---|---|
| miR-34a | UGGCAGUGUCUUAGCUGGUUGUU | SEQ ID NO: 1 |
| miR-34b | UAGGCAGUGUCAUUAGCUGAUUG | SEQ ID NO: 168 |
| miR-34c | AGGCAGUGUAGUUAGCUGAUUGC | SEQ ID NO: 169 |
| miR-34 | *GGCAGUGU*UUAGCUG*UUG* | SEQ ID NO: 2 |
| consensus | | |
| miR-449a | UGGCAGUGUAUUGUUAGCUGGU | SEQ ID NO: 170 |
| miR-449b | AGGCAGUGUAUUGUUAGCUGGC | SEQ ID NO: 171 |
| miR-449c | UAGGCAGUGUAUUGCUAGCGGCUGU | SEQ ID NO: 172 |
| miR-449 | UGGCAGUGUAUUG*UAGC*G*G | SEQ ID NO: 173 |
| consensus | | |
| miR-34/ | GGCAGUG | SEQ ID NO: 174 |
| 449 seed | | |
| miR-101 | UACAGUACUGUGAUAACUGAA | SEQ ID NO: 7 |
| miR-124 | UUAAGGCACGCGGUGAAUGCCA | SEQ ID NO: 4 |
| miR-124 | UAAGGCA | SEQ ID NO: 175 |
| seed | | |
| miR-126 | UCGUACCGUGAGUAAUAAUGC | SEQ ID NO: 3 |
| miR-126 | CGUACCG | SEQ ID NO: 176 |
| seed | | |
| miR-147 | GUGUGUGGAAAUGCUUCUGC | SEQ ID NO: 5 |
| miR-147 | UGUGUGG | SEQ ID NO: 177 |
| seed | | |
| miR-192 | CUGACCUAUGAAUUGACAGCC | SEQ ID NO: 178 |
| miR-215 | AUGACCUAUGAAUUGACAGAC | SEQ ID NO: 6 |
| miR-192/ | UGACCUA | SEQ ID NO: 179 |
| 215 seed | | |

*denotes a deletion or any nucleotide(s).
Seed sequences are shown in bold highlighting.

The synthetic oligonucleotides (e.g., microRNA mimics) can be formulated in liposomes such as, for example, those described in U.S. Pat. Nos. 7,858,117 and 7,371,404; US Patent Application Publication Nos. 2009-0306194 and 2011-0009641. Other delivery technologies are known in the art and available, including expression vectors, lipid or various ligand conjugates.

In certain embodiments, methods of the invention include administering an inhibitor of a microRNA selected from the microRNAs listed in Appendix A as SEQ ID NOs:123-167, preferably, SEQ ID NOs:156-167, more preferably, SEQ ID NOs:159, 164, and 165. Inhibitors of microRNA are well known in the art and are typically antisense molecules that are complementary to the target microRNA, however, other types of inhibitors can also be used. Inhibitors of microRNAs are described, for example, in U.S. Pat. No. 8,110,558. In certain embodiments, an inhibitor of a microRNA contains a 9-20, 10-18, or 12-17 nucleotide long sequence that is complementary or substantially complementary to the corresponding upregulated microRNA sequence listed in Appendix A as SEQ ID NOs:123-167, preferably, SEQ ID NOs:156-167, more preferably, SEQ ID NOs:159, 164, and 165.

microRNAs and their inhibitors can also be chemically modified, for example, synthetic oligonucleotides may have a 5′ cap on the passenger strand (e.g., NH₂—(CH₂)₆—O—) and/or a mismatch at the first and/second nucleotide of the same strand. Other possible chemical modifications can include backbone modifications (e.g., phosphorothioate, morpholinos), ribose modifications (e.g., 2′-OMe, 2′-Me, 2′-F, 2′-4′-locked/bridged sugars (e.g., LNA, ENA, UNA) as well as nucleobase modifications (see, e.g., Peacock et al, 2011. J Am Chem Soc., 133(24):9200-9203. In certain embodiments, the synthetic oligonucleotides, and in particular, miR-34 and miR-124 mimics have modifications as described in U.S. Pat. No. 7,960,359 and US Patent Application Publication Nos. 2012-0276627 and 2012-0288933.

In some embodiments, the synthetic oligonucleotide is between 17 and 30 nucleotides in length and comprises (i) a microRNA region having a sequence from 5′ to 3′ that is at least 80% identical to at least one of SEQ ID NO:1-6, 8-122, or 168-179, and (ii) a complementary region having a sequence from 5′ to 3′ that is 60-100% complementary to the microRNA region.

In some embodiments, the synthetic oligonucleotide comprises a sequence that is at least 80, 85, 90, 95, or 100% identical to at least one of SEQ ID NO:1-6, 8-122, or 168-179.

In some embodiments, the synthetic oligonucleotide comprises a single polynucleotide or a double stranded polynucleotide. In some embodiments, the synthetic oligonucleotide comprises a hairpin polynucleotide.

In some embodiments, the synthetic oligonucleotide is between 17 and 30 nucleotides in length and comprises (i) a first polynucleotide having a sequence with at least 80% identical to at least one of SEQ ID NO:1-6, 8-122, or 168-179; and (ii) a separate second polynucleotide having a sequence from 5′ to 3′ that is 60-100% complementary to the first polynucleotide.

In some embodiments, the synthetic oligonucleotide is between 17 and 30 nucleotides in length and comprises one or more of the following (i) a replacement group for phosphate or hydroxyl of the nucleotide at the 5′ terminus of the complementary strand of the RNA molecule; (ii) one or more sugar modifications in the first or last 1 to 6 residues of the complementary region; or (iii) noncomplementarity between one or more nucleotides in the last 1 to 5 residues at the 3′ end of the complementary region and the corresponding nucleotides of the microRNA region.

In some embodiments, the synthetic oligonucleotide is between 17 and 30 nucleotides in length and comprises (i) at least one modified nucleotide that blocks the 5′ OH or phosphate at the 5′ terminus, wherein the at least one nucleotide modification is an NH2, biotin, an amine group, a lower alkylamine group, an acetyl group or 2′oxygen-methyl (2′O-Me) modification; or (ii) at least one ribose modification selected from 2′F, 2′NH₂, 2′N₃, 4′thio, or 2′O—CH₃.

In some embodiments, the synthetic oligonucleotide is between 17 and 30 nucleotides in length and comprises (i) a first polynucleotide having a sequence with at least 80% identical to at least one of SEQ ID NO:1-6, 8-122, or 168-179; (ii) a separate second polynucleotide having a sequence from 5′ to 3′ that is 60-100% complementary to the first polynucleotide; and (iii) a lower alkylamine group at the 5′ end of the complementary strand.

In some embodiments, the synthetic oligonucleotide is between 17 and 30 nucleotides in length and comprises (i) a first polynucleotide having 100% identical to at least one of SEQ ID NO:1-6, 8-122, or 168-179; (ii) a separate second polynucleotide having a sequence from 5′ to 3′ that is 100% complementary to the first polynucleotide; and (iii) a lower alkylamine group at the 5′ end of the complementary strand.

Synthetic oligonucleotides can be administered intravenously as a slow-bolus injection at doses ranging 0.001-10.0 mg/kg per dose, for example, 0.01-3.0, 0.025-1.0 or 0.25-0.5 mg/kg per dose, with one, two, three or more doses per week for 2, 4, 6, 8 weeks or longer as necessary.

### EGFR-TKI Agents

Methods of the invention involve administering an EGFR-TKI agent to a subject. The family of epidermal growth factor receptors (EGFR) comprises four structurally related cell-surface receptor tyrosine kinases that bind and elicit functions in response to members of the epidermal growth factor (EGF) family. In humans, this includes EGFR, also known as Her-1 and ErbB1, Her-2, also referred to as Neu and ErbB2, Her-3 (ErbB3), and Her-4 (ErbB4). Hyperactivation of ErbB signaling is associated with the development of a wide variety of solid tumors. Accordingly, in various additional embodiments, the present invention includes combinations of synthetic oligonucleotides with erlotinib as well as other EGFR inhibitors, such as gefitinib, afatinib, panitumumab and cetuximab, as well as HER2 inhibitors such as lapatinib, pertuzumab and trastuzumab.

In certain embodiments, the EGFR-TKI is erlotinib, the active ingredient of the drug currently marketed under the trade name TARCEVA®. Unless expressly stated otherwise, the term “erlotinib” herein refers the compound of Formula I, as well as to any of its salts or esters thereof.

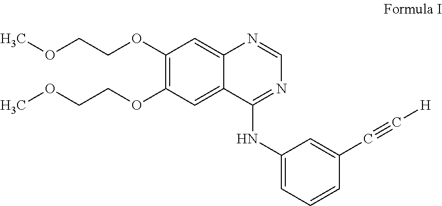 [SMILES: C#Cc1cccc(Nc2ncnc3cc(OCCOC)c(OCCOC)cc23)c1]

Erlotinib is a tyrosine kinase inhibitor, a quinazolinamine with the chemical name N-(3-ethynylphenyl)-6,7-bis(2-methoxyethoxy)-4-quinazolinamine. In specific embodiments, the erlotinib is erlotinib hydrochloride. TARCEVA® tablets for oral administration are available in three dosage strengths containing erlotinib hydrochloride (27.3 mg, 109.3 mg and 163.9 mg) equivalent to 25 mg, 100 mg and 150 mg erlotinib and the following inactive ingredients: lactose monohydrate, hypromellose, hydroxypropyl cellulose, magnesium stearate, microcrystalline cellulose, sodium starch glycolate, sodium lauryl sulfate and titanium dioxide. The tablets also contain trace amounts of color additives, including FD&C Yellow #6 (25 mg only) for product identification. Further information is available from the approved drug label.

Erlotinib is also described in U.S. Pat. No. 6,900,221, herein incorporated by reference, and the corresponding PCT Publication WO 01/34574.

The approved recommended dose of TARCEVA® for NSCLC is 150 mg/day; the approved dose for pancreatic cancer is 100 mg/day. Doses may be reduced in 50 mg decrements when necessary.

In certain embodiments where the EGFR-TKI agent is erlotinib, the synthetic oligonucleotide does not have the sequence of miR-126 (e.g., less than 100, 95, 90, 85, or 80% identity with the sequence of human miR-126 or seed sequence thereof).

In other embodiments, the EGFR-TKI agent is gefitinib, the active ingredient of the drug marketed under the trade name IRESSA®. Unless expressly stated otherwise, the term “gefitinib” refers herein the compound of Formula II, as well as to any of salts or esters thereof.

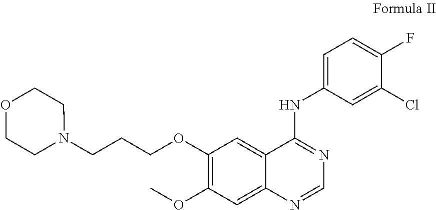 [SMILES: COc1cc2ncnc(Nc3ccc(F)c(Cl)c3)c2cc1OCCCN1CCOCC1]

Gefitinib is a tyrosine kinase inhibitor with the chemical name 4-quinazolinamine, N-(3-chloro-4fluorophenyl)-7-methoxy-6-[3-4-morpholin) propoxy], and also is known as ZD1839. The clinical formulation is supplied as 250 mg tablets, containing the active ingredient, lactose monohydrate, microcrystalline cellulose, croscarmellose sodium, povidone, sodium lauryl sulfate and magnesium stearate. The recommended dose as a single therapy is one 250 mg tablet per day. Further information can be found on the approved drug label.

Other EGFR inhibitors, such as afatinib, panitumumab and cetuximab, as well as HER2 inhibitors such as lapatinib, pertuzumab and trastuzumab are known in the art and, thus, a person of ordinary skill would readily know their structure, formulation, dosing, and administration, etc. (e.g., based on published medical information such as an approved drug label) as would be required in use with the present invention.

### Cancer

The invention provides methods and compositions for treating cancer cells and/or tissue, including cancer cells and/or tissue in a subject, or in vitro treatment of isolated cancer cells and/or tissue. If in a subject, the subject to be treated can be an animal, e.g., a human or laboratory animal.

The subject being treated may have been diagnosed with cancer, for example, lung cancer (non-small cell lung cancer (NSCLC), e.g., adenocarcinoma, squamous cell carcinoma, and large cell carcinoma), pancreatic cancer, or cancer in the liver, or any other type of cancer that benefits from a EGRF inhibition, including breast cancer, HCC, colorectal cancer, head and neck cancers, prostate, brain, stomach, or bladder cancer. In some embodiments, the cells or the subject have/has a primary or secondary resistance to an EGFR-TKI agent.

The subject may have locally advanced, unresectable, or metastatic cancer and/or may have failed a prior first-line therapy. In some embodiments, the subject has undergone a prior treatment with an EGRR-TKI agent lasting at least 2, 4, 6, 8, 10 months or longer. In other embodiments, the subject has the T790M mutation in EGFR (Balak et al. 2006. Clin Cancer Res, 12(1):6494-501). In other embodiments, the subjects are patients who have experienced one or more significant adverse side effect to an EGFR-TKI agent and therefore require a reduction in dose. The subject being treated may also be the one characterized by one of the following: (1) K-RAS mutation; (2) amplification and overexpression of c-Met; (3) BRAF mutation; (4) ALK translocation (5) hepatocyte growth factor (HGF) overexpression; (6) other EGFR mutations (small insertions or duplications in exon 20: D770 N771, ins NPG, ins SVQ, ins G and N771T; and (7) genetic lesions that affect signaling downstream of EGFR, including PIK3CA, loss of PTEN, IGF1R and KDM5A.

In various embodiments, the cancer is liver cancer (e.g., HCC). The liver cancer may not be resistant to an EGFR-TKI agent. Alternatively, the liver cancer (e.g., HCC) can have primary or secondary resistance to an EGFR-TKI agent. The subject can be a responder to an EGFR-TKI agent in the absence of the synthetic oligonucleotide. The subject can be a non-responder to a EGFR-TKI in the absence of the synthetic oligonucleotide. In some embodiments, the subject has undergone a prior treatment with the EGFR-TKI agent lasting at least 2, 4, 6, 8, 10 months or longer. In other embodiments, the subjects are patients who have experienced one or more significant adverse side effect to the EGFR-TKI agent and therefore require a reduction in dose.

In various embodiments, the liver cancer (e.g., HCC) can be intermediate, advanced, or terminal stage. The liver cancer (e.g., HCC) can be metastatic or non-metastatic. The liver cancer (e.g., HCC) can be resectable or unresectable. The liver cancer (e.g., HCC) can comprise a single tumor, multiple tumors, or a poorly defined tumor with an infiltrative growth pattern (into portal veins or hepatic veins). The liver cancer (e.g., HCC) can comprise a fibrolamellar, pseudoglandular (adenoid), pleomorphic (giant cell), or clear cell pattern. The liver cancer (e.g., HCC) can comprise a well differentiated form, and tumor cells resemble hepatocytes, form trabeculae, cords, and nests, and/or contain bile pigment in cytoplasm. The liver cancer (e.g., HCC) can comprise a poorly differentiated form, and malignant epithelial cells are discohesive, pleomorphic, anaplastic, and/or giant. In some embodiments, the liver cancer (e.g., HCC) is associated with hepatits B, hepatitis C, cirhhosis, or type 2 diabetes.

In some embodiments, the therapeutically effective dose of an EGFR-TKI agent is reduced. For example, the weekly or monthly dose of the EGFR-TKI agent reduced by at least 10%, 20%, 30%, 40%, 50%, 60%, 70%, 80%, 90% or more relative to the maximum recommended dose or the maximum tolerated dose. In other embodiments, the EGFR-TKI agent is administered at an effective dose that at least 50%, 60%, 70%, 80%, 90% or more below the dose needed to be effective in the absence of the synthetic oligonucleotide microRNA mimic (or microRNA inhibitor) administration. For example, erlotinib can be administered at a dose of 50, 40, 30, 25 mg per day or less. In some embodiments, the IC₅₀ of an EGFR-TKI agent is reduced by at least 4-, 5-, 10-, 20-, 30-, 40-, 50-, or 100-fold relative to the IC₅₀ in the absence of the synthetic oligonucleotide microRNA mimic (or microRNA inhibitor) treatment. IC₅₀ can be determined, for example, as illustrated in the Examples.

### Combination Chemotherapy

Combination chemotherapy or polytherapy is the use of more than one medication or other therapy (e.g., as opposed to monotherapy, which is any therapy taken alone). As used herein with reference to the present invention, the term refers to using specific combinations of EGFR-TKI agents and synthetic oligonucleotides.

As used herein for describing ranges, e.g., of ratios, doses, times, and the like, the terms “about” embraces variations that are within the relevant margin of error, essentially the same (e.g., within an art-accepted confidence interval such as 95% for phenomena that follow a normal or Gaussian distribution), or otherwise does not materially change the effect of the thing being quantified.

The EGFR-TKI agent dosing amount and/or schedule can follow clinically approved, or experimental, guidelines. Further to the description in the EGFR-TKI agents section, in various embodiments, the dose of EGFR-TKI agent can be a dose prescribed by the FDA drug label, or label/instructions of another agency.

Likewise the synthetic oligonucleotide dosing amount and/or schedule can follow clinically approved, or experimental, guidelines. In various embodiments, the dose of synthetic oligonucleotide is about 10, 20, 25, 30, 40, 50, 75, 100, 125, 150, 175, 200, 225, or 250 mg/m² per day.

In various embodiments the synthetic oligonucleotide is administered to the subject in 1, 2, 3, 4, 5, 6, or 7 daily doses over a single week (7 days). The synthetic oligonucleotide can be administered to the subject in 1, 2, 3, 4, 5, 6, 7, 8, 9, 10, 11, 12, 13, or 14 daily doses over 14 days. The synthetic oligonucleotide can be administered to the subject in 1, 2, 3, 4, 5, 6, 7, 8, 9, 10, 11, 12, 13, 14, 15, 16, 17, 18, 19, 20, or 21 daily doses over 21 days. The synthetic oligonucleotide can be administered to the subject in 1, 2, 3, 4, 5, 6, 7, 8, 9, 10, 11, 12, 13, 14, 15, 16, 17, 18, 19, 20, 21, 22, 23, 24, 25, 26, 27, or 28 daily doses over 28 days.

In various embodiments the synthetic oligonucleotide is administered for: 2 weeks (total 14 days); 1 week with 1 week off (total 14 days); 3 consecutive weeks (total 21 days); 2 weeks with 1 week off (total 21 days); 1 week with 2 weeks off (total 21 days); 4 consecutive weeks (total 28 days); 3 consecutive weeks with 1 week off (total 28 days); 2 weeks with 2 weeks off (total 28 days); 1 week with 3 consecutive weeks off (total 28 days).

In various embodiments the synthetic oligonucleotide is: administered on day 1 of a 7, 14, 21 or 28 day cycle; administered on days 1 and 15 of a 21 or 28 day cycle; administered on days 1, 8, and 15 of a 21 or 28 day cycle; or administered on days 1, 2, 8, and 15 of a 21 or 28 day cycle. The synthetic oligonucleotide can be administered once every 1, 2, 3, 4, 5, 6, 7, or 8 weeks.

A course of EGFR-TKI agent-synthetic oligonucleotide therapy can be prescribed by a clinician. The combination therapy can be administered for 1, 2, 3, 4, 5, 6, 7, 8, 9, 10, 11, or 12 cycles.

A course of EGFR-TKI agent-synthetic oligonucleotide therapy can be continued until a clinical endpoint is met. In some embodiments, the therapy is continued until disease progression or unacceptable toxicity occurs. In some embodiments, the therapy is continued until achieving a pathological complete response (pCR) rate defined as the absence of cancer. In some embodiments, the therapy is continued until partial or complete remission of the cancer. Administering the synthetic oligonucleotide and the EGFR-TKI agent to a plurality of subject having cancer may increase the Overall Survival (OS), the Progression free Survival (PFS), the Disease Free Survival (DFS), the Response Rate (RR), the Quality of Life (QoL), or a combination thereof.

In various embodiments, the treatment reduces the size and/or number of the cancer tumor(s); prevent the cancer tumor(s) from increasing in size and/or number; and/or prevent the cancer tumor(s) from metastasizing.

In the methods of the invention, administration is not necessarily limited to any particular delivery system and may include, without limitation, parenteral (including subcutaneous, intravenous, intramedullary, intraarticular, intramuscular, or intraperitoneal injection), rectal, topical, transdermal, or oral (for example, in capsules, suspensions, or tablets). Administration to an individual may occur in a single dose or in repeat administrations, and in any of a variety of physiologically acceptable salt forms, and/or with an acceptable pharmaceutical carrier and/or additive as part of a pharmaceutical composition. Physiologically acceptable salt forms and standard pharmaceutical formulation techniques, dosages, and excipients are well known to persons skilled in the art (see, e.g., Physicians' Desk Reference (PDR®) 2005, 59^{th} ed., Medical Economics Company, 2004; and Remington: The Science and Practice of Pharmacy, eds. Gennado et al. 21th ed., Lippincott, Williams & Wilkins, 2005).

Additionally, effective dosages achieved in one animal may be extrapolated for use in another animal, including humans, using conversion factors known in the art. See, e.g., Freireich et al., Cancer Chemother Reports 50(4):219-244 (1966) and Table 2 for equivalent surface area dosage factors). Reports 50(4):219-244 (1966) and Table 2 for equivalent surface area dosage factors).

TABLE 2
equivalent surface area dosage factors

From:

| | Mouse | Rat | Monkey | Dog | Human |
|---|---|---|---|---|---|
| To: | (20 g) | (150 g) | (3.5 kg) | (8 kg) | (60 kg) |
| Mouse | 1 | 0.5 | 0.25 | 0.17 | 0.08 |
| Rat | 2 | 1 | 0.5 | 0.25 | 0.14 |
| Monkey | 4 | 2 | 1 | 0.6 | 0.33 |
| Dog | 6 | 4 | 1.7 | 1 | 0.5 |
| Human | 12 | 7 | 3 | 2 | 1 |

In various embodiments, the synthetic oligonucleotide is administered prior to the EGFR-TKI agent, concurrently with the EGFR-TKI agent, after the EGFR-TKI agent, or a combination thereof. The synthetic oligonucleotide can be administered intravenously. The synthetic oligonucleotide can be administered systemically or regionally.

The combination therapies of the invention are not specifically limited to any particular course or regimen and may be employed separately or in conjunction with other therapeutic modalities (e.g., chemotherapy or radiotherapy).

A combination therapy in accordance with the present invention can include additional therapies (e.g., pharmaceutical, radiation, and the like) beyond the EGFR-TKI agent and synthetic oligonucleotide Similarly, the present invention can be used as an adjuvant therapy (e.g., when combined with surgery). In various embodiments, the subject is also treated by surgical resection, percutaneous ethanol or acetic acid injection, transcatheter arterial chemoembolization, radiofrequency ablation, laser ablation, cryoablation, focused external beam radiation stereotactic radiotherapy, selective internal radiation therapy, intra-arterial iodine-131-lipiodol administration, and/or high intensity focused ultrasound.

The combination of the synthetic oligonucleotide and EGFR-TKI agent can be used as an adjuvant, neoadjuvant, concomitant, concurrent, or palliative therapy. The combination of the synthetic oligonucleotide and EGFR-TKI agent can be used as a first line therapy, second line therapy, or crossover therapy.

In some embodiments, the therapeutically effective dose of EGFR-TKI agent is reduced through combination with the synthetic oligonucleotide. For example, the daily, weekly, or monthly dose of EGFR-TKI agent can be reduced by at least 10%, 20%, 30%, 40%, 50%, 60%, 70%, 80%, 90% or more relative to the maximum recommended dose or the maximum tolerated dose. In other embodiments, EGFR-TKI agent is administered at an effective dose that at least 50%, 60%, 70%, 80%, 90% or more below the dose needed to be effective in the absence of the synthetic oligonucleotide microRNA mimic (or microRNA inhibitor) administration. In some embodiments, the IC₅₀ of EGFR-TKI agent is reduced by at least 4-, 5-, 10-, 20-, 30-, 40-, 50-, or 100-fold relative to the IC₅₀ in the absence of the synthetic oligonucleotide.

Further description and embodiments of combination therapies are provided in the Examples section below.

As discussed and further illustrated in the examples below, the present invention provides methods and compositions for treating cancer (e.g., lung or liver cancer) where the EGFR-TKI agent and synthetic oligonucleotide are administered in a combination that is particularly effective (e.g., synergistic or more than additive). While synergy and synonymous terms are commonly used in the art, the property is not always defined or quantified (and, hence, the purported synergy may not actually be present). In connection with the present invention and the examples below, combination index (CI) values were used to quantify the effects of various combinations of EGFR-TKI agent and synthetic oligonucleotide.

In various embodiments, the combination of EGFR-TKI agent and synthetic oligonucleotide exhibits a CI<1 in the cancer (e.g., lung cancer or liver cancer). The combination can exhibits a CI<0.95, 0.90, 0.85, 0.80, 0.75, 0.70, 0.65, 0.60, 0.55, 0.50, 0.45, 0.40, 0.35, 0.30, 0.25, or 0.20 in the cancer).

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

### Selection of Erlotinib-Resistant Cell Lines

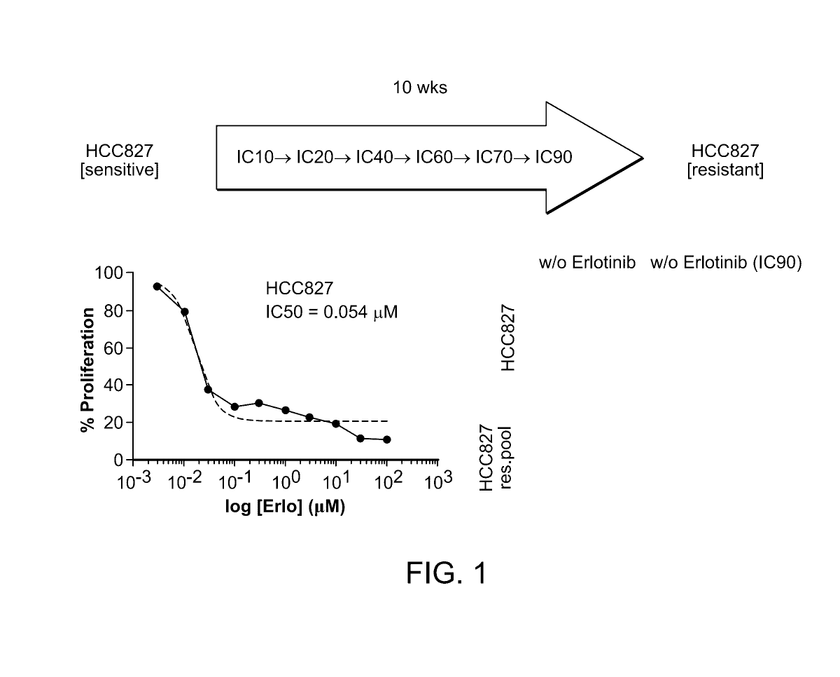

We followed a protocol described in Engelman et al. (supra) to generate NSCLC lines with acquired resistance to erlotinib. Briefly, parental HCC827 cells highly sensitive to erlotinib (IC₅₀ₑᵣₗₒ=0.054 μM) were incubated with erlotinib at increasing concentrations over 10 weeks until cells were able to proliferate in medium containing erlotinib at a concentration that is equivalent to IC₉₀ in parental HCC827 cells. Over the course of the selection, 3 cell lines from individual cell clones were obtained (HCC827^{clone 5,6,7}). In addition, we obtained a heterogenic mass culture presumably originating from multiple clones (HCC827^{res.pool}) (see FIG. 1).

Table 3 provides the list of 4 NSCLC cells used to assess the combinatorial effects of miRNAs and EGFR-TKIs. The particular cell lines were selected based on the IC₅₀ values of EGFR-TKIs in these cells, their oncogenic properties and their susceptibility to miRNAs. This list includes cell lines that are resistant to erlotinib, and cells that are sensitive. The IC₅₀ values of erlotinib for each of these cell lines as reported in the scientific literature are shown. In these examples, cell lines with IC₅₀ values >1 μM are considered resistant.

TABLE 3

| Cell line | Histology | Gene mutation | IC₅₀ [Erl] | |
|---|---|---|---|---|
| H1299 | AC | NRAS, TP53 | 8.6-38 | μM |
| (resistant) | | | | |
| H460 | LCC | KRAS, STK11, | 8-24 | μM |
| (resistant) | | CDKN2A, PIK3CA | | |
| HCC827^{res. pool} | AC | EGFR | N/R* | |
| (resistant) | | | | |
| HCC827 parental | AC | EGFR | 0.016-0.07 | μM |
| (sensitive) | | | | |

*N/R = not reported in scientific literature

### Example 2

### Identification of Differentially Expressed microRNA Candidates Controlling Erlotinib Resistance

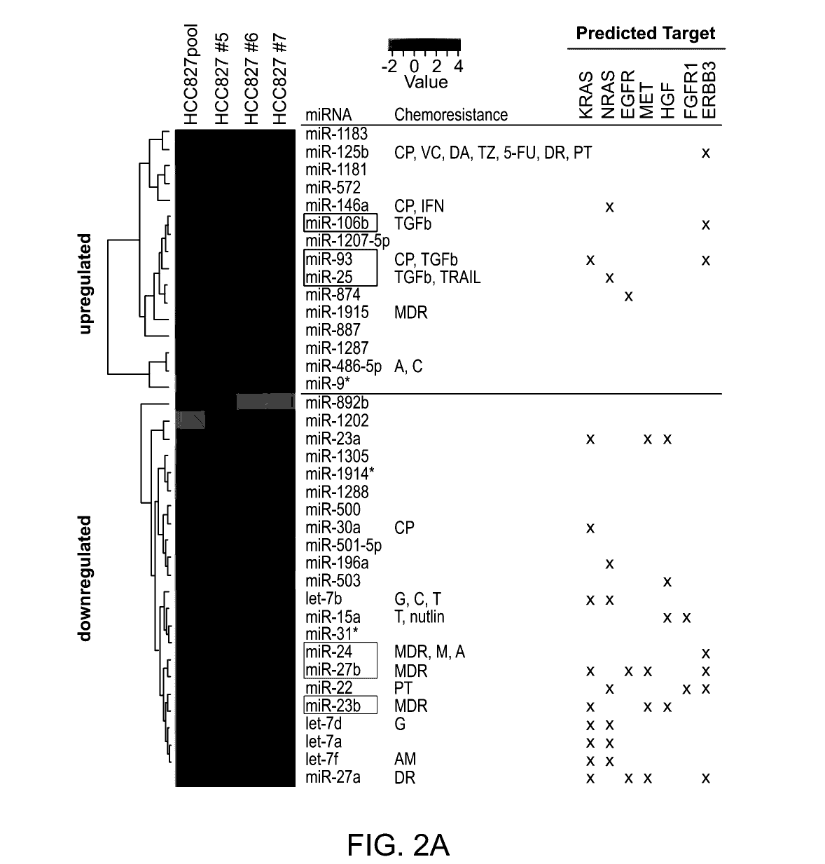

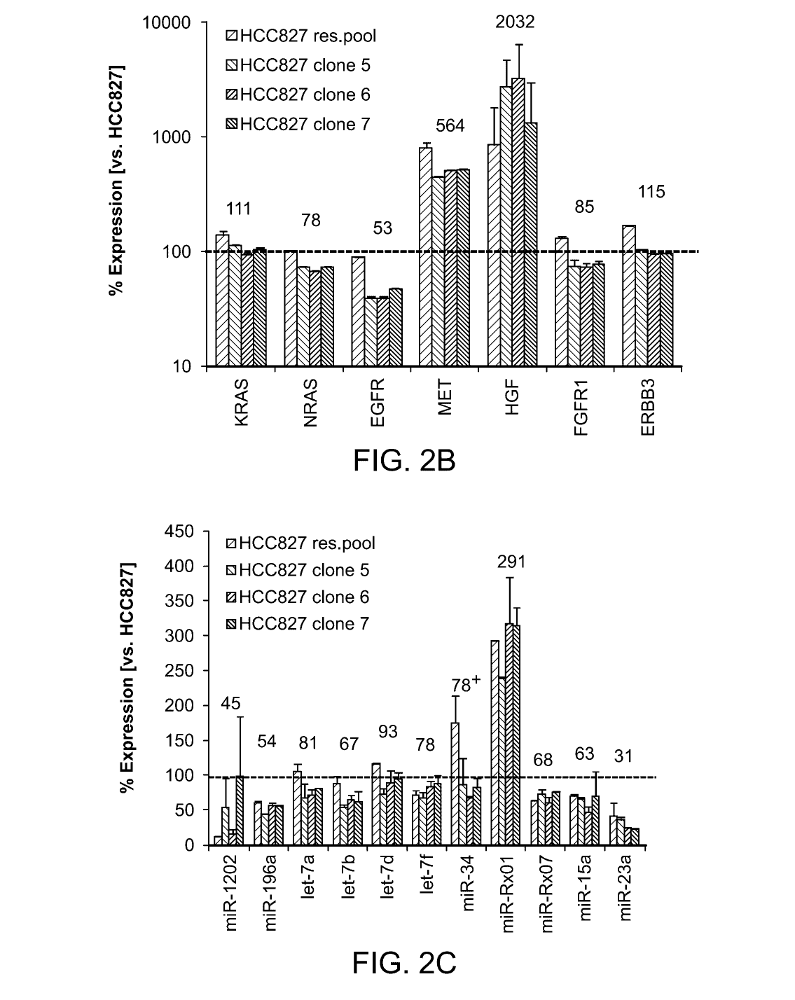

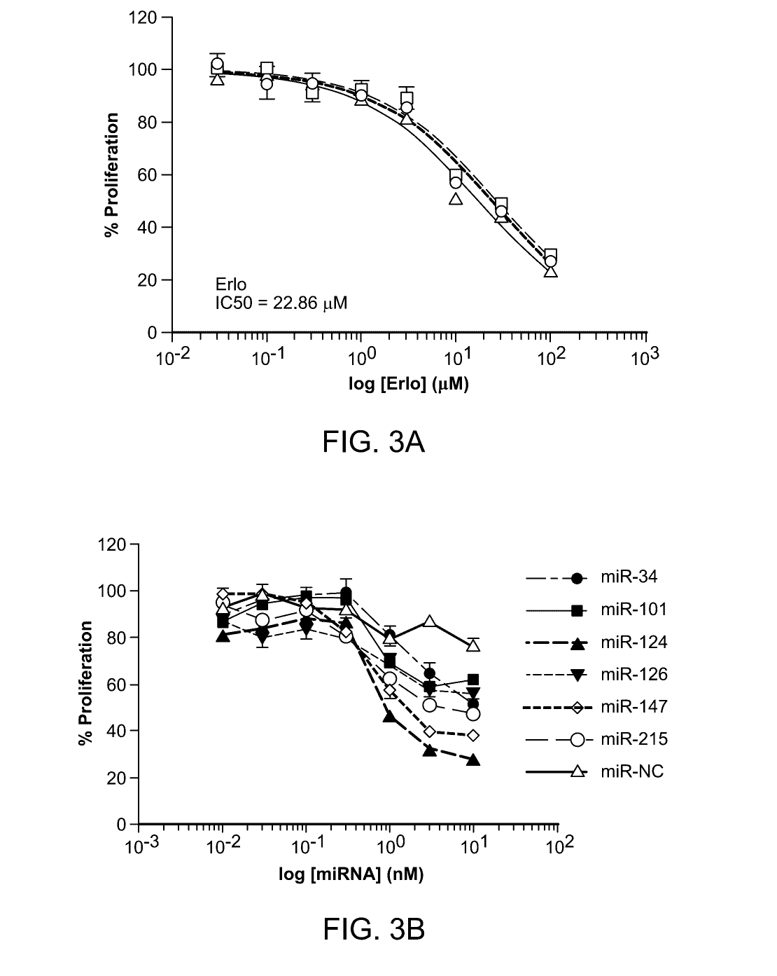

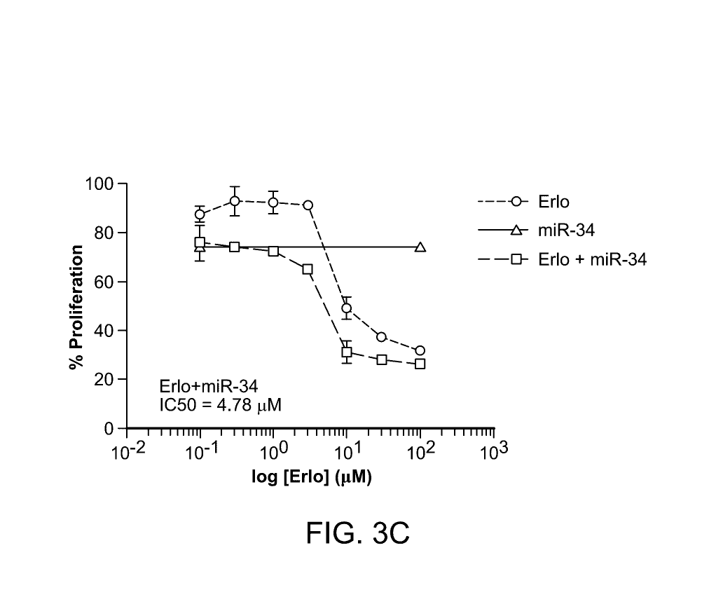

All four cell lines, as well as the parental HCC827 line were used for RNA extraction and subjected to mRNA (Affymetrix HG-U133 Plus 2.0) and miRNA (Agilent/Sanger12_0) array analysis. Unexpectedly, relatively few mRNAs were differentially expressed between resistant and parental lines (data not shown). In contrast, expression levels of miRNAs were significantly altered. A comparison of miRNA expression between the resistant cells and the parental line showed that clone #7 is most closely related to HCC827 (R²=0.9347), and the resistant pool is the least related line (R²=0.8308). This is in accord with the hypothesis that the pool arose from multiple clones. Unsupervised clustering of miRNAs identified 15 up-regulated and 23 down-regulated miRNAs across all resistant HCC827 cells when compared to the parental line (FIG. 2A). miRNAs that are encoded in a gene cluster and expressed as polycistronic transcripts, miR-106b˜93˜25 and miR-24˜27b˜23b, are all found to be up- or downregulated, respectively. This suggests that genetic mechanisms contribute to the differential expression of miRNAs in erlotinib-resistant cells. Many of the differentially expressed miRNAs have previously been associated with resistance to other chemotherapies—for instance, upregulated miRNAs in erlotinib-resistance HCC827 cells contribute to resistance to conventional, and downregulated miRNAs suppress chemoresistance. Two miRNAs (let-7b, miR-486) have been implicated in resistance to cetuximab, a monoclonal antibody against EGFR. The involvement in erlotinib resistance is novel for all miRNAs. A search for gene products predicted to be repressed by these miRNAs revealed that miRNAs downregulated in erlotinib-resistant cells have a higher propensity to repress known erlotinib resistance genes, including RAS, EGFR, MET and HGF. Quantitative reverse-transcriptase PCR (qRT-PCR) showed that both MET and HGF were highly overexpressed in all erlotinib-resistant cell lines. This is consistent with previous reports demonstrating a role for the HGF/MET axis in acquired erlotinib resistance. MET and HGF overexpression might be the result of gene amplification as previously reported or, alternatively, a loss of miRNA expression that suppress these genes as suggested by our data set (subject of further investigation). Appendix A provides quantitative data underlying FIG. 2A.

### Example 3

### Combinatorial Effect of Erlotinib and Synthetic Oligonucleotides

Lung carcinoma cell lines used in the combination studies included cell lines resistant (H1299, H460, HCC827, all resistant) or sensitive (HCC827 parental) to erlotinib. The main aim of the combination was to achieve an enhanced therapeutic effect of erlotinib (decreased IC₅₀) and to reduce the dose and toxicity of erlotinib. The evaluation of the combinatorial work was performed following the “Fixed Concentration Model” (Fiebig, H. H., Combination Studies). The cytotoxic compound A (erlotinib) is tested at 7-8 concentrations, and compound B (miRNA) at one weak concentration. Drug or miRNA effects on cellular proliferation were assessed using AlamarBlue assay (Invitrogen, Carlsbad, Calif.). IC₅₀ values of erlotinib alone and in the combinations were calculated using the GraphPad software.

First, IC₅₀ values of erlotinib alone or miRNAs alone were determined in the cells. miRNAs were reverse transfected at fixed, weak concentration (˜IC₂₅). MicroRNA sequences used were as shown in Table 1. A scrambled sequence was used a negative control. Then, the cells were treated with erlotinib in a serial dilution. Cell proliferation inhibition was analyzed 3 days post drug treatment by AlarmaBlue assay. IC₅₀ values of erlotinib combined with miRNA was determined using the GraphPad software. The combinatorial effect was evaluated by the visual inspection of the dose response curve and a shift of the IC₅₀ value. The IC₅₀ results for erlotinib alone or in combination with each of the six tested miRNAs are reported in Table 4 respectively.

TABLE 4

| | RESISTANT | | | | | | SENSITIVE | |
|---|---|---|---|---|---|---|---|---|
| | H1299 | | H460 | | HCC827^{res.pool} | | HCC827 | |
| miRNA | IC₅₀ | P | IC₅₀ | P | IC₅₀ | P | IC₅₀ | P |
| Erlotinib | 21.5 (±5.7) | | 26.3 (±9.5) | | 77.6 (±73.4) | | 0.22 (±0.22) | |
| Erlotinib + miR-NC | 15.8 (±7.5) | n.s. | 25.3 (±8.1) | n.s. | 64.6 (±46.2) | n.s. | 0.24 (±0.25) | n.s. |
| Erlotinib + miR-34 | 4.6 (±0.3) | <0.01 | 10.6 (±2.1) | 0.055 | 2.7 (±3.2) | <0.01 | 0.03 (±0.03) | <0.01 |
| Erlotinib + miR-126 | 2.4 (±1.9) | <0.01 | 8.1 (±6.0) | <0.01 | 4.3 (±5.5) | <0.01 | 0.05 (±0.07) | <0.01 |
| Erlotinib + miR-124 | 1.0 (±1.1) | <0.01 | 6.4 (±1.5) | <0.05 | 10.2 (±13.3) | <0.01 | 0.002 (±0.002) | <0.01 |
| Erlotinib + miR-147 | 3.4 (±3.1) | <0.01 | 12.6 (±5.3) | n.s. | 0.8 (±0.8) | <0.01 | 0.01 (±0.01) | <0.01 |
| Erlotinib + miR-215 | 1.1 (±0.8) | <0.01 | 9.2 (±0.7) | <0.05 | 3.1 (±1.3) | 0.053 | 0.01 (±0.01) | <0.01 |
| Erlotinib + miR-101 | 8.7 (±2.6) | n.s. | 38.2 (±12.5) | n.s. | 25.1 (±23.7) | n.s. | 0.12 (±0.08) | n.s. |

### Example 4

### In Vivo Efficacy Assessment for Erlotinib and miRNAs

To test effects of the erlotinib/synthetic oligonucleotide combinations in vivo, a tumor mouse model is used that, for instance, is based on orthotopic xenografts that stably express a luciferase reporter gene. A typical efficacy study includes 8 animals per group. Next to erlotinib/miRNA combinations, other study groups include erlotinib alone, miRNA alone, as well as erlotinib/miR-NC and no-treatment controls. When tumor lesions in the lung become apparent through IVIS imaging, miRNA treatment is started. miRNAs are administered intravenously every other day complexed in the nanoparticles at a moderately effective dose to allow the detection of erlotinib enhancement (1-10 mg/kg). Erlotinib will be given daily by gavage at a dose of/day which has shown to be well tolerated in mice. Treatment durations are 2-4 weeks, or until control mice become moribund whichever comes first. Animals are monitored closely to detect signs of toxicity. Upon sacrifice, lungs and lung tumor tissues are collected and subjected to histopathological analysis (H&E; ki67 and casp3 IHC if justified). RNA are extracted from normal lung, lung tumors, spleen and whole blood to measure concentrations of miRNA mimics by qRT-PCR. In addition, tumor samples are used to test for knock-down of direct/validated miRNA targets (qRT-PCR). The level of metastases in major organs can be assessed, either by H&E and a human-specific IHC stain (STEM121, StemCells, Inc.).

It is expected that the erlotinib/miRNA combinations show better in vivo efficacy than erlotinib alone with a concurrent repression of known miRNA targets in the tumor tissue. It is also expected that animals treated with erlotinib/miRNA combos are less likely to develop metastases and show improved survival.

### Example 5

### In Vitro Efficacy Assessment for EGFR-TKI and microRNA

Introduction

This example investigates the relationship of miR-34a and erlotinib and the therapeutic activity of the combination in NSCLC cells with primary and acquired erlotinib resistance. The drug combination was also tested in a panel of hepatocellular carcinoma cells (HCC), a cancer type known to be refractory to erlotinib. Using multiple analytical approaches, drug-induced inhibition of cancer cell proliferation was determined to reveal additive, antagonistic or synergistic effects. The data show a strong synergistic interaction between erlotinib and miR-34a mimics in all cancer cells tested. Synergy was observed across a range of dose levels and drug ratios, reducing IC₅₀ dose requirements for erlotinib and miR-34a by up to 46-fold and 13-fold, respectively. Maximal synergy was detected at dosages that provide a high level of cancer cell inhibition beyond the one that is induced by the single agents alone and, thus, is of clinical relevance. The data shows that a majority of NSCLC and other cancers previously not suited for EGFR-TKI therapy prove sensitive to the drug when used in combination with a micro RNA based therapy.

Materials and Methods

Cell lines: Human non-small cell lung cancer (NSCLC) cell lines A549, H460, H1299, H226, HCC827 parental and HCC827^{res} were used to assess the combinatorial effects of micro RNA and EGFR-TKIs. The particular cell lines were selected based on the high IC₅₀ values of EGFR-TKIs in these cells, their oncogenic properties and susceptibility to miRNAs. These cell lines are either resistant (A549, H460, H1299, H226) or sensitive (HCC827). In addition, cell lines with acquired resistance were created by applying increased selective pressure of erlotinib over ten weeks, starting at an equivalent of IC₁₀ and ending at an IC₉₀ equivalent. As cellular proliferation exhibited normal doubling rates under IC₉₀ selection, the resistant cells were plated at a low dilution (HCC827) or high dilution to create near-pure, resistant clones (HCC827^{res}-#5, 6 and 7). To study effects in hepatocellular carcinoma (HCC) cells, Hep3B, Huh7, C3A and HepG2 were used. Huh7 cells were acquired from the Japanese Collection of Research Bioresources Cell Bank. All other parental cells were purchased from the American Type Culture Collection (ATCC, Manassas, Va.) and cultured according to the supplier's instructions.

RNA isolation and qRT-PCR: Total RNA from cell pellets was isolated using the mirVANA PARIS RNA isolation kit (Ambion, Austin, Tex.) following the manufacturer's instructions. RNA concentration was determined by absorbance measurement (A260) on a Nanodrop ND-1000 (Thermo Scientific, Wilmington, Del.). For the quantification of miRNA and mRNA by quantitative reverse-transcription polymerase chain reaction (qRT-PCR), we used commercially available reagents. The RNA was converted to cDNA using MMLV-RT (Invitrogen, Carlsbad, Calif.) under the following conditions: 4° C. for 15 m; 16° C. for 30 min; 42° C. for 30 m; 85° C. for 5 mm Following cDNA synthesis, qPCR was performed on 2 μL of cDNA on the ABI Prism 7900HT SDS (Applied Biosystems, Life Technologies, Foster City, Calif.) using Platinum Taq Polymerase (Invitrogen) under the following cycling conditions: 95° C. for 1 min (initial denature); then 50 cycles of 95° C. for 5 sec, 60° C. for 30 sec. TaqMan Gene Expression Assays and TaqMan MicroRNA Assays were used for expression analysis of mRNA and miRNA in all lung and liver cell lines. For miRNA expression, additions to the manufacturers' reagents include DMSO (final concentration of 6%) and tetramethylammoniumchloride (TMAC; final concentration of 50 mM in both RT and PCR) to improve the slope, linearity and sensitivity of the miRNA assays. Expression levels of both miRNA and mRNA were determined by relative quantitation to the HCC827 parental cell line. The raw Ct values of the miRNA and mRNA targets were normalized to selected housekeeping genes to create delta-Ct values, converted to linear space and then expressed as percentage expression.

miRNA and EGFR-TKI treatment: Erlotinib hydrochloride was purchased from LC Laboratories (Woburn, Mass.). Synthetic miR-34a and miR-NC mimics were manufactured by Life Technologies (Ambion, Austin, Tex.). To determine the IC₅₀ value of each drug alone, 2,000-3,000 cells per well were seeded in a 96-well plate format and treated with either erlotinib or miR-34a as follows. (i) miR-34a mimics were reverse-transfected in triplicates in a serial dilution (0.03-30 nM) using RNAiMax lipofectamine from Invitrogen. As controls, cells were also transfected with RNAiMax alone (mock) or in complex with a negative control miRNA mimic (miR-NC). Cells were incubated with AlamarBlue (Invitrogen) 4 days or 6 days post transfection to determine cellular proliferation of lung or liver cancer cells, respectively. Proliferation data were normalized to mock-transfected cells. (ii) Erlotinib, prepared as a 10 and 20 mM stock solution in dimethyl sulfoxide (DMSO), was added to cells one day after seeding at a final concentration ranging from 0.1 and 100 μM. Solvent alone (0.5% final DMSO in H226 and HCC827, 1% final DMSO in all other cell lines) was added to cells in separate wells as a control. Three days thereafter, cellular proliferation was measured by AlamarBlue and normalized to the solvent control.

Regression trendlines & IC₅₀ values: Linear and non-linear regression trendlines were generated using the CompuSyn (ComboSyn, Inc, Paramus, N.J.) and Graphpad (Prism) software, respectively. The non-linear trendlines provided a better fit for the actual data and were used to calculate IC₅₀, IC₂₅ and other drug concentrations (IC), although both software programs generated similar values.

Combination Effects Determined by the “Fixed Concentration” Method

The “Fixed Concentration” method was used for cell lines with acquired resistance (HCC827). Cells were reverse-transfected with miR-34a using the miRNA at a fixed, weak concentration (˜IC₂₅) as described above. The following day, cells were treated with erlotinib in a serial dilution (0.01-100 μM). Cell proliferation inhibition was analyzed 3 days later by AlamarBlue. To measure the effects of the single agents and to correct for effects potentially contributed by lipid carrier or vehicle, cells were also treated with miR-34a in combination with solvent (0.5% DMSO in HCC827^{res}, 1% DMSO in all other cell lines) or erlotinib in combination with mock-transfection. All proliferation data was normalized to mock-transfected cells treated with solvent (DMSO). The combinatorial effect was evaluated by a visual inspection of the erlotinib dose-response curve and a shift of the IC₅₀ value in the presence or absence of miR-34a (graphed and calculated using Graphpad).

Combination Effects Determined by the “Fixed Ratio” Method

Cells were treated with 7 concentrations of erlotinib each in combination with 7 concentrations of miR-34a. Each drug was used at a concentration approximately equal to its IC₅₀ and at concentrations within 2.5-fold (NSCLC) or 2-fold (HCC) increments above or below. This matrix yielded a total of 49 different combinations representing 13 different ratios. Each drug was also used alone at these concentrations. miR-34a and erlotinib were added as described above, and cellular proliferation was determined by AlamarBlue. Each data point was performed in triplicates.

Calculation of Combination Index (CI) Values

CI values based on Loewe's additivity model were determined to assess the nature of drug-drug interactions that can be additive (CI=1), antagonistic (CI>1), or synergistic (CI<1) for various drug-drug concentrations and effect levels (Fa, fraction affected; inhibition of cancer cell proliferation). Both linear regression and nonlinear regression trendlines were used to calculate and compare CI values. CI values based on linear regression analysis was done using the CompuSyn software (ComboSyn Inc., Paramus, N.J.), following the method by Chou et al., whereby the hyperbolic and sigmoidal dose-effect curves are transformed into a linear form (Chou T C (2010) Drug combination studies and their synergy quantification using the Chou-Talalay method. Cancer Res 70: 440-6, instructions also available at ComboSyn, Inc., www.combosyn.com). CI values derived from non-linear regression trendlines were calculated using Equation 1 in which C_{A,x} and C_{B,x} are the concentrations of drug A and drug B in the combination to produce effect X (Fa). IC_{x,A} and IC_{x,B} are the concentrations of drug A and drug B used as a single agent to produce that same effect.

$$CI = \frac{{C}_{A , x}}{{IC}_{x , A}} + \frac{{C}_{B , x}}{{IC}_{x , B}} \qquad Equation 1$$

Drug concentrations required in Equation 1 to determine CI values (C_{A,x}, C_{B,x}, IC_{x,A} and IC_{x,B}) were calculated using the Hill equation (Equation 2), IC₅₀ and Hill slope value (n) derived from non-linear regression trendlines (Graphpad).

$$E = {E}_{max} \times \frac{{C}^{n}}{{IC}_{50}^{n} + {C}^{n}} \qquad Equation 2$$

Isobolograms

To describe the dose-dependent interaction of erlotinib and miR-34a, isobolograms at effect levels of 50% and 80% inhibition of cancer cell proliferation were created. Since the single agents—alone or in combination—usually reached 50% cancer cell inhibition, the 50% isobologram provided an actual comparison of the single use vs. the combination. The 80% isobologram was used to illustrate the utility of the combination at a high effect level that have practical implications in oncology. In each of these, additivity was determined by extrapolating the dose requirements for each drug in combination from its single use (IC₅₀, IC₈₀). Data points above or below the line of additivity indicate antagonism or synergy, respectively. For all 49 combinations, drug concentrations required in the combination were compared to those of the single agents alone to reach the same effect and expressed as a fold change (dose reduction index, DRI).

Curve Shift Analysis

To allow a direct comparison of the dose-response curves and to identify synergistic drug-drug interaction, non-linear regression trendlines of each drug alone or of the combination (IC₅₀:IC₅₀ ratio or other ratios where indicated) were normalized to its own IC₅₀ value and referred to as IC₅₀ equivalents (IC₅₀ eq). IC₅₀ equivalents of the combination were calculated using Equation 3 and described in Zhao L, Au J L Wientjes M G (2010) Comparison of methods for evaluating drug-drug interaction. Front Biosci (Elite Ed) 2: 241-9. Data of the single agents and in combination were graphed in the same diagram to illustrate lower drug concentrations required to achieve any given effect relative to the single agents. This is represented in a left-shift of the dose-response curve and indicates synergy. Id.

$${IC}_{50 eq} = \frac{{C}_{A , x}}{{IC}_{50 , A}} + \frac{{C}_{B , x}}{{IC}_{50 , B}} \qquad Equation 3$$

Statistical Analysis

Statistical analysis was done using the Excel (Microsoft), CompuSyn and Graphpad software. Averages and standard deviations were calculated from triplicate experiments. Goodness of fit of linear and non-linear regression trendlines was described by R (CompuSyn) and R² (Graphpad) values, respectively, and were >0.9 for most cell lines except H226 and HepG2 cells due to limiting drug insensitivity.

Results

miR-34a Restores Sensitivity to Erlotinib in Non-Small Cell Lung Cancer Cells

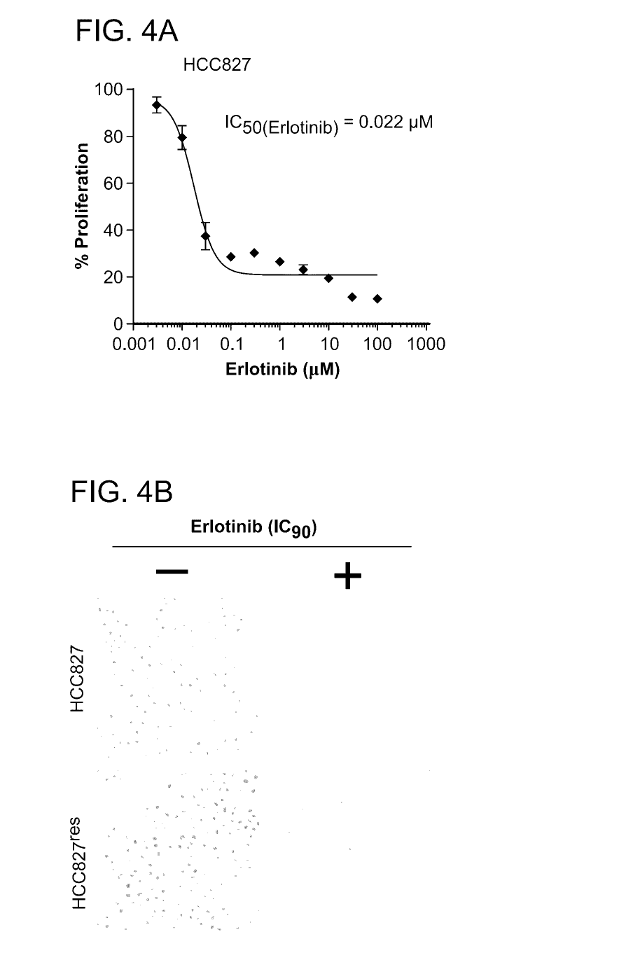

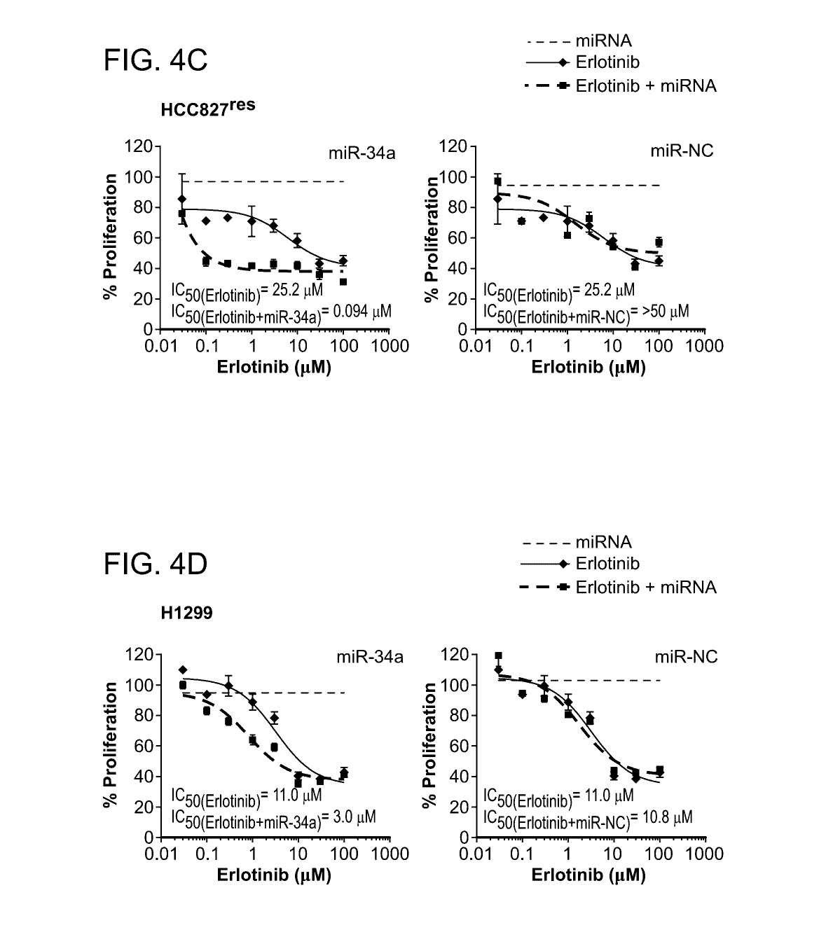

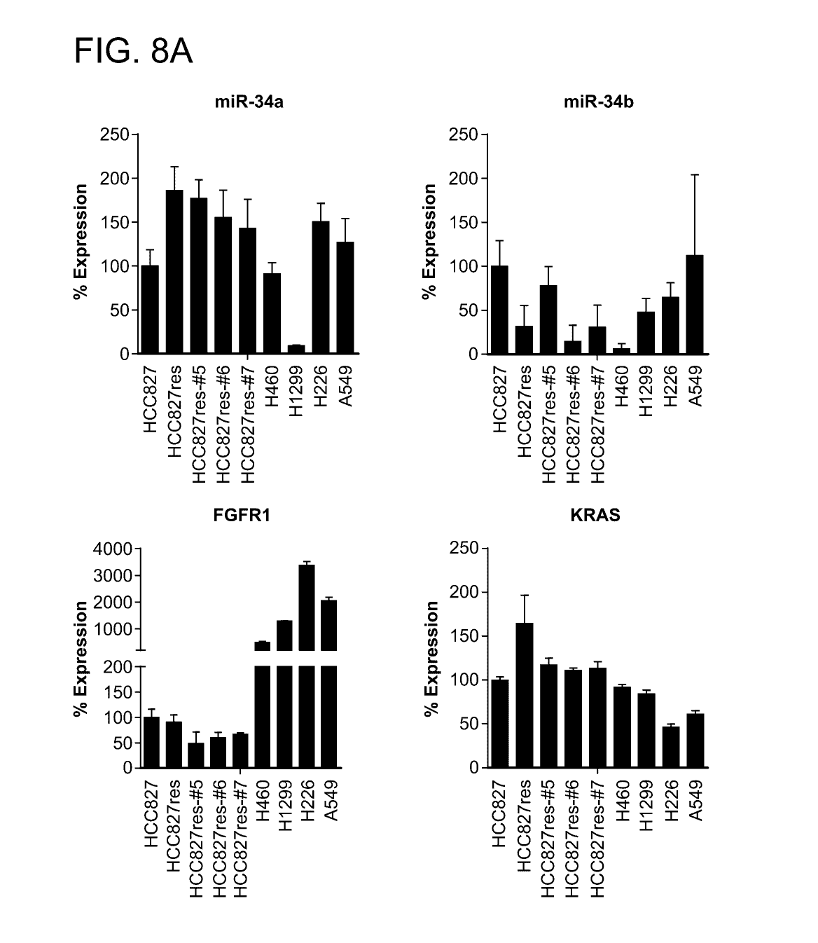

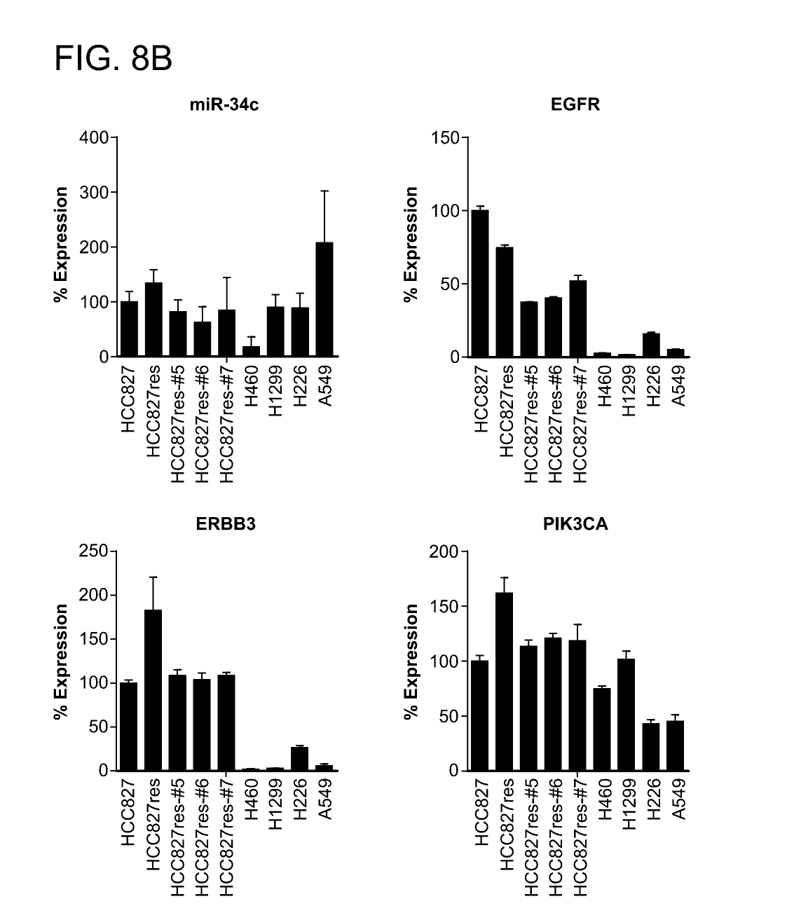

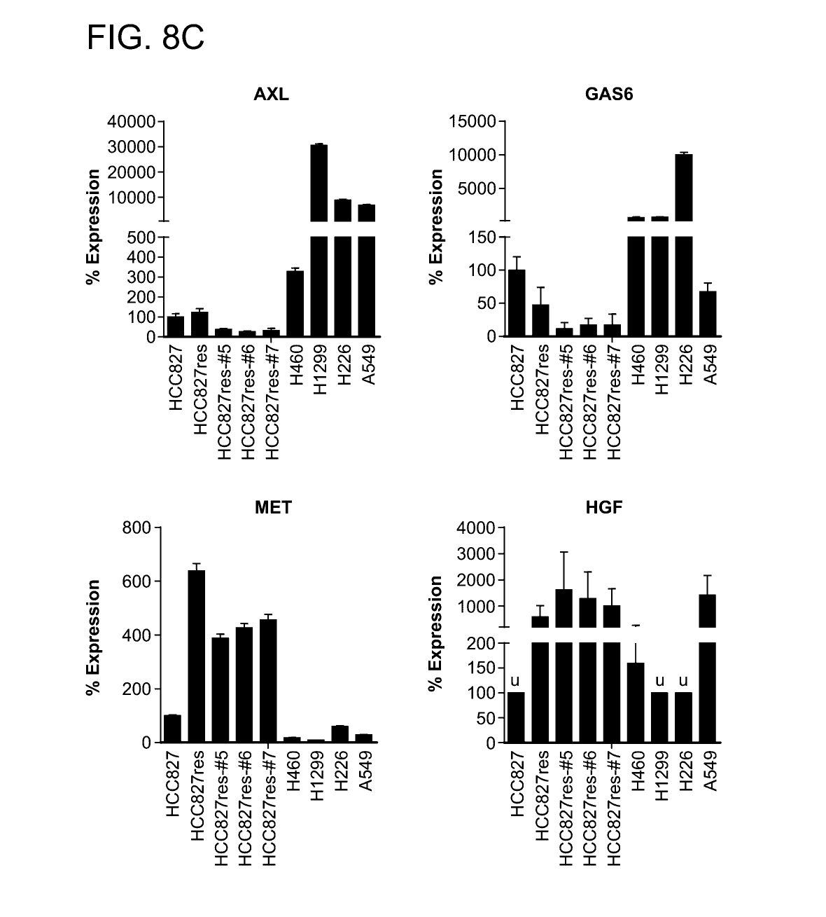

To study drug resistance in cells with acquired resistance, we used HCC827 cells that express an activating EGFR mutation (deletion of exon 19 resulting in deletion of amino acids 745-750). HCC827 are highly sensitive to erlotinib with an IC₅₀ value of 0.022 μM (FIG. 4A). Erlotinib-resistant cell lines were developed by exposing the parental HCC827 cells to increasing erlotinib concentrations over the course of 10 weeks until the culture showed no signs of growth inhibition at a concentration that is equivalent to IC₉₀ in the parental cell line (FIG. 4B). During this process, individual cell clones (HCC827^{res}-#5, #6, #7) as well as a pool of resistant cells (HCC827) were propagated. Total RNA was isolated and probed by quantitative PCR for levels of miR-34 family members and genes known to induce resistance. HCC827 cells resistant to erlotinib showed increased mRNA levels of MET and its ligand HGF that presumably function to bypass EGFR signaling (FIGS. 8A-C). In contrast, expression levels of other genes also associated with resistance, such as AXL, GAS6, KRAS, FGFR1, ERBB3, PIK3CA and EGFR itself, were not elevated. Levels of miR-34b/c family members were reduced in several of the resistant HCC827 cells (FIGS. 8A-C). Interestingly, miR-34a was not reduced in erlotinib-resistant HCC827 cells suggesting that miR-34a does not play a causal role in the onset of resistance in these cells which can occur independently of miR-34 by amplification of the MET gene.

Since both MET and AXL are directly repressed by miR-34, and because inhibition of AXL can antagonize erlotinib resistance, the introduction of synthetic miR-34 mimics may restore erlotinib sensitivity. To explore this possibility, HCC827^{res} cells were exposed to increasing erlotinib concentrations, ranging from 0.03-100 μM, either in the absence or presence of miR-34a used at a fixed, weak concentration (0.3 nM). The effects of erlotinib were expected to be concentration-dependent, such that erlotinib in combination with miR-34a produced lower IC₅₀ values relative to erlotinib alone. As shown in FIG. 4C, erlotinib was not very potent in HCC827^{res} cells (IC₅₀=25.2 μM). However, when used in combination with miR-34a, the erlotinib IC₅₀ value decreased to 0.094 μM. This result shows that adding a small amount of miR-34a is capable of restoring erlotinib sensitivity that is similar to the one of parental HCC827 cells. The effects were specific to the miR-34a sequence as the addition of a negative control miRNA (miR-NC) did not improve the potency of erlotinib (FIG. 4C). Thus, the data generated in HCC827 cells indicate that miR-34a can sensitize cancer cells with acquired erlotinib resistance.

To determine whether the miRNA can also counteract primary resistance mechanisms, we used H1299 cells that have mutations in the NRAS and TP53 genes. In these cells, erlotinib produced an IC₅₀ value of 11.0 μM (FIG. 4D). In combination with 0.3 nM miR-34a, the erlotinib dose-response curve shifted along the x-axis, indicating an approximately 4-fold lower IC₅₀ value (3.0 μM). This result is in contrast to miR-NC that did not alter the potency of erlotinib, and suggests that miR-34a sensitizes non-small lung cancer cells with both acquired as well as primary resistance.

miR-34a and Erlotinib Synergize in Non-Small Cell Lung Cancer Cells

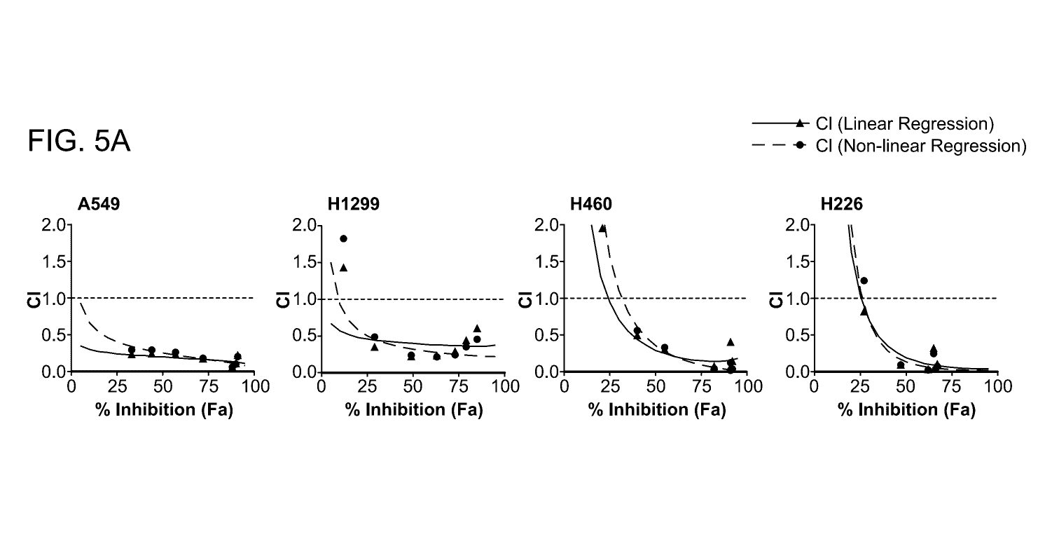

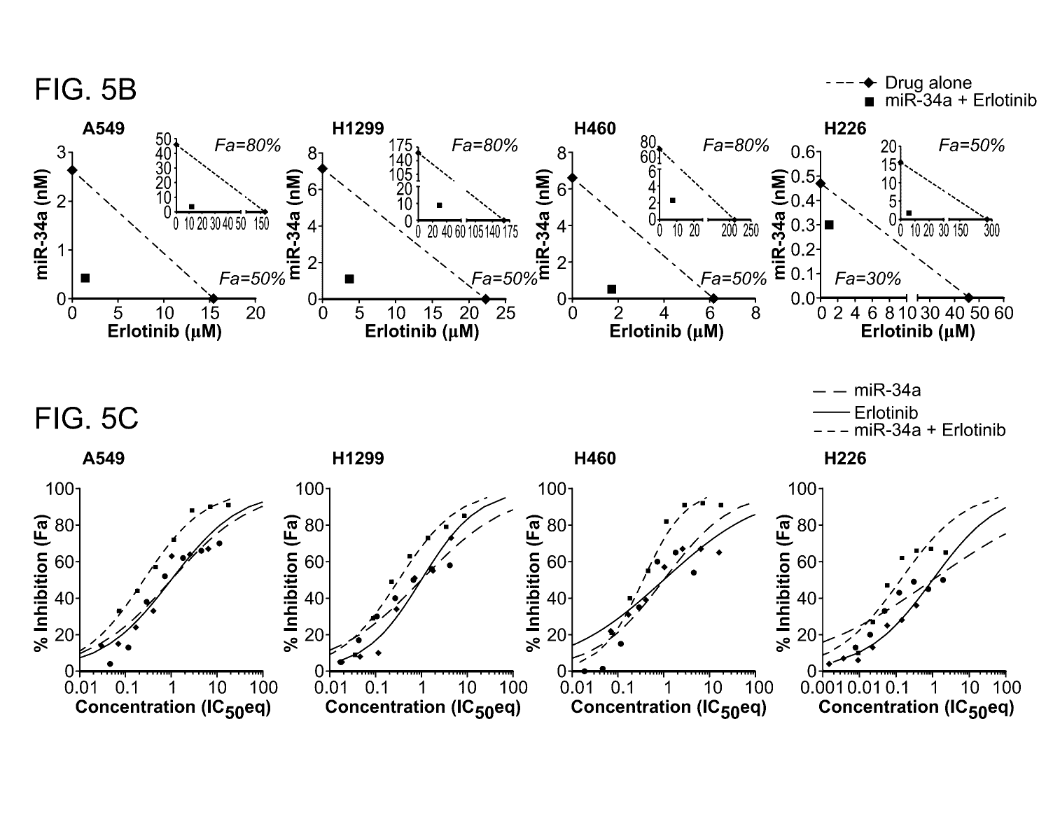

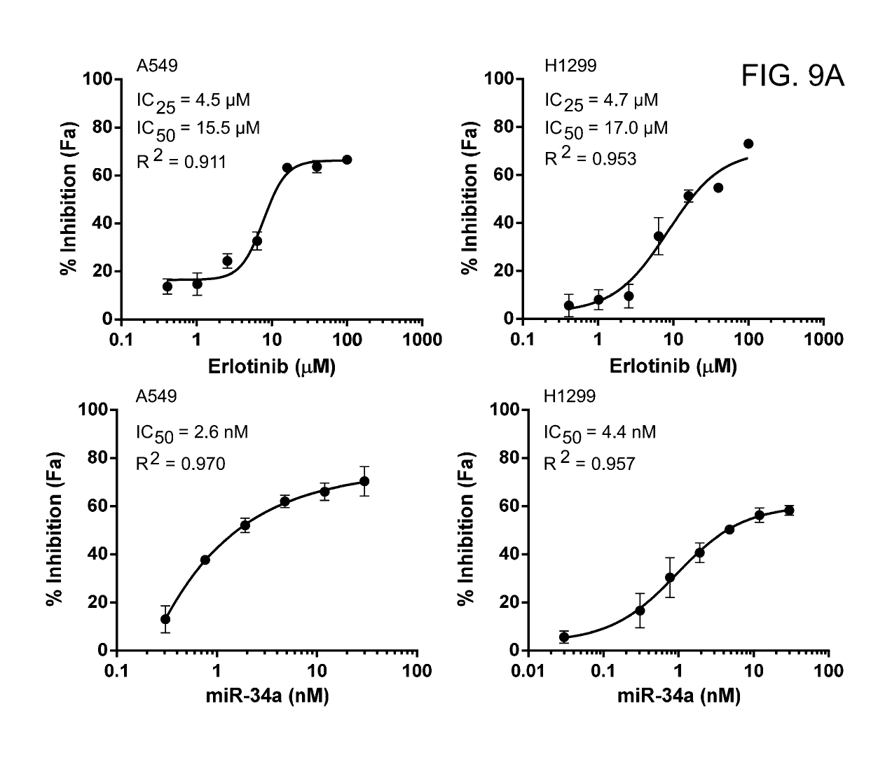

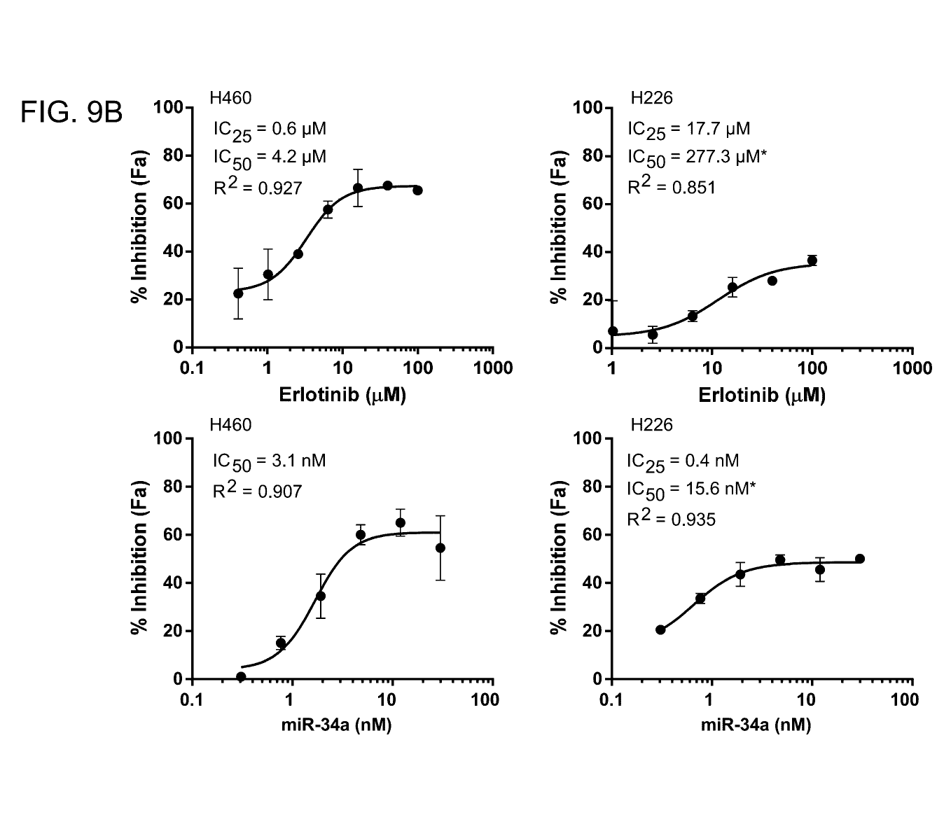

The shift of the erlotinib IC₅₀ value demonstrated how a fixed miR-34a concentration can improve the potency of erlotinib. However, this model, also known as “Fixed-Concentration-Model”, does not allow the assessment of synergy. To investigate whether both drugs can enhance each other, we employed the “Fixed-Ratio-Model” that is based on Loewe's concept of additivity (Chou T C (2010) Drug combination studies and their synergy quantification using the Chou-Talalay method. Cancer Res 70: 440-6. Tallarida R J (2001) Drug synergism: its detection and applications. J Pharmacol Exp Ther 298: 865-72. Tallarida R J (2006) An overview of drug combination analysis with isobolograms. J Pharmacol Exp Ther 319: 1-7.) In this model, combination index (CI) values are calculated based on the slope and IC₅₀ value of each dose-response curve (drug alone or in combination) and define whether the drug-drug interactions are synergistic (CI<1), additive (CI=1), or antagonistic (CI>1). Since the accuracy of the CI values depends on the fit of the dose-response curve trendline, CI values were calculated by two methods using either linear or non-linear regression trendlines (see Materials and Methods). Four erlotinib-resistant cell lines were used, all of which differ in their genetic make-up: A549 (mutations in KRAS, STK11, CDKN2A), H460 (mutations in KRAS, STK11, CDKN2A, PIK3CA), H1299 (mutations in NRAS, TP53), and H226 (mutations in CDKN2A) [37]. A qRT-PCR analysis showed a marked increase of AXL, GAS6 and FGFR1 mRNA levels in these cells relative to erlotinib-sensitive HCC827 cells, further providing an explanation for erlotinib resistance (FIGS. 8A-C). Levels of miR-34 were significantly reduced in H1299 and H460 cells. In a first step, erlotinib or miR-34a were added to cells in a serial dilution to determine IC₅₀ values of each drug alone. For erlotinib, these ranged between 4.2 and >50 μM (FIGS. 9A-B). The IC₅₀ values of miR-34a ranged from 0.4 to 15.6 nM. Neither drug was capable of 100% cancer cell inhibition, nor did the maximal activity of either drug exceed 75%. Erlotinib and miR-34a were least effective in H226 cells, yielding theoretical IC₅₀ values as a result of an extrapolation of the dose-response curve. In a second step, each drug was combined at a concentration equal to its own approximate IC₅₀ value, as well as at multiples thereof above and below (fixed ratio). As controls, each drug was used at these concentrations alone. Both linear and non-linear regression models produced CI values that are well below 1.0 in all cell lines tested indicating strong synergy (FIG. 5A). CI values we considered relevant are those below 0.6. In most cell lines, synergy was observed at higher dose levels and at higher magnitude of cancer cell inhibition. This is critical because a practical application of the drug combination calls for synergy at maximal cancer cell inhibition (75% inhibition or greater). In general, the non-linear regression trendline provided a better fit for the actual data, although both models generated similar results.

Next, we generated isobolograms and determined the dose requirements for each drug at 50% and 80% cancer cell inhibition as a read-out for synergy. The 50% effect level was chosen because the potency of a drug is frequently assessed at its IC₅₀ and because in our studies each drug alone was capable of inhibiting most cancer cells by 50%, allowing a comparison of each drug alone with the combination within the range of actual data. The 80% effect level was chosen because it is important to demonstrate synergy at high inhibitory activity for oncology applications. Although the concentrations of each drug alone to achieve 80% inhibition are based on an extrapolation of the dose-response curve and are theoretical in nature, the miR-34a-erlotinib combination readily achieved 80% inhibition or greater and is within the range of actual data. Since the two drugs by themselves were not very effective in H226 cells, isobolograms at 30% and 50% inhibition were created for H226 data. As shown in FIG. 5B, the isobole of the combination was well below the additive isobole for every cell line and effect level indicating strong synergy. The dose requirement for erlotinib decreased to 2 μM or less in most cell lines to achieve 50% inhibition, reducing the dose by 4- to 46-fold. Likewise, the required concentration of miR-34a was also substantially less in the combination relative to miR-34a alone, reducing its dose by 7- to 13-fold. This reduction in dose level, also referred to as dose reduction index (DRI), was markedly evident at 80% inhibition at which the dose requirements were reduced by up to 28-fold (erlotinib) and 33-fold (miR-34a).

Third, we performed curve-shift analyses whereby the concentration of each drug has been normalized to its own IC₅₀ value (Zhao L, Au J L Wientjes M G (2010) Comparison of methods for evaluating drug-drug interaction. Front Biosci (Elite Ed) 2: 241-9.). This conversion of drug concentrations into IC₅₀ equivalents (IC₅₀ eq) allows a direct comparison of each dose-response curve from the single agents and the combination. Trendlines were generated and span effect levels from 0-100% inhibition. The slope of the trendline indicates drug potency, and the maximal activity can be gauged from actual data points. Synergy is identified when IC₅₀ equivalents of the combination are lower to achieve any given effect relative to the single agents. Id. This is visually indicated by a left-shift of the combination trendline. As seen in FIGS. 8A-C, the combination is well separated from the single agents indicating synergy. In H460 and H226 cells, the IC₅₀ equivalents of the combination are greater at low effect levels (0-25%) and lower at effect levels above 30% compared to those of the single agents. This observation agrees with data from CI plots showing antagonism below 25% inhibition and synergy above 25% inhibition in these cells (FIG. 4A). Thus, the analysis reveals synergistic effects for drug concentrations that induce a high level of cancer cell inhibition. A benefit for the combination is further demonstrated by the fact that the actual level of inhibition is greater for the combination relative to the single agents—the maximal activity of the single drugs is no greater than 75% and can be extended beyond 90% when used in combination.

Various Ratios of Erlotinib and miR-34a Cooperate Synergistically

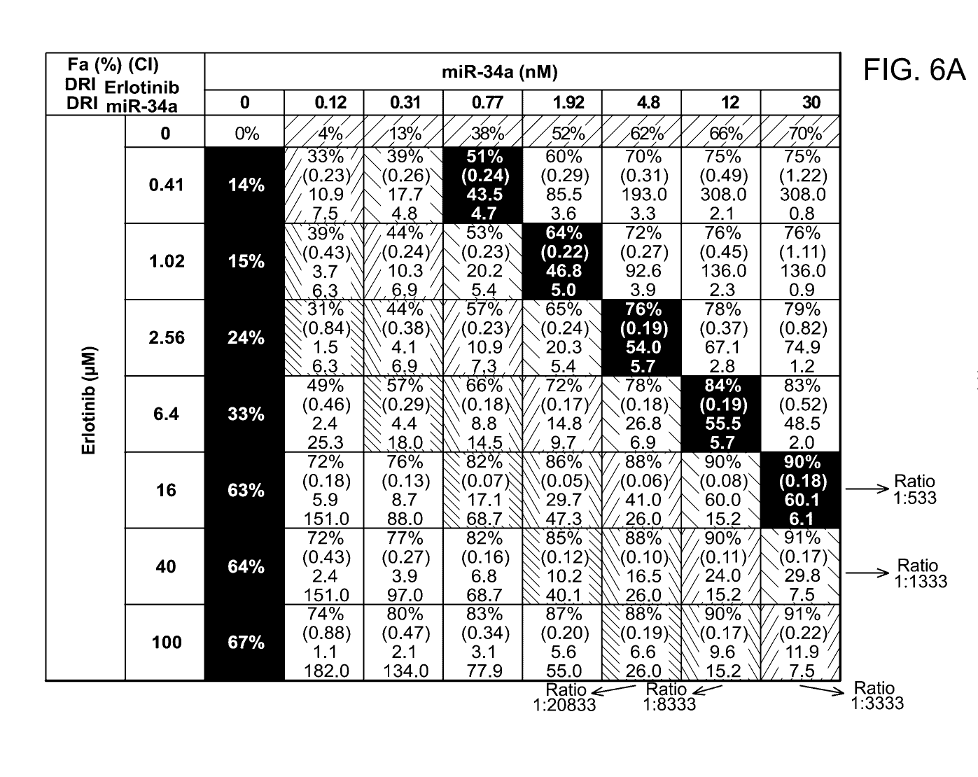

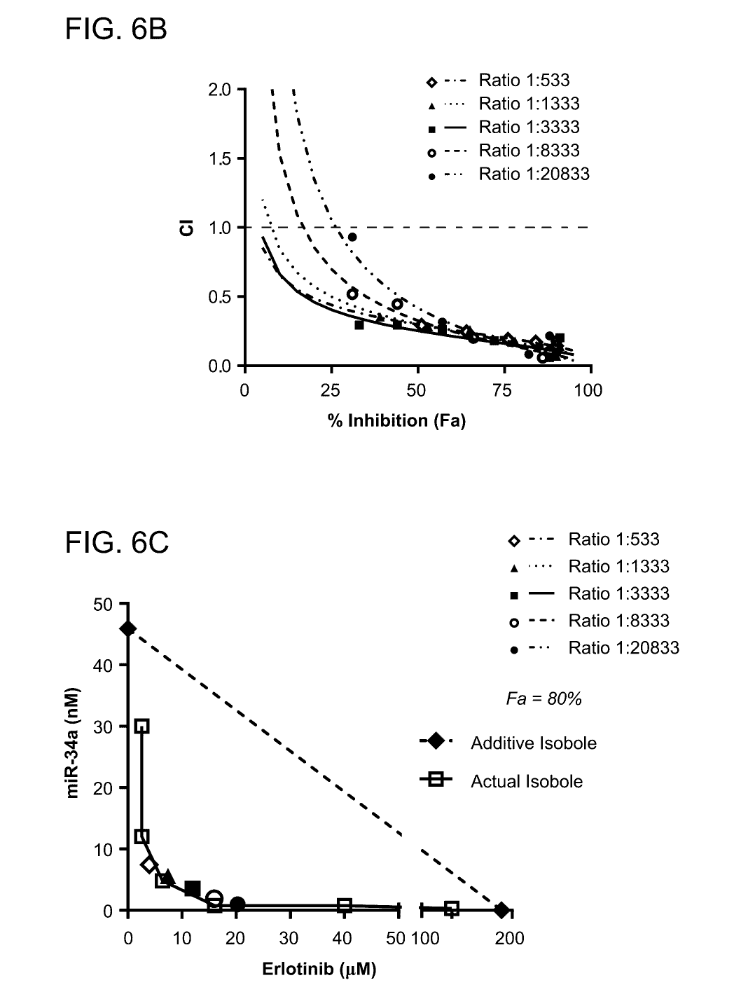

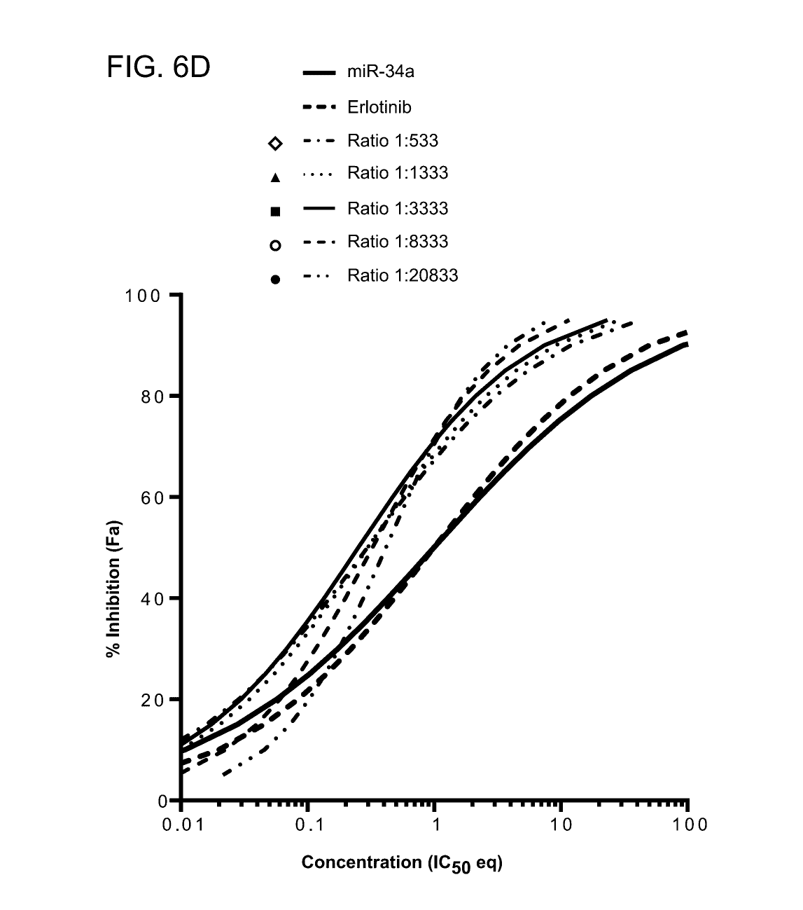

Our analysis suggests that erlotinib and miR-34a synergize when the two drugs are combined at a ratio derived from their IC₅₀ values. Because drug-drug interactions can change depending on the relative amounts, we explored the effects of multiple erlotinib-miR-34a ratios by combining erlotinib at concentrations from 0.41-100 μM with miR-34a at concentrations from 0.12-30 nM. Drug doses were increased in 2.5-fold increments, and each drug was also used alone as controls. This matrix yielded 49 drug combinations representing 13 different drug ratios (FIG. 6A). Levels of cancer cell inhibition, CI and DRI values were determined for each combination and graphed in CI plots, isobolograms and curve-shift diagrams. In this example, we focused on combinations in which miR-34a and erlotinib were added in an IC₅₀:IC₅₀ ratio (molar ratio 1:3333) and the following molar-based ratios: 1:533, 1:1333, 1:8333 and 1:208333.

Calculated CI values predict that erlotinib and miR-34a combined at all of these ratios provide strong synergy (FIG. 6B). At effect levels greater than 75% inhibition, CI values were below 0.2. The ratios that contained higher amounts of erlotinib provided lower synergy at effect levels below ˜75% and were slightly superior at effect levels above 75% inhibition. Similarly, the isobologram indicates strong synergy for various erlotinib-miR-34a ratios (FIG. 6C). Actual data points demonstrate that 30 nM miR-34a or 100 μM erlotinib are required to induce ˜80% cancer cell inhibition when used as single agents. In contrast, the required dose levels of erlotinib in the combination were substantially decreased as miR-34a amounts were increased. For instance, merely 2.56 μM erlotinib was needed to induce ˜80% inhibition when used with 12 nM miR-34a, thereby reducing the dose requirement of erlotinib by ˜40-fold. Further evidence for the synergistic action of these ratios comes from curve-shift analyses that reveal much lower IC₅₀ equivalents of the combination compared with IC₅₀ values of the single agents alone (FIG. 6D). The IC₅₀ eq data correlate with CI data showing dose-dependent degrees of synergy among various ratios: low ratios show lower synergy at low effect levels which is reversed at high levels of cancer cell inhibition.

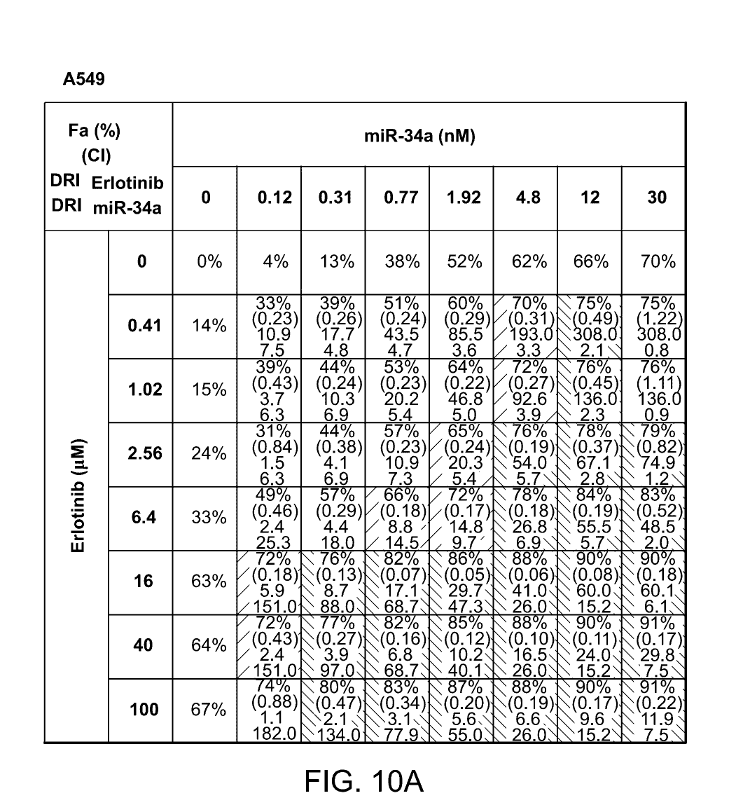

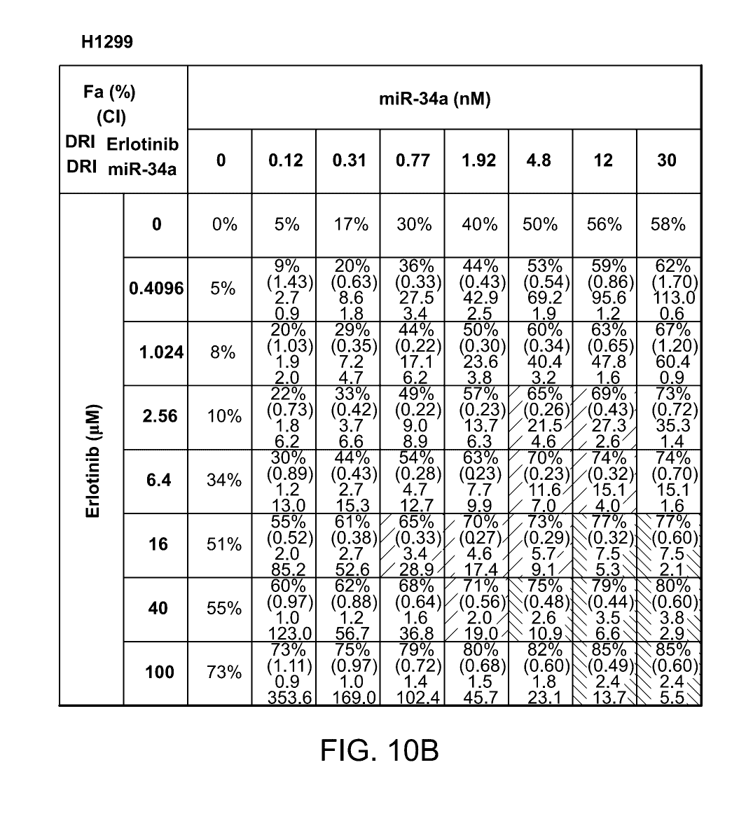

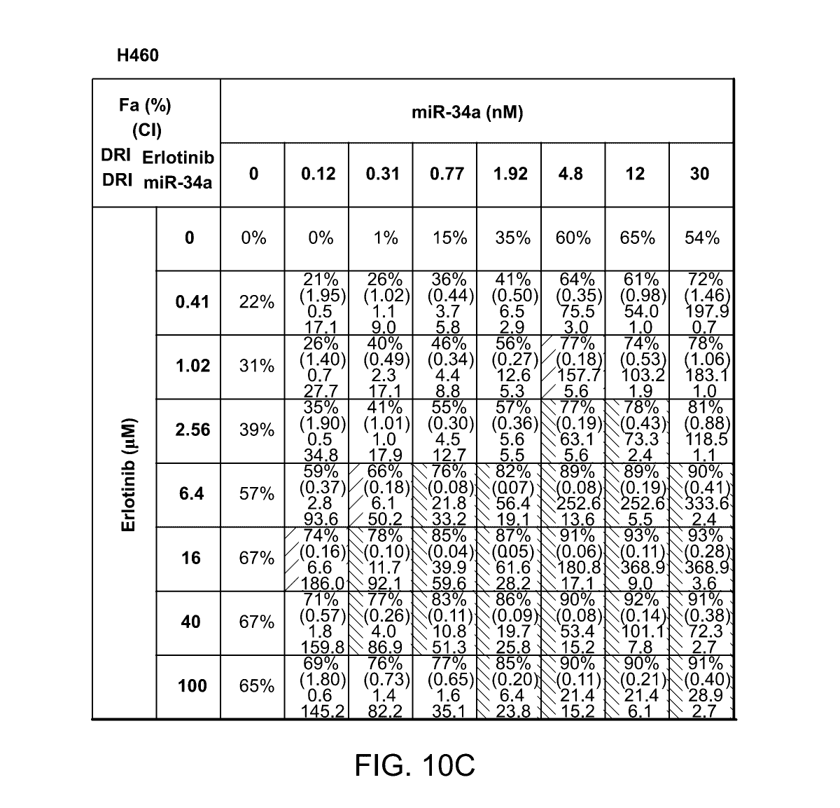

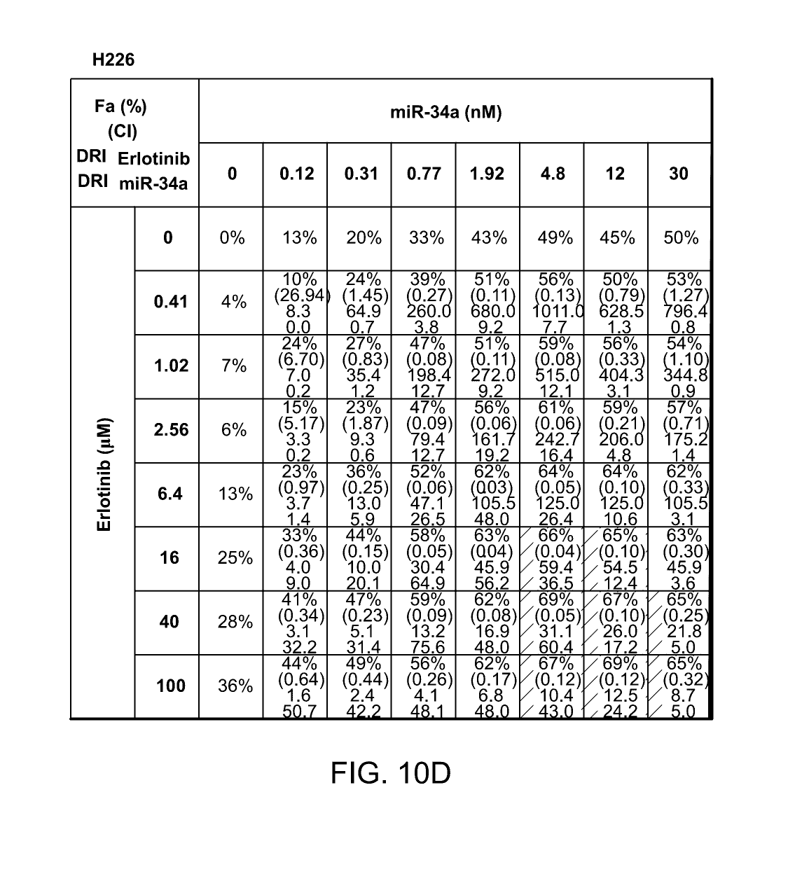

The full range of 49 combinations was also tested in H1299, H460 and H226 cells and confirmed the results obtained with A549 cells (FIGS. 10A-D). Multiple ratios provided good synergy, and the ones with higher potency clustered to the ones with higher drug concentrations. Among these were many that met our cut-offs and produced >75% cancer cell inhibition, CI<0.6, and DRI >2 for each drug.

Erlotinib and miR-34a Cooperate Synergistically in Hepatocellular Carcinoma Cells

To investigate whether the cooperative activity of erlotinib and miR-34a has utility in other cancer indications, we probed this combination in cell models of hepatocellular carcinoma. Liver cancer was chosen as test platform because erlotinib is moderately effective in patients with advanced liver as a single agent and failed to prolong overall survival and time-to-progression in combination with sorafenib (Philip P A, Mahoney M R, Allmer C, Thomas J, Pitot H C, et al. (2005) Phase II study of Erlotinib (OSI-774) in patients with advanced hepatocellular cancer. J Clin Oncol 23: 6657-63. Thomas M B, Chadha R, Glover K, Wang X, Morris J, et al. (2007) Phase 2 study of erlotinib in patients with unresectable hepatocellular carcinoma. Cancer 110: 1059-67. Zhu A X, Rosmorduc O, Evans J, Ross P, Santoro A, et al. (2012) SEARCH: A phase III, randomized, double-blind, placebo-controlled trial of sorafenib plus erlotinib in patients with hepatocellular carcinoma (HCC). 37th Annual European Society for Medical Oncology Congress, Vienna, Austria, September 28-October 2 (abstr 917)).

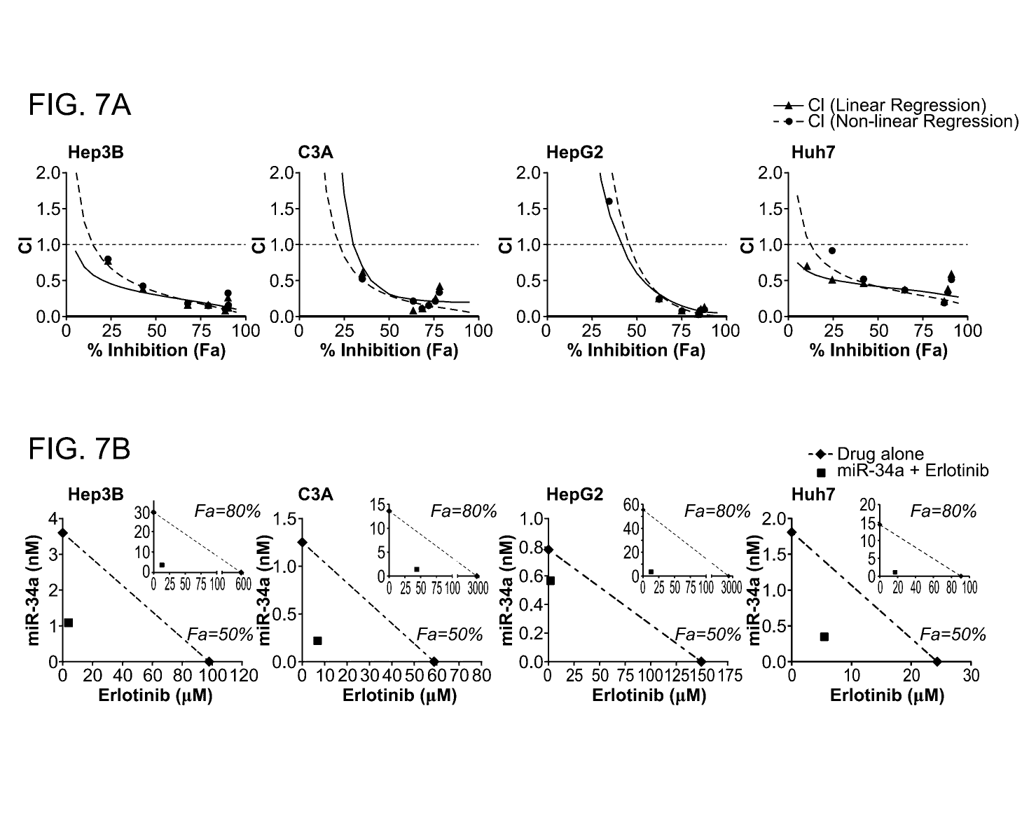

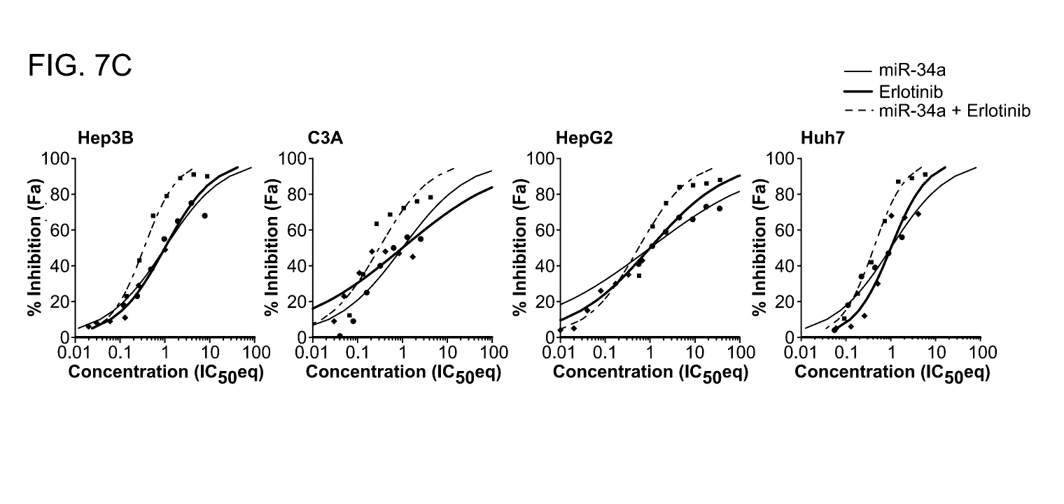

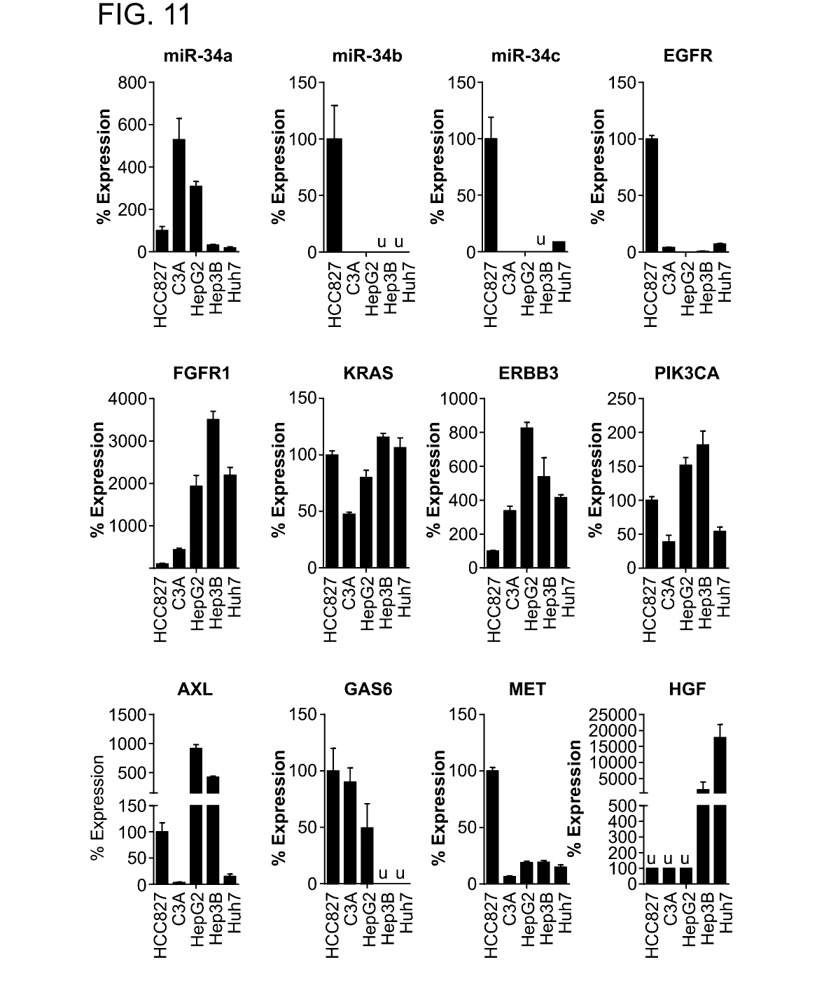

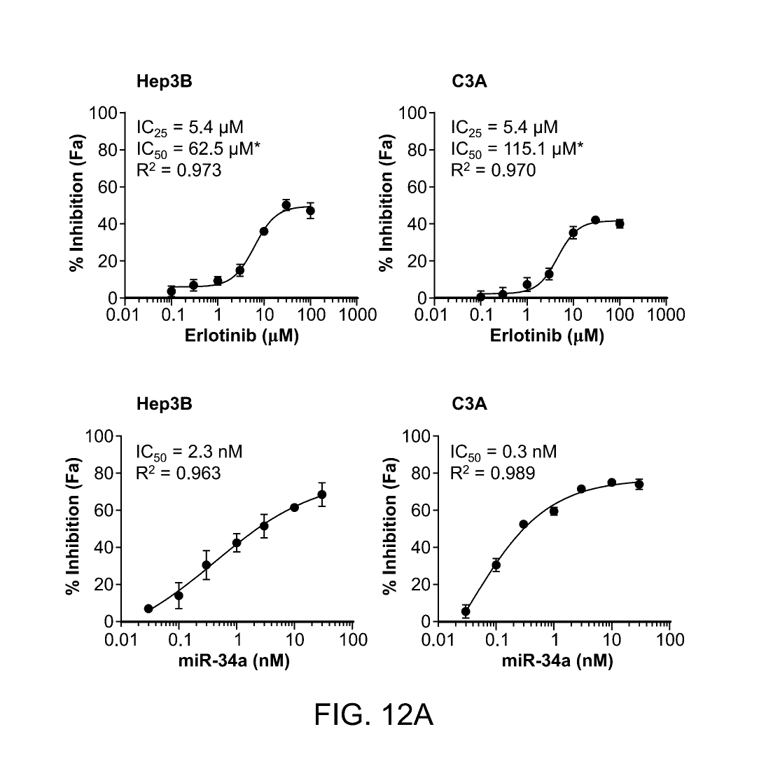

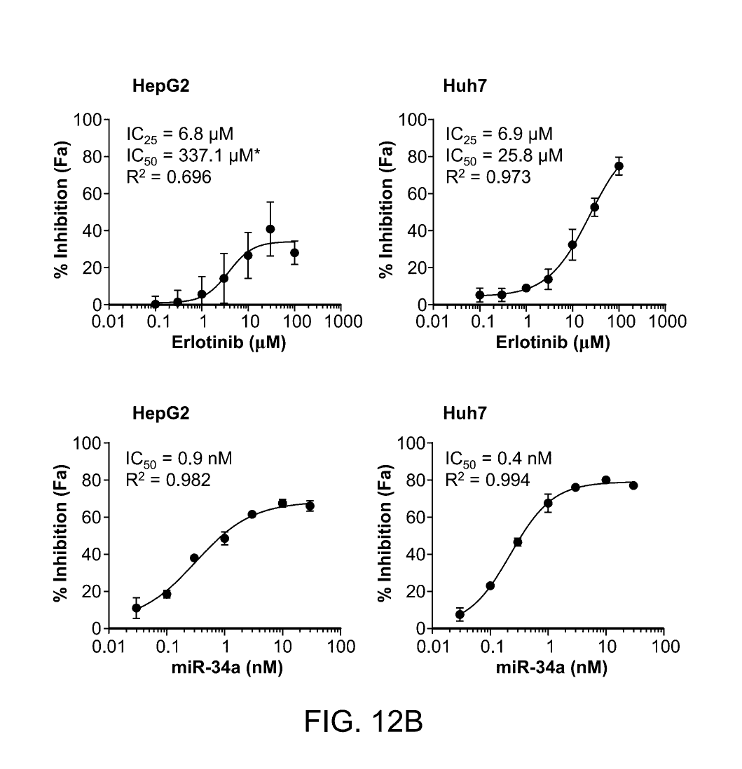

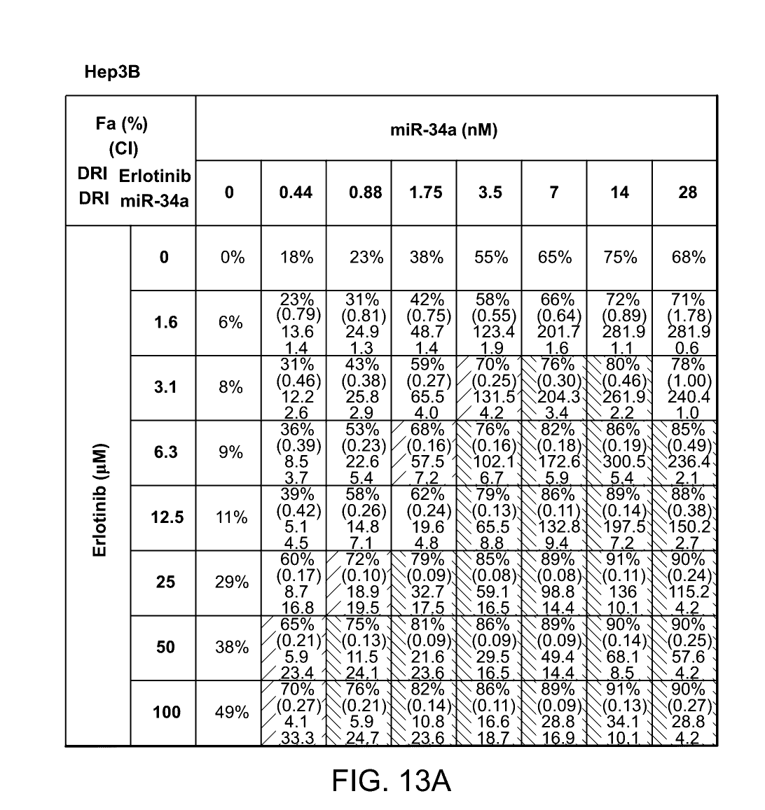

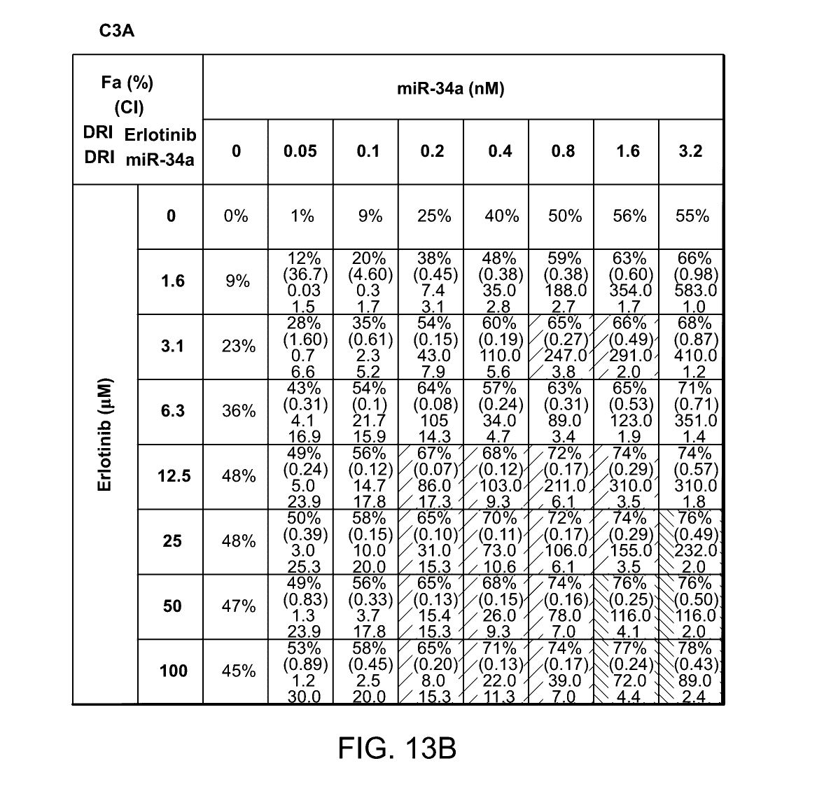

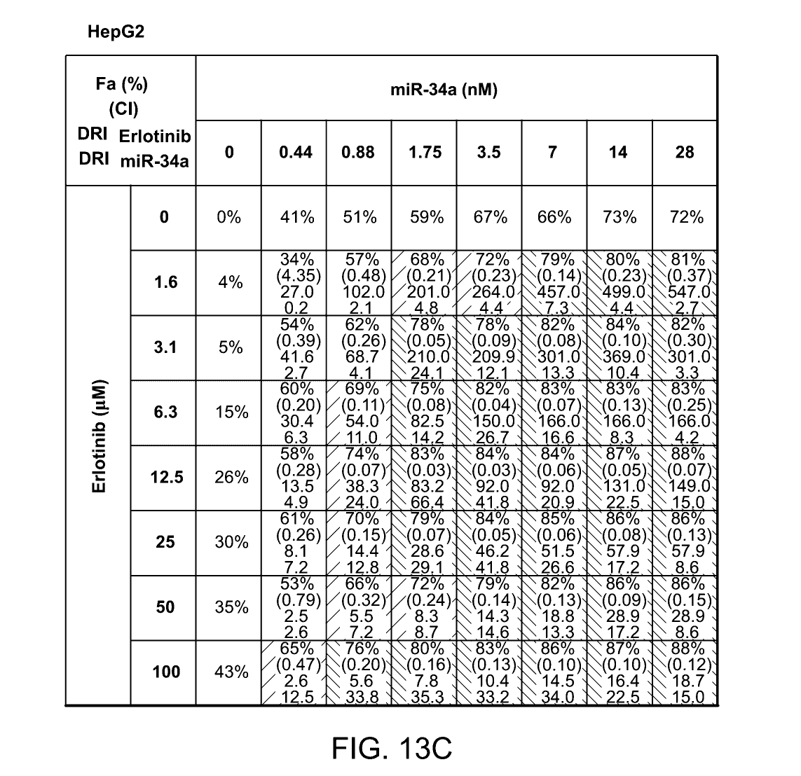

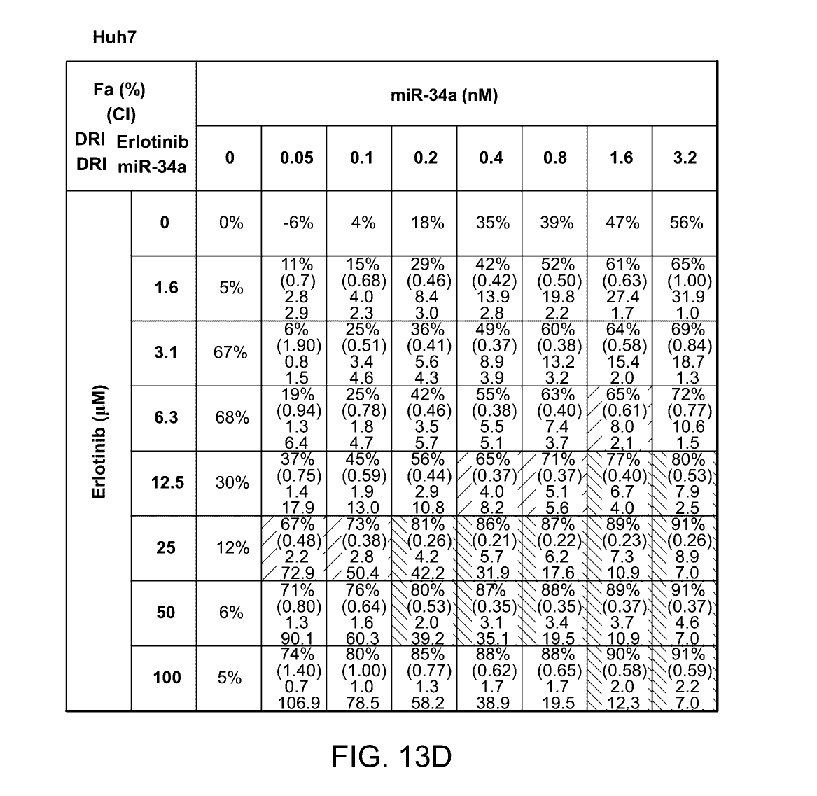

In addition, MRX34, a miR-34a liposome currently in clinical testing, effectively eliminated liver tumors in preclinical animal studies and therefore may be an attractive agent in combination with erlotinib. Cell models used included Hep3B, C3A, HepG2 and Huh7, several of which showed an upregulation of erlotinib-resistance genes, AXL, HGF, FGFR1 and ERBB3 in comparison to an erlotinib-sensitive lung cancer line (FIG. 11). Collectively, levels of miR-34 family members were low or undetectable in liver cancer cells. In agreement with our expectation, IC₅₀ values of erlotinib were 25 μM or greater in these four cell lines (FIGS. 12A-B). The IC₅₀ values of miR-34a ranged between 0.3 and 2.3 nM and, thus, were similar to those in lung cancer cells. These values were used as a guide to combine erlotinib and miR-34a at a fixed ratio of IC₅₀:IC₅₀ and to produce CI, isoboles and IC₅₀ eq values (FIG. 7). In addition, each combination was also tested in a matrix of different concentrations to assess the combinatorial effects across multiple ratios (FIGS. 13A-D). Our data predict strong synergy between erlotinib and miR-34a in all cell lines tested. Synergy was observed at high levels of cancer cell inhibition and, hence, occurs within the desirable range of activity (FIG. 7A). This result is confirmed by the IC₅₀ eq curve shift analyses indicating synergy at higher dose and effect levels. The analysis also shows that the maximal inhibitory activity of the combination is substantially expanded compared to those of the single agents (FIG. 7C). Isobolograms demonstrate a stark reduction of the erlotinib dose when used with miR-34a to induce 50% inhibition or greater, such as 80% (FIG. 7B). In combination, erlotinib can be used at concentrations as low as 2 μM to inhibit cancer cells by 50%, thereby lowering its dose by 75-fold compared to its single use (see HepG2). Synergy is not limited to a specific ratio but is apparent across most ratios tested (FIGS. 13A-D). Thus, the data are similar to those generated in lung cancer cells and predict enhanced efficacy for the erlotinib-miR-34a combination in cancers where erlotinib alone is insufficient.

Discussion

An accurate evaluation of drug-drug interactions is complex because outcomes depend on drug ratios, drug concentrations and desired potency (Chou T C (2010) Drug combination studies and their synergy quantification using the Chou-Talalay method. Cancer Res 70: 440-6). To investigate the pharmacological relationship between miR-34a mimics and erlotinib, we used multiple analytical approaches to reveal drug enhancements (“Fixed Concentration” model) and to distinguish between additivity, antagonism and synergy (“Fixed Ratio” model). We examined CI values, isobolograms and IC₅₀ equivalents derived from linear or non-linear data regression. Our data show that miR-34a augments the sensitivity to erlotinib in all cancer cells tested—whether they were associated with primary or secondary/acquired resistance. A plausible explanation is provided by the fact that tumor suppressor miRNAs inhibit numerous cancer pathways. In support of this hypothesis, AXL and MET, gene products specifically linked to erlotinib resistance, are directly repressed by miR-34a (Kaller M, Liffers S T, Oeljeklaus S, Kuhlmann K, Roh S, et al. (2011) Genome-wide characterization of miR-34a induced changes in protein and mRNA expression by a combined pulsed SILAC and microarray analysis. Mol Cell Proteomics 10: M111 010462. Mudduluru G, Ceppi P, Kumarswamy R, Scagliotti G V, Papotti M, et al. (2011) Regulation of Axl receptor tyrosine kinase expression by miR-34a and miR-199a/b in solid cancer. Oncogene 30: 2888-99. He L, He X, Lim L P, de Stanchina E, Xuan Z, et al. (2007) A microRNA component of the p53 tumour suppressor network. Nature 447: 1130-4.).

Unexpectedly, erlotinib also enhanced the therapeutic effects of the miR-34a mimic, despite existing evidence implicating miR-34a in the control of multiple oncogenic signaling pathways, including the EGFR pathway (Lal A, Thomas M P, Altschuler G, Navarro F, O'Day E, et al. (2011) Capture of microRNA-bound mRNAs identifies the tumor suppressor miR-34a as a regulator of growth factor signaling. PLoS Genet 7: e1002363.). Thus, this result demonstrates that a miRNA mimic can synergize with a single gene-directed therapy and invites the search for other combinations. Accordingly, in various additional embodiments, the present invention includes combinations of miR-34a with other EGFR inhibitors, such as gefitinib, afatinib, panitumumab and cetuximab, as well as HER2 inhibitors such as lapatinib, pertuzumab and trastuzumab.

In lung cancer cells with acquired resistance (HCC827^{res}), adding a small amount of miR-34a was capable of reducing erlotinib IC₅₀ values below 0.1 μM. This is a remarkable result and suggests that miR-34a can render this cell line equally erlotinib-sensitive compared to parental HCC827 cells. In lung cancer cells with primary resistance, the IC₅₀ dose requirement for erlotinib decreased by 4- to 46-fold and was approximately 2 μM. This may be within the range of concentrations that have clinical utility (Sharma S V, Bell D W, Settleman J Haber D A (2007) Epidermal growth factor receptor mutations in lung cancer. Nat Rev Cancer 7: 169-81.). Erlotinib is given as a daily, oral dose of up to 150 mg. Although the clinical dose level of MRX34 has yet to be established, the molar ratios between miR-34a and erlotinib used in the clinic are likely within the range of ratios that have shown synergy in our cell studies.

Erlotinib is currently used as a first-line therapy for NSCLC patients with activating EGFR mutations. It is also used as a maintenance therapy after chemotherapy and second- and third-line therapy for locally advanced or metastatic NSCLC that has failed at least one prior chemotherapy regimen. Clinical trials failed to demonstrate a survival benefit of erlotinib in combination with cisplatin/gemcitabine or carboplatin/paclitaxel compared to conventional chemotherapies alone (Id. Herbst R S, Prager D, Hermann R, Fehrenbacher L, Johnson B E, et al. (2005) TRIBUTE: a phase III trial of erlotinib hydrochloride (OSI-774) combined with carboplatin and paclitaxel chemotherapy in advanced non-small-cell lung cancer. J Clin Oncol 23: 5892-9.). A recent Phase III trial, investigating erlotinib plus sorafenib in HCC, also did not meet its endpoint (Zhu A X, Rosmorduc O, Evans J, Ross P, Santoro A, et al. (2012) SEARCH: A phase III, randomized, double-blind, placebo-controlled trial of sorafenib plus erlotinib in patients with hepatocellular carcinoma (HCC). 37th Annual European Society for Medical Oncology Congress, Vienna, Austria, September 28-October 2 (abstr 917)). Thus, other approaches for combination therapies are desired. Our data show that the erlotinib plus miR-34a combination is particularly effective and may substantially broaden the NSCLC patient population that can be treated with erlotinib. The combination was similarly synergistic in HCC cells, suggesting that the synergistic interaction is a result of their molecular mechanisms of action and can also be applied to cancers other than NSCLC.

### Example 6

### Lapatinib and miR-34 Mimics (miR-Rx34) Synergize in Breast Cancer Cells

The human breast cancer cell lines BT-549, T47D, MDA-MD-231 and MCF-7 (from ATCC) were used to evaluate the combinatorial effects of mir-Rx34 and lapatinib. Lapatinib was purchased from LC Laboratories (Woburn, Mass.). Synthetic miR-34a and miR-NC mimics were manufactured by Life Technologies (Ambion, Austin, Tex.). To determine the IC₅₀ value of each drug alone, 2,000-3,500 cells per well were seeded in a 96-well plate format and treated with either lapatinib or miR-34a as follows. (i) miR-34a mimics were reverse-transfected in triplicates in a serial dilution (0.03-30 nM) using RNAiMax lipofectamine from Invitrogen according to a published protocol. As controls, cells were also transfected with RNAiMax alone (mock). Cells were incubated with AlamarBlue (Invitrogen) 6 days post transfection to determine cellular proliferation. Proliferation data were normalized to mock-transfected cells. (ii) Lapatinib, prepared as a 10 mM stock solution in dimethyl sulfoxide (DMSO), was added to cells one day after seeding at a final concentration ranging from 0.1 and 100 μM. Solvent alone (1% final DMSO in all cell lines) was added to cells in separate wells as a control. Three days thereafter, cellular proliferation was measured by AlamarBlue and normalized to the solvent control.

The combination studies were carried out at ˜IC₅₀ ratio of lapatinib and miR-Rx34 (ratio=IC₅₀ lapatinib/IC₅₀ miR-Rx34). Cells were treated with lapatinib in combination with miR-Rx34a at a concentration approximately equal to its corresponding IC₅₀ and concentrations within 2 fold increments above or below. The ratios of lapatinib/miR-Rx34a are 4000 in BT-549, 3333.3 in MDA-MD-231, 5000 in MCF-7 and 6000 in T47D. Cells were reversed transfected with miR-Rx34a, lapatinib were added 3 days post transfection, and cell proliferation were measured 3 days post lapatinib addition by AlamarBlue.

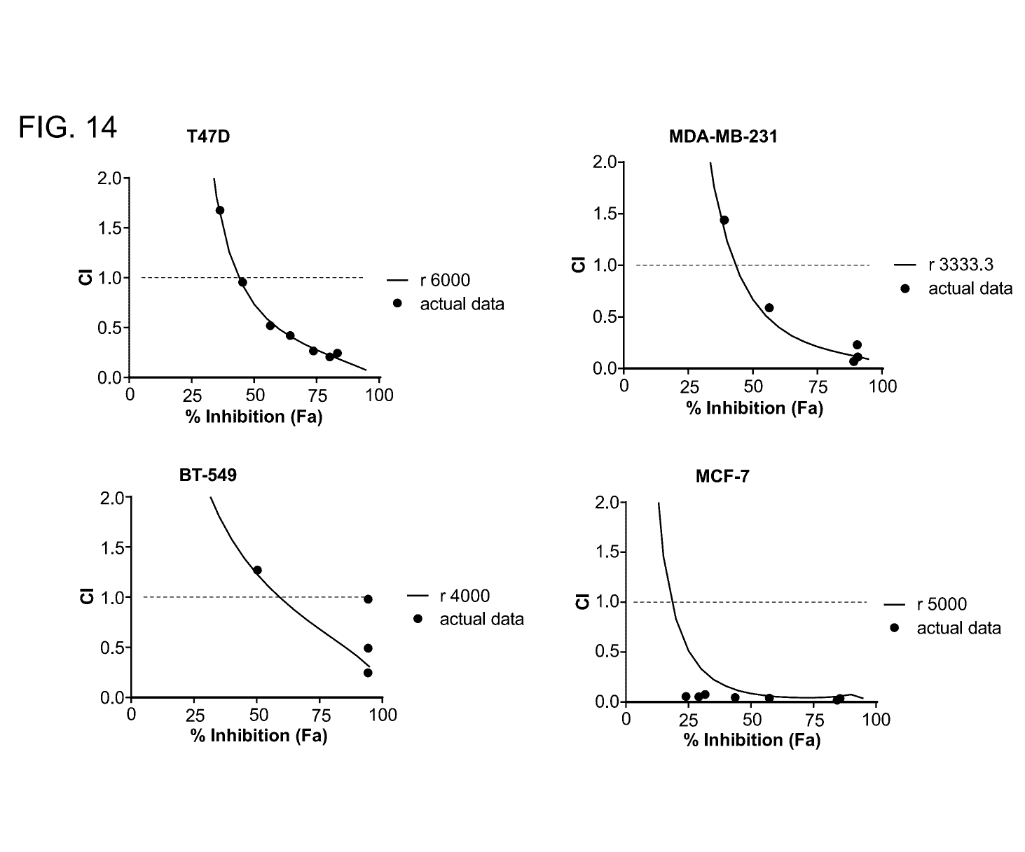

CI values were calculated based on non-linear regression of dose-response curves of the single agents and when used in combination, and are shown relative to the level of cancer cell inhibition on an axis from 0 (no inhibition) to 1(100% inhibition). Combinations that are considered synergistic and have clinical value are those with a low CI value (<0.6) at maximal cancer cell inhibition. As shown in FIG. 14, miR-Rx34 synergized with lapatinib across all four breast cancer cell lines (BT-549, MCF-7, MDA-MB-231, T47D). Symbols represent CI values derived from actual data points. CI, combination index; Fa, fraction affected (=inhibition of proliferation); CI=1, additivity; CI>1, antagonism; CI<1, synergy.

### Example 7

### Erlotinib+MRX34 Therapy in NSCLC

To treat patients with non-small cell lung cancer, a MRX34+erlotinib combination can be used as follows. Patient is given a daily oral dose of 150, 100, or 50 mg erlotinib and an intravenous 30 mm to 3 hr infusion of MRX34 at dose levels ranging from 50 mg/m² to 165 mg/m². In particular situations, MRX34 is given at dose levels of 50, 70, 93, 124, or 165 mg/m².

In another example erlotinib is given as a daily oral dose of 150, 100, or 50 mg and MRX34 is given three twice a week (for instance Mondays and Thursdays) during a 30 mm to 3 hr infusion at dose levels ranging from 50 mg/m² to 165 mg/m². In particular situations, MRX34 is given at dose levels of 50, 70, 93, 124 or 165 mg/m².

In another example, erlotinib is given as a daily oral dose of 150, 100, or 50 mg and MRX34 is given daily by an intravenous 30 mm to 3 hr infusion at dose levels ranging from 50 mg/m² to 165 mg/m² on five consecutive days with the following two days off per week. In particular situations, MRX34 is given at dose levels of 50, 70, 93, 124 or 165 mg/m².

### Example 8

### Erlotinib+MRX34 Therapy in Pancreatic Cancer

To treat patients with pancreatic cancer, for example pancreatic ductal adenocarcinoma, a MRX34+erlotinib combination can be used as follows. Patient is given a daily oral dose of 100 or 50 mg erlotinib and an intravenous 30 mm to 3 hr infusion of MRX34 at dose levels ranging from 50 mg/m² to 165 mg/m². In particular situations, MRX34 is given at dose levels of 50, 70, 93, 124 or 165 mg/m².

In another example erlotinib is given as a daily oral dose of 100 or 50 mg, and MRX34 is given three twice a week (for instance Mondays and Thursdays) during a 30 mm to 3 hr infusion at dose levels ranging from 50 mg/m² to 165 mg/m². In particular situations, MRX34 is given at dose levels of 50, 70, 93, 124 or 165 mg/m².

In another example, erlotinib is given as a daily oral dose of 100 or 50 mg, and MRX34 is given daily by an intravenous 30 mm to 3 hr infusion at dose levels ranging from 50 mg/m² to 165 mg/m² on five consecutive days with the following two days off per week. In particular situations, MRX34 is given at dose levels of 50, 70, 93, 124 or 165 mg/m².

### Example 9

### Lapatinib+MRX34 Therapy in Breast Cancer

To treat patients with breast cancer, for example hormone receptor-positive, HER2-positive metastatic breast cancer, a MRX34+lapatinib combination can be used as follows. Patient is given a daily oral dose of 1500, 1250, 1000, or 750 mg lapatinib and an intravenous 30 mm to 3 hr infusion of MRX34 at dose levels ranging from 50 mg/m² to 165 mg/m². In particular situations, MRX34 is given at dose levels of 50, 70, 93, 124 or 165 mg/m².

In another example lapatinib is given as a daily oral dose of 1500, 1250, 1000, or 750 mg, and MRX34 is given three twice a week (for instance Mondays and Thursdays) during a 30 mm to 3 hr infusion at dose levels ranging from 50 mg/m² to 165 mg/m². In particular situations, MRX34 is given at dose levels of 50, 70, 93, 124 or 165 mg/m².

In another example, lapatinib is given as a daily oral dose of 1500, 1250, 1000, or 750 mg, and MRX34 is given daily by an intravenous 30 mm to 3 hr infusion at dose levels ranging from 50 mg/m² to 165 mg/m² on five consecutive days with the following two days off per week. In particular situations, MRX34 is given at dose levels of 50, 70, 93, 124 or 165 mg/m².

In another example, lapatinib and MRX34 is given as described above and combined with capecitabine 2,000 mg/m²/day (administered orally in 2 doses approximately 12 hours apart) on Days 1-14 in a repeating 21-day cycle.

In another example, lapatinib and MRX34 are given as described above and combined with letrozole 2.5 mg once daily

### Example 10

### Afatinib+MRX34 Therapy in NSCLC

To treat patients with non-small cell lung cancer, a MRX34+afatinib combination can be used as follows. Patient is given a daily oral dose of 40, 30, or 20 mg afatinib and an intravenous 30 mm to 3 hr infusion of MRX34 at dose levels ranging from 50 mg/m² to 165 mg/m². In particular situations, MRX34 is given at dose levels of 50, 70, 93, 124 or 165 mg/m².

In another example afatinib is given as a daily oral dose of 40, 30, or 20 mg, and MRX34 is given three twice a week (for instance Mondays and Thursdays) during a 30 mm to 3 hr infusion at dose levels ranging from 50 mg/m² to 165 mg/m². In particular situations, MRX34 is given at dose levels of 50, 70, 93, 124 or 165 mg/m².

In another example, afatinib is given as a daily oral dose of 40, 30, or 20 mg, and MRX34 is given daily by an intravenous 30 mm to 3 hr infusion at dose levels ranging from 50 mg/m² to 165 mg/m² on five consecutive days with the following two days off per week. In particular situations, MRX34 is given at dose levels of 50, 70, 93, 124 or 165 mg/m².

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. Those skilled in the art will recognize, or be able to ascertain using no more than routine experimentation, many equivalents to the specific embodiments of the invention described herein. Such equivalents are intended to be encompassed by the following claims.

APPENDIX A

| | MicroRNA_ | | | | | | | | | |
|---|---|---|---|---|---|---|---|---|---|---|
| | Accession | SEQ | | HCC827-pool- | HCC827- | HCC827- | HCC827- | | | |
| MicroRNA | ID | ID NO: | MicroRNA_Seq | Erlot-res-1 | Erlot-res-5 | Erlot-res-6 | Erlot-res-7 | Calu-3 | H460 | H1299 |
| hsa- | MIMAT0005865 | 8 | GUGCCAGCUGCA | -2.3078918 | -1.448326776 | -1.293321444 | -0.562809925 | -2.299436598 | -1.456902705 | -1.573259772 |
| miR-1202 | | | GUGGGGGAG | | | | | | | |
| hsa- | MIMAT0000078 | 9 | AUCACAUUGCCA | -2.039430924 | -1.831164437 | -1.403271144 | -0.956618884 | 0.438912408 | 0.565118162 | 1.571696324 |
| miR-23a | | | GGGAUUUCC | | | | | | | |
| hsa- | MIMAT0000068 | 10 | UAGCAGCACAUA | -1.911713116 | -1.360645527 | -0.904863281 | -0.454846203 | -0.409429571 | -0.435988256 | 0.024669316 |
| miR-15a | | | AUGGUUUGUG | | | | | | | |
| hsa- | MIMAT0000067 | 11 | UGAGGUAGUAGA | -1.892652144 | -1.026085283 | -0.6598075 | -0.401923403 | -0.286856206 | -0.541935862 | -1.89899012 |
| let-7f | | | UUGUAUAGUU | | | | | | | |
| hsa- | MIMAT0000077 | 12 | AAGCUGCCAGUU | -1.847409898 | -0.894406144 | -0.626883029 | -0.097520593 | -0.807783769 | 0.242592756 | 0.322082045 |
| miR-22 | | | GAAGAACUGU | | | | | | | |
| hsa- | MIMAT0004504 | 13 | UGCUAUGCCAAC | 1.825783686 | -1.137373695 | -0.844703668 | -0.442944586 | -0.683205721 | -1.635546721 | -0.9650992 |
| miR-31* | | | AUAUUGCCAU | | | | | | | |
| hsa- | MIMAT0000080 | 14 | UGGCUCAGUUCA | -1.823840323 | -1.147440145 | -0.856021603 | -0.5095624 | 1.057604445 | 0.390458206 | 1.643496 |
| miR-24 | | | GCAGGAACAG | | | | | | | |
| hsa- | MIMAT0000084 | 15 | UUCACAGUGGCU | -1.815109494 | -1.011730711 | -0.729862661 | -0.45985578 | -0.076462895 | -0.102329398 | 1.561600646 |
| miR-27a | | | AAGUUCCGC | | | | | | | |
| hsa- | MIMAT0000419 | 16 | UUCACAGUGGCU | -1.763513653 | -0.809562121 | -0.547668429 | -0.325382181 | 0.270655791 | -0.499797932 | -1.649349682 |
| miR-27b | | | AAGUUCUGC | | | | | | | |
| hsa- | MIMAT0000062 | 17 | UGAGGUAGUAGG | -1.747909975 | -1.079998611 | -0.757064122 | -0.376902128 | -0.035846772 | -0.62887642 | -0.764099407 |
| let-7a | | | UUGUAUAGUU | | | | | | | |
| hsa- | MIMAT0000063 | 18 | UGAGGUAGUAGG | -1.7091455 | -1.333477641 | -1.098620618 | -0.692907814 | 1.845648857 | 0.384463611 | -3.397858631 |
| let-7b | | | UUGUGUGGUU | | | | | | | |
| hsa-miR- | MIMAT0005572 | 19 | GUGGGUACGGCC | -1.69129606 | -0.295514709 | -0.273896969 | 0.283666663 | -1.260954557 | -1.101452735 | -1.061210107 |
| 1225-5p | | | CAGUGGGGGG | | | | | | | |
| hsa- | MIMAT0003308 | 20 | AGGGAUCGCGGG | -1.669398488 | -0.624093837 | -0.498046007 | 0.152020025 | -1.696591387 | -1.005822281 | -1.386065395 |
| miR-638 | | | CGGGUGGCGGCC | | | | | | | |
| | | | U | | | | | | | |
| hsa- | MIMAT0000418 | 21 | AUCACAUUGCCA | -1.63857962 | -0.930638445 | -0.66905782 | -0.412164602 | 1.507754029 | 0.139666545 | -1.07339983 |
| miR-23b | | | GGGAUUACC | | | | | | | |
| hsa- | MIMAT0000415 | 22 | UGAGGUAGUAGU | -1.61552766 | -0.803753781 | -0.550016011 | -0.148726238 | 0.946749482 | 2.938321209 | -4.09100538 |
| let-7i | | | UUGUGCUGUU | | | | | | | |
| hsa- | MIMAT0000065 | 23 | AGAGGUAGUAGG | -1.603789762 | -0.970086231 | -0.754099232 | -0.358960851 | -0.201057394 | -0.282990437 | -4.393149655 |
| let-7d | | | UUGCAUAGUU | | | | | | | |
| hsa- | MIMAT0000066 | 24 | UGAGGUAGGAGG | -1.546404672 | -0.688378019 | -0.450562432 | -0.160955378 | 0.832329344 | -0.389780255 | -0.507812338 |
| let-7e | | | UUGUAUAGUU | | | | | | | |
| hsa- | MIMAT0000069 | 25 | UAGCAGCACGUA | -1.541633691 | -0.962311619 | -0.672553038 | -0.18924741 | 0.444538426 | -0.507608102 | 0.307222044 |
| miR-16 | | | AAUAUUGGCG | | | | | | | |
| hsa- | MIMAT0000089 | 26 | AGGCAAGAUGCU | -1.53316035 | -0.873664446 | -0.581789742 | -0.148248845 | -0.018605902 | -1.953495827 | -1.141872046 |
| miR-31 | | | GGCAUAGCU | | | | | | | |
| hsa- | MIMAT0005947 | 27 | GCAUGGGUGGUU | -1.530879826 | -0.841082967 | -0.51613076 | 0.026660344 | -2.653667496 | -0.191709518 | -0.516838192 |
| miR-1308 | | | CAGUGG | | | | | | | |
| hsa- | MIMAT0004494 | 28 | CAACACCAGUCG | -1.500115257 | -0.816522083 | -0.66369233 | -0.239636688 | -1.270422936 | -0.880528079 | 0.278445774 |
| miR-21* | | | AUGGGCUGU | | | | | | | |
| hsa- | MIMAT0000075 | 29 | UAAAGUGCUUAU | -1.443416853 | -0.910668462 | -0.633043509 | -0.117681796 | -1.051128859 | 0.438024175 | 0.293310517 |
| miR-20a | | | AGUGCAGGUAG | | | | | | | |
| hsa- | MIMAT0005893 | 30 | UUUUCAACUCUA | -1.441378663 | -1.196400721 | -1.233762894 | -0.925793154 | -0.728243268 | -0.744531489 | -0.642136452 |
| miR-1305 | | | AUGGGAGAGA | | | | | | | |
| hsa- | MIMAT0005911 | 31 | AUCCCACCUCUG | -1.439005279 | -1.006269975 | -0.679248416 | -0.192224794 | -0.850681086 | -0.021960149 | 0.184444897 |
| miR-1260 | | | CCACCA | | | | | | | |
| hsa-miR- | MIMAT0000760 | 32 | GCCCCUGGGCCU | -1.41884288 | -0.845815588 | -0.660389648 | -0.094429907 | 1.818888569 | 0.01801632 | 0.82040125 |
| 331-3p | | | AUCCUAGAA | | | | | | | |
| hsa- | MIMAT0000076 | 33 | UAGCUUAUCAGA | -1.415035803 | -0.721537612 | -0.41506296 | -0.074803789 | -1.35015672 | -2.333786878 | -0.532466771 |
| miR-21 | | | CUGAUGUUGA | | | | | | | |
| hsa-miR- | MIMAT0004906 | 34 | CGCGGGUGCUUA | -1.412844461 | -0.99671682 | -0.730438902 | -0.103788696 | -0.329147578 | -0.397578879 | -7.304829275 |
| 886-3p | | | CUGACCCUU | | | | | | | |
| hsa- | MIMAT0000420 | 35 | UGUAAACAUCCU | -1.390948986 | -0.697159541 | -0.400661503 | -0.09597978 | -0.275005272 | -2.289180937 | -2.170739733 |
| miR-30b | | | ACACUCAGCU | | | | | | | |
| hsa- | MIMAT0000432 | 36 | UAACACUGUCUG | -1.371721469 | -0.603728357 | -0.349018009 | 0.013424518 | -0.694151732 | -5.336268892 | -5.336268892 |
| miR-141 | | | GUAAAGAUGG | | | | | | | |
| hsa- | MIMAT0000318 | 37 | UAAUACUGCCUG | -1.368296967 | -0.808933158 | -0.543146101 | -0.164304745 | 1.856990486 | -2.279702539 | -2.279702539 |
| miR-200b | | | GUAAUGAUGA | | | | | | | |
| hsa- | MIMAT0000417 | 38 | UAGCAGCACAUC | -1.35381126 | -0.841207127 | -0.67952265 | -0.261184985 | 0.665821102 | -0.85162815 | 0.317574841 |
| miR-15b | | | AUGGUUUACA | | | | | | | |
| hsa-miR- | MIMAT0000085 | 39 | AAGGAGCUCACA | -1.34879208 | -0.401272464 | -0.151758016 | 0.089256127 | -0.561637292 | -2.079199752 | -1.950019657 |
| 28-5p | | | GUCUAUUGAG | | | | | | | |
| hsa- | MIMAT0005942 | 40 | UGGACUGCCCUG | -1.348575636 | -1.520695063 | -1.37621968 | -0.944187792 | -1.095304034 | -0.92449505 | -0.690179589 |
| miR-1288 | | | AUCUGGAGA | | | | | | | |
| hsa- | MIMAT0000074 | 41 | UGUGCAAAUCCA | -1.348426118 | -0.881684593 | -0.509205988 | -0.020997754 | -1.092376002 | 0.810437992 | 0.428166793 |
| miR-19b | | | UGCAAAACUGA | | | | | | | |
| hsa- | MIMAT0000104 | 42 | AGCAGCAUUGUA | -1.343003307 | -0.741467648 | -0.577707185 | -0.230782215 | 0.80844405 | 0.405856816 | 0.548248932 |
| miR-107 | | | CAGGGCUAUCA | | | | | | | |
| hsa- | MIMAT0000070 | 43 | CAAAGUGCUUAC | -1.324480186 | -0.740597327 | -0.376480853 | 0.113026822 | -0.696160473 | 0.467851263 | 0.612743897 |
| miR-17 | | | AGUGCAGGUAG | | | | | | | |
| hsa- | MIMAT0000414 | 44 | UGAGGUAGUAGU | -1.313418574 | -0.442498536 | -0.217774223 | -0.005724252 | 0.631577329 | -0.071223487 | -3.129421235 |
| let-7g | | | UUGUACAGUU | | | | | | | |
| hsa- | MIMAT0005946 | 45 | UCCCACCGCUGC | -1.307709139 | -0.90249743 | -0.686664061 | -0.15028728 | -1.0809174 | 0.018093206 | 0.178606874 |
| miR-1280 | | | CACCC | | | | | | | |
| hsa-miR- | MIMAT0000762 | 46 | ACUGCCCCAGGU | -1.27765774 | -0.982797516 | -0.764129028 | -0.216009242 | -0.996015775 | -0.248380366 | -0.284921127 |
| 324-3p | | | GCUGCUGG | | | | | | | |
| hsa-miR- | MIMAT0005927 | 47 | GUCCCUGUUCAG | -1.274532076 | -0.872440933 | -0.556156785 | -0.035172524 | -0.790475178 | 0.027011151 | 0.401323325 |
| 1274a | | | GCGCCA | | | | | | | |
| hsa-miR- | MIMAT0004697 | 48 | UCGAGGAGCUCA | -1.273401753 | -0.49041455 | -0.202473479 | 0.185675199 | 1.497103671 | -0.267800202 | 0.883207222 |
| 151-5p | | | CAGUCUAGU | | | | | | | |
| hsa- | MIMAT0000510 | 49 | AAAAGCUGGGUU | -1.267779248 | -0.743934416 | -0.640782439 | -0.277685309 | -0.481651521 | -0.220044718 | 0.133376074 |
| miR-320a | | | GAGAGGGCGA | | | | | | | |
| hsa- | MIMAT0005954 | 50 | UCUCGCUGGGGC | -1.261051919 | -0.752174562 | -0.440234007 | -0.070334947 | -0.791310286 | -0.25902338 | -0.087791584 |
| miR-720 | | | CUCCA | | | | | | | |
| hsa- | MIMAT0000281 | 51 | CAAGUCACUAGU | -1.251753225 | -0.448085402 | -0.228640888 | 0.054223935 | -1.401712171 | -1.439758142 | -1.439758142 |
| miR-224 | | | GGUUCCGUU | | | | | | | |
| hsa- | MIMAT0000064 | 52 | UGAGGUAGUAGG | -1.231353437 | -1.075319365 | -0.688284301 | -0.506942137 | 1.547056571 | 1.703175104 | -1.231353437 |
| let-7c | | | UUGUAUGGUU | | | | | | | |
| hsa- | MIMAT0000073 | 53 | UGUGCAAAUCUA | -1.226142699 | -0.993017928 | -0.601477123 | -0.059607456 | -1.226142699 | 1.018799378 | 0.568257276 |
| miR-19a | | | UGCAAAACUGA | | | | | | | |
| hsa-miR- | MIMAT0007890 | 54 | GGAGGGGUCCCG | -1.216785328 | -1.419153449 | -1.237503775 | -0.977338357 | -1.17459539 | -0.854694309 | -0.755931631 |
| 1914* | | | CACUGGGAGG | | | | | | | |
| hsa-miR- | MIMAT0005938 | 55 | UCCCUGUUCGGG | -1.211935759 | -0.850653301 | -0.589724337 | -0.171243904 | -0.704893014 | -0.04878529 | -0.137526341 |
| 1274b | | | CGCCA | | | | | | | |
| hsa- | MIMAT0005792 | 56 | AAAAGCUGGGUU | -1.21119071 | -0.650938496 | -0.511434642 | -0.069412617 | -0.017362855 | 0.126658609 | 0.173713044 |
| miR-320b | | | GAGAGGGCAA | | | | | | | |
| hsa- | MIMAT0005922 | 57 | CGGGCGUGGUGG | -1.168110907 | -0.538042017 | -0.319362073 | -0.007655811 | -0.466072681 | -0.216701772 | -0.140673317 |
| miR-1268 | | | UGGGGG | | | | | | | |
| hsa- | MIMAT0000101 | 58 | AGCAGCAUUGUA | -1.150032238 | -0.595968111 | -0.381666597 | -0.026479078 | 0.880980148 | 0.015438183 | 1.130591479 |
| miR-103 | | | CAGGGCUAUGA | | | | | | | |
| hsa- | MIMAT0002819 | 59 | AACUGGCCCUCA | -1.13878098 | -0.519841726 | -0.343778451 | 0.100900855 | -0.045463248 | 1.969578317 | -1.13878098 |
| miR-193b | | | AAGUCCCGCU | | | | | | | |
| hsa- | MIMAT0003240 | 60 | GAGCCAGUUGGA | -1.123346806 | -0.626539429 | -0.72403975 | -0.407403831 | -0.827504254 | -0.506520216 | -0.326364491 |
| miR-575 | | | CAGGAGC | | | | | | | |
| hsa- | MIMAT0000617 | 61 | UAAUACUGCCGG | -1.119251697 | -0.48470742 | -0.239805322 | 0.165145702 | -0.057982286 | -4.197688718 | -4.197688718 |
| miR-200c | | | GUAAUGAUGGA | | | | | | | |
| hsa- | MIMAT0000245 | 62 | UGUAAACAUCCC | -1.118240544 | -0.393624353 | -0.181668473 | 0.055390091 | 0.014737189 | -2.364088348 | -1.842666627 |
| miR-30d | | | CGACUGGAAG | | | | | | | |
| hsa- | MIMAT0000691 | 63 | CAGUGCAAUGAU | -1.105351904 | -0.614065451 | -0.370857264 | -0.001136732 | 1.553593034 | 0.359525304 | 1.638653224 |
| miR-130b | | | GAAAGGGCAU | | | | | | | |
| hsa- | MIMAT0003326 | 64 | AGGCGGGGCGCC | -1.101968317 | -0.190726002 | -0.067352293 | 0.673522496 | -0.601894414 | -1.023458593 | -1.099989632 |
| miR-663 | | | GCGGGACCGC | | | | | | | |
| hsa- | MIMAT0002874 | 65 | UAGCAGCGGGAA | -1.085614755 | -1.085614755 | -1.085614755 | -1.085614755 | -1.085614755 | -1.085614755 | -1.085614755 |
| miR-503 | | | CAGUUCUGCAG | | | | | | | |
| hsa- | MIMAT0000100 | 66 | UAGCACCAUUUG | -1.076470051 | -0.310601788 | 0.078946231 | 0.324284413 | -0.178439318 | -0.753575651 | 0.137627946 |
| miR-29b | | | AAAUCAGUGUU | | | | | | | |
| hsa- | MIMAT0000267 | 67 | CUGUGCGUGUGA | -1.044222544 | -0.798031627 | -0.509886891 | -0.084496124 | -0.013946523 | -2.368590558 | -4.18254936 |
| miR-210 | | | CAGCGGCUGA | | | | | | | |
| hsa- | MIMAT0000226 | 68 | UAGGUAGUUUCA | -1.043245306 | -1.043245306 | -1.043245306 | -1.004775983 | -0.62929296 | 0.083735234 | 0.463268008 |
| miR-196a | | | UGUUGUUGGG | | | | | | | |
| hsa- | MIMAT0007892 | 69 | CCCCAGGGCGAC | -1.035126986 | 0.921457936 | 1.063248418 | 1.033470964 | -0.43057514 | -1.121407799 | -0.817146682 |
| miR-1915 | | | GCGGCGGG | | | | | | | |
| hsa- | MIMAT0000086 | 70 | UAGCACCAUCUG | -1.029047502 | -0.369337444 | -0.144878943 | 0.247171003 | -0.487443874 | -1.060418541 | -0.138650036 |
| miR-29a | | | AAAUCGGUUA | | | | | | | |
| hsa- | MIMAT0000092 | 71 | UAUUGCACUUGU | -1.012205609 | -0.60398213 | -0.215781267 | 0.19775283 | -0.918511144 | 0.311056615 | 0.256372775 |
| miR-92a | | | CCCGGCCUGU | | | | | | | |
| hsa- | MIMAT0006764 | 72 | AAAAGCUGGGUU | -1.012153571 | -0.492590113 | -0.311689109 | 0.04388849 | 0.56498992 | 0.447600227 | -0.334503009 |
| miR-320d | | | GAGAGGA | | | | | | | |
| hsa-miR- | MIMAT0000761 | 73 | CGCAUCCCCUAG | -0.999020263 | -0.642372431 | -0.375703164 | -0.033725297 | 0.046181274 | -0.247874107 | 0.881839834 |
| 324-5p | | | GGCAUUGGUGU | | | | | | | |
| hsa- | MIMAT0000269 | 74 | UAACAGUCUCCA | -0.973340474 | -0.973340474 | -0.823164626 | -0.296155139 | -0.777082895 | -0.314601787 | 0.124527476 |
| miR-212 | | | GUCACGGCC | | | | | | | |
| hsa- | MIMAT0000261 | 75 | UAUGGCACUGGU | -0.961265033 | 0.065797086 | 0.182416424 | 0.517566509 | 0.4192398 | -0.365820517 | -0.41540488 |
| miR-183 | | | AGAAUUCACU | | | | | | | |
| hsa- | MIMAT0000098 | 76 | AACCCGUAGAUC | -0.915895638 | -0.873981798 | -0.58490195 | -0.240461214 | -5.97914641 | -4.144736345 | 1.38143816 |
| miR-100 | | | CGAACUUGUG | | | | | | | |
| hsa- | MIMAT0000256 | 77 | AACAUUCAACGC | -0.912884309 | -0.358751451 | -0.02842813 | 0.212163872 | 2.082187885 | -0.912884309 | -0.912884309 |
| miR-181a | | | UGUCGGUGAGU | | | | | | | |
| hsa- | MIMAT0000646 | 78 | UUAAUGCUAAUC | -0.905586457 | -0.176539899 | 0.150104991 | 0.388939184 | -1.337365576 | -1.337365576 | -1.337365576 |
| miR-155 | | | GUGAUAGGGGU | | | | | | | |
| hsa- | MIMAT0000423 | 79 | UCCCUGAGACCC | -0.903221219 | -0.698403801 | -0.285271411 | 0.048686037 | -2.311123705 | -1.872243683 | 1.372301416 |
| miR-125b | | | UAACUUGUGA | | | | | | | |
| hsa-miR- | MIMAT0000703 | 80 | UUAUCAGAAUCU | -0.896650083 | -0.484017742 | -0.232418684 | 0.170637114 | -0.007062982 | -0.606507057 | 0.626772392 |
| 361-5p | | | CCAGGGGUAC | | | | | | | |
| hsa- | MIMAT0000087 | 81 | UGUAAACAUCCU | -0.884849862 | -0.884849862 | -0.884849862 | -0.673388348 | 3.359142996 | -0.142121464 | 1.927345367 |
| miR-30a | | | CGACUGGAAG | | | | | | | |
| hsa- | MIMAT0000681 | 82 | UAGCACCAUUUG | -0.872315542 | -0.109227958 | 0.23595823 | 0.589985411 | 0.743487572 | 0.862612577 | -0.92294228 |
| miR-29c | | | AAAUCGGUUA | | | | | | | |
| hsa- | MIMAT0000095 | 83 | UUUGGCACUAGC | -0.868666222 | 0.088946868 | 0.285622048 | 0.639215688 | -0.22243213 | -0.30623436 | -0.30305068 |
| miR-96 | | | ACAUUUUUGCU | | | | | | | |
| hsa- | MIMAT0000255 | 84 | UGGCAGUGUCUU | -0.868221863 | -0.151028131 | 0.108647722 | 0.346907237 | 0.448432018 | 0.208033284 | -2.943926998 |
| miR-34a | | | AGCUGGUUGU | | | | | | | |
| hsa-miR- | MIMAT0002872 | 85 | AAUCCUUUGUCC | -0.833986239 | -1.026161539 | -1.026161539 | -1.026161539 | -1.026161539 | -1.026161539 | -1.026161539 |
| 501-5p | | | CUGGGUGAGA | | | | | | | |
| hsa- | MIMAT0004918 | 86 | CACUGGCUCCUU | -0.830643501 | -1.724167177 | -2.219066115 | -2.052080865 | -1.620499939 | -1.728090091 | -1.766257824 |
| miR-892b | | | UCUGGGUAGA | | | | | | | |
| hsa-miR- | MIMAT0000443 | 87 | UCCCUGAGACCC | -0.7809718 | -0.302410624 | 0.033391369 | 0.24873573 | 0.939439554 | 0.141353358 | 0.29802796 |
| 125a-5p | | | UUUAACCUGUGA | | | | | | | |
| hsa- | MIMAT0002816 | 88 | UGAAACAUACAC | -0.768612234 | -0.287954701 | -0.291899825 | 0.478740067 | -0.626020299 | -0.12857028 | 0.221102667 |
| miR-494 | | | GGGAAACCUC | | | | | | | |
| hsa- | MIMAT0004773 | 89 | UAAUCCUUGCUA | -0.766989114 | -0.766989114 | -0.766989114 | -0.766989114 | -0.766989114 | -0.766989114 | -0.702787099 |
| miR-500 | | | CCUGGGUGAGA | | | | | | | |
| hsa- | MIMAT0000227 | 90 | UUCACCACCUUC | -0.763164498 | -0.3851214 | -0.149215758 | 0.067254138 | -0.244017965 | -1.307386294 | -0.40657683 |
| miR-197 | | | UCCACCCAGC | | | | | | | |
| hsa- | MIMAT0004982 | 91 | UGGGGAGCUGAG | -0.734132565 | -0.096488521 | 0.03050184 | 0.327472184 | -0.552182337 | -0.493644684 | -0.108206179 |
| miR-939 | | | GCUCUGGGGGUG | | | | | | | |
| hsa- | MIMAT0000257 | 92 | AACAUUCAUUGC | -0.716329214 | -0.716329214 | -0.524969086 | 0.004887264 | 1.892901582 | -0.66769063 | -0.716329214 |
| miR-181b | | | UGUCGGUGGGU | | | | | | | |
| hsa- | MIMAT0004983 | 93 | AAGGCAGGGCCC | -0.662709131 | -0.0829482 | -0.035379168 | 0.251013875 | -0.704511881 | -0.947062126 | -0.458955893 |
| miR-940 | | | CCGCUCCCC | | | | | | | |
| hsa-miR- | MIMAT0000753 | 94 | UCUCACACAGAA | -0.644014354 | -0.433700169 | -0.143995978 | 0.182905175 | -0.003523688 | 0.811983984 | 1.421137051 |
| 342-3p | | | AUCGCACCCGU | | | | | | | |
| hsa- | MIMAT0000278 | 95 | AGCUACAUUGUC | -0.624739682 | -0.624739682 | -0.624739682 | -0.624739682 | 0.607098119 | -0.624739682 | 1.744523266 |
| miR-221 | | | UGCUGGGUUUC | | | | | | | |
| hsa- | MIMAT0005793 | 96 | AAAAGCUGGGUU | -0.59008835 | -0.310841664 | -0.163870725 | 0.202213379 | 0.639023983 | 0.500500122 | -0.226951739 |
| miR-320c | | | GAGAGGGU | | | | | | | |
| hsa-miR- | | 97 | | -0.55700528 | -0.02457959 | -0.0119504 | 0.783525659 | 0.525419863 | 0.219942669 | 0.669879629 |
| 923_v12.0 | | | | | | | | | | |
| hsa-miR- | MIMAT0005458 | 98 | GUGAGGACUCGG | -0.555350708 | -0.555350708 | -0.555350708 | -0.231125435 | -0.555350708 | -0.555350708 | -0.405512523 |
| 1224-5p | | | GAGGUGG | | | | | | | |
| hsa- | MIMAT0000093 | 99 | CAAAGUGCUGUU | -0.527647512 | 0.248482075 | 0.406705391 | 0.824979257 | -0.69507469 | -0.152060339 | 1.266545598 |
| miR-93 | | | CGUGCAGGUAG | | | | | | | |
| hsa- | MIMAT0001341 | 100 | CAGCAGCAAUUC | -0.524340924 | -0.524340924 | -0.524340924 | -0.524340924 | -0.524340924 | -0.524340924 | -0.50469192 |
| miR-424 | | | AUGUUUUGAA | | | | | | | |
| hsa- | MIMAT0000252 | 101 | UGGAAGACUAGU | -0.521195563 | -0.521195563 | -0.282207767 | 0.091336881 | -0.521195563 | -0.521195563 | -0.521195563 |
| miR-7 | | | GAUUUUGUUGU | | | | | | | |
| hsa- | MIMAT0000680 | 102 | UAAAGUGCUGAC | -0.468479681 | 0.471623625 | 0.656705802 | 0.967309753 | 0.052220147 | 0.143604873 | 1.341634848 |
| miR-106b | | | AGUGCAGAU | | | | | | | |
| hsa- | MIMAT0001413 | 103 | CAAAGUGCUCAU | -0.464248065 | -0.464248065 | -0.464248065 | -0.059908172 | 0.294760644 | 0.381792713 | 0.343018861 |
| miR-20b | | | AGUGCAGGUAG | | | | | | | |
| hsa-miR- | MIMAT0004602 | 104 | ACAGGUGAGGUU | -0.422098924 | 0.170599613 | 0.224920086 | 0.067469778 | -0.486037619 | -0.486037619 | -0.486037619 |
| 125a-3p | | | CUUGGGAGCC | | | | | | | |
| hsa-miR- | MIMAT0005871 | 105 | UGGCAGGGAGGC | -0.395394401 | 0.485447319 | 0.581316839 | 0.809788168 | -0.680941414 | -1.146472419 | -1.43022159 |
| 1207-5p | | | UGGGAGGGG | | | | | | | |
| hsa- | MIMAT0000266 | 106 | UCCUUCAUUCCA | -0.369917802 | -0.369917802 | -0.369917802 | -0.369917802 | -0.369917802 | -0.369917802 | -0.369917802 |
| miR-205 | | | CCGGAGUCUG | | | | | | | |
| hsa- | MIMAT0000425 | 107 | CAGUGCAAUGUU | -0.345860835 | -0.345860835 | -0.345860835 | -0.207335285 | -0.345860835 | 1.226573844 | 1.746491559 |
| miR-130a | | | AAAAGGGCAU | | | | | | | |
| hsa- | MIMAT0004955 | 108 | AUAUAAUACAAC | -0.323713739 | -0.323713739 | -0.323713739 | -0.205114959 | -0.323713739 | 0.676979718 | 0.371754728 |
| miR-374b | | | CUGCUAAGUG | | | | | | | |
| hsa- | MIMAT0000081 | 109 | CAUUGCACUUGU | -0.29501106 | 0.635393034 | 0.804585798 | 1.174418253 | -0.419804843 | 0.449929149 | 1.386186806 |
| miR-25 | | | CUCGGUCUGA | | | | | | | |
| hsa- | MIMAT0001080 | 110 | UAGGUAGUUUCC | -0.282915685 | -0.282915685 | -0.282915685 | -0.149320971 | -0.282915685 | 1.987177636 | -0.282915685 |
| miR-196b | | | UGUUGUUGGG | | | | | | | |
| hsa- | MIMAT0000447 | 111 | UGUGACUGGUUG | -0.262658369 | 0.192822119 | 0.343121438 | 0.487670629 | -0.262658369 | -0.262658369 | -0.262658369 |
| miR-134 | | | ACCAGAGGGG | | | | | | | |
| hsa- | MIMAT0000727 | 112 | UUAUAAUACAAC | -0.247338066 | -0.247338066 | -0.247338066 | -0.067844612 | -0.247338066 | 0.926209682 | 0.64266363 |
| miR-374a | | | CUGAUAAGUG | | | | | | | |
| hsa- | MIMAT0000253 | 113 | UACCCUGUAGAU | -0.236177715 | -0.236177715 | -0.236177715 | -0.235802513 | 3.837301167 | 0.913784177 | 3.53150715 |
| miR-10a | | | CCGAAUUUGUG | | | | | | | |
| hsa- | MIMAT0000244 | 114 | UGUAAACAUCCU | -0.235758661 | -0.235758661 | -0.235758661 | -0.195216205 | 1.584718703 | -0.235758661 | 0.553725443 |
| miR-30c | | | ACACUCUCAGC | | | | | | | |
| hsa- | MIMAT0000096 | 115 | UGAGGUAGUAAG | -0.173046364 | -0.173046364 | -0.173046364 | -0.173046364 | 0.306700135 | -0.173046364 | -0.173046364 |
| miR-98 | | | UUGUAUUGUU | | | | | | | |
| hsa- | MIMAT0003393 | 116 | AAUGACACGAUC | -0.143430736 | -0.143430736 | -0.028255092 | 0.089330819 | 1.511455577 | 0.491383321 | 1.356412115 |
| miR-425 | | | ACUCCCGUUGA | | | | | | | |
| hsa-miR- | MIMAT0000459 | 117 | AACUGGCCUACA | -0.129417634 | 0.008150595 | 0.232350235 | 0.699072491 | -0.129417634 | -0.129417634 | 1.227022878 |
| 193a-3p | | | AAGUCCCAGU | | | | | | | |
| hsa- | MIMAT0005589 | 118 | UCGGCCUGACCA | -0.111529644 | -0.111529644 | -0.097105641 | 0.245103117 | -0.111529644 | -0.111529644 | -0.111529644 |
| miR-1234 | | | CCCACCCCAC | | | | | | | |
| hsa- | MIMAT0000072 | 119 | UAAGGUGCAUCU | -0.09916664 | -0.09916664 | -0.09916664 | -0.068016083 | -0.09916664 | 0.646886222 | 0.973632847 |
| miR-18a | | | AGUGCAGAUAG | | | | | | | |
| hsa-miR- | MIMAT0000757 | 120 | CUAGACUGAAGC | -0.098916297 | -0.098916297 | -0.098916297 | 0.04965665 | 0.997635022 | -0.098916297 | 1.477871541 |
| 151-3p | | | UCCUUGAGG | | | | | | | |
| hsa-miR- | MIMAT0000457 | 121 | CAUCCCUUGCAU | -0.026440611 | -0.436754399 | -0.504016979 | 0.018831116 | -0.630560529 | -0.723317288 | -0.400854397 |
| 188-5p | | | GGUGGAGGG | | | | | | | |
| hsa- | MIMAT0003180 | 122 | AAUCGUACAGGG | -0.014790346 | -0.014790346 | -0.014790346 | -0.014790346 | -0.014790346 | -0.014790346 | 0.124836731 |
| miR-487b | | | UCAUCCACUU | | | | | | | |
| hsa- | MIMAT0000097 | 123 | AACCCGUAGAUC | 0 | 0 | 0 | 0 | 0 | 3.466613465 | 0.167971017 |
| miR-99a | | | CGAUCUUGUG | | | | | | | |
| hsa- | MIMAT0000710 | 124 | UAAUGCCCCUAA | 0 | 0 | 0 | 0 | 0 | 0.743985514 | 0 |
| miR-365 | | | AAAUCCUUAU | | | | | | | |
| hsa-miR- | MIMAT0004514 | 125 | GCUGGUUUCAUA | 0 | 0 | 0 | 0 | 0 | 0.439394373 | 0.955391683 |
| 29b-1* | | | UGGUGGUUUAGA | | | | | | | |
| hsa-miR- | MIMAT0004795 | 126 | UGAGUGUGUGUG | 0 | 0 | 0 | 0.350532531 | 0.323676447 | 0.30546655 | 0.29579782 |
| 574-5p | | | UGUGAGUGUGU | | | | | | | |
| hsa-miR- | MIMAT0003258 | 127 | GAGCUUAUUCAU | 0 | 0 | 0 | 0 | 0 | 0.185696564 | 0.261026048 |
| 590-5p | | | AAAAGUGCAG | | | | | | | |
| hsa- | MIMAT0000455 | 128 | UGGAGAGAAAGG | 0 | 0 | 0 | 0 | 0.324354316 | 0.053950984 | 0.710155315 |
| miR-185 | | | CAGUUCCUGA | | | | | | | |
| hsa-miR- | MIMAT0004507 | 129 | AGGUUGGGAUCG | 0 | 0 | 0 | 0 | 0 | 0.036381842 | 0 |
| 92a-1* | | | GUUGCAAUGCU | | | | | | | |
| hsa- | MIMAT0000222 | 130 | CUGACCUAUGAA | 0 | 0 | 0 | 0 | 4.005187799 | 0 | 0 |
| miR-192 | | | UUGACAGCC | | | | | | | |
| hsa- | MIMAT0000460 | 131 | UGUAACAGCAAC | 0 | 0 | 0 | 0 | 1.715109171 | 0 | 0 |
| miR-194 | | | UCCAUGUGGA | | | | | | | |
| hsa- | MIMAT0000682 | 132 | UAACACUGUCUG | 0 | 0 | 0 | 0 | 1.647954727 | 0 | 0 |
| miR-200a | | | GUAACGAUGU | | | | | | | |
| hsa- | MIMAT0000082 | 133 | UUCAAGUAAUCC | 0 | 0 | 0.036373993 | 0.370515843 | 1.449399428 | 0 | 1.037333027 |
| miR-26a | | | AGGAUAGGCU | | | | | | | |
| hsa- | MIMAT0001536 | 134 | UAAUACUGUCUG | 0 | 0 | 0 | 0 | 1.35891655 | 0 | 0 |
| miR-429 | | | GUAAAACCGU | | | | | | | |
| hsa- | MIMAT0000088 | 135 | CUUUCAGUCGGA | 0 | 0 | 0 | 0 | 1.113180526 | 0 | 0.917141587 |
| miR-30a* | | | UGUUUGCAGC | | | | | | | |
| hsa- | MIMAT0000272 | 136 | AUGACCUAUGAA | 0 | 0 | 0 | 0 | 0.963760311 | 0 | 0 |
| miR-215 | | | UUGACAGAC | | | | | | | |
| hsa- | MIMAT0000279 | 137 | AGCUACAUCUGG | 0 | 0 | 0 | 0 | 0.931583904 | 0 | 0 |
| miR-222 | | | CUACUGGGU | | | | | | | |
| hsa- | MIMAT0000689 | 138 | CACCCGUAGAAC | 0 | 0 | 0 | 0 | 0.909684656 | 0 | 0.196735502 |
| miR-99b | | | CGACCUUGCG | | | | | | | |
| hsa-miR- | MIMAT0000705 | 139 | AAUCCUUGGAAC | 0 | 0 | 0 | 0 | 0.256184549 | 0 | 0.78346423 |
| 362-5p | | | CUAGGUGUGAGU | | | | | | | |
| hsa- | MIMAT0000083 | 140 | UUCAAGUAAUUC | 0 | 0 | 0 | 0 | 0.212867288 | 0 | 0 |
| miR-26b | | | AGGAUAGGU | | | | | | | |
| hsa- | MIMAT0000692 | 141 | UGUAAACAUCCU | 0 | 0 | 0 | 0 | 0.197564105 | 0 | 0 |
| miR-30e | | | UGACUGGAAG | | | | | | | |
| hsa- | MIMAT0000758 | 142 | UAUGGCUUUUCA | 0 | 0 | 0 | 0 | 0.057551371 | 0 | 0 |
| miR-135b | | | UUCCUAUGUGA | | | | | | | |
| hsa- | MIMAT0000688 | 143 | CAGUGCAAUAGU | 0 | 0 | 0 | 0 | 0.043505468 | 0 | 0.419594758 |
| miR-301a | | | AUUGUCAAAGC | | | | | | | |
| hsa- | MIMAT0003237 | 144 | GUCCGCUCGGCG | 0 | 1.08124927 | 1.236404081 | 0.86832963 | 0 | 0 | 0 |
| miR-572 | | | GUGGCCCA | | | | | | | |
| hsa- | MIMAT0004911 | 145 | CUGCCCUGGCCC | 0 | 0.75395414 | 0.8104504 | 0.608156372 | 0 | 0 | 0 |
| miR-874 | | | GAGGGACCGA | | | | | | | |
| hsa- | MIMAT0005583 | 146 | UCACACCUGCCU | 0 | 0.178207492 | 0.353500671 | 0.443106593 | 0 | 0 | 0 |
| miR-1228 | | | CGCCCCCC | | | | | | | |
| hsa- | MIMAT0004610 | 147 | CUGGUACAGGCC | 0 | 0.416708808 | 0.468705251 | 0.40955156 | 0 | 0 | 0 |
| miR-150* | | | UGGGGGACAG | | | | | | | |
| hsa-miR- | MIMAT0004687 | 148 | ACUCAAACUGUG | 0 | 0 | 0.088434336 | 0.116087378 | 0 | 0 | 0.28059595 |
| 371-5p | | | GGGGCACU | | | | | | | |
| hsa-miR- | MIMAT0004905 | 149 | CGGGUCGGAGUU | 0 | 0.107643027 | 0.294825976 | 0 | 0 | 0 | 0 |
| 886-5p | | | | | | | | | | |
| hcmv-miR- | MIMAT0001579 | 150 | UGACAAGCCUGA | 0 | 0.28067552 | 0.187139145 | 0 | 0 | 0 | 0 |
| US5-1 | | | CGAGAGCGU | | | | | | | |
| hsa- | MIMAT0000424 | 151 | UCACAGUGAACC | 0 | 0 | 0 | 0 | 0 | 0 | 0.93141386 |
| miR-128 | | | GGUCUCUUU | | | | | | | |
| hsa-miR- | MIMAT0002888 | 152 | CAUGCCUUGAGU | 0 | 0 | 0 | 0 | 0 | 0 | 0.304017974 |
| 532-5p | | | GUAGGACCGU | | | | | | | |
| hsa-miR- | MIMAT0004614 | 153 | UGGGUCUUUGCG | 0 | 0 | 0 | 0 | 0 | 0 | 0.123513165 |
| 193a-5p | | | GGCGAGAUGA | | | | | | | |
| hsa-miR- | MIMAT0003233 | 154 | GCGACCCAUACU | 0 | 0 | 0 | 0 | 0 | 0 | 0.057234091 |
| 551b | | | UGGUUUCAG | | | | | | | |
| hsa- | MIMAT0003338 | 155 | UACCCAUUGCAU | 0 | 0 | 0 | 0 | 0 | 0 | 0.036366375 |
| miR-660 | | | AUCGGAGUUG | | | | | | | |
| hsa- | MIMAT0000449 | 156 | UGAGAACUGAAU | 0.012889515 | 1.522130001 | 1.728363503 | 1.834331129 | 0 | 0 | 0 |
| miR-146a | | | UCCAUGGGUU | | | | | | | |
| hsa- | MIMAT0005826 | 157 | CCGUCGCCGCCA | 0.130860445 | 1.387048764 | 1.490762847 | 1.123575244 | 0 | 0 | 0 |
| miR-1181 | | | CCCGAGCCG | | | | | | | |
| hcmv- | MIMAT0003341 | 158 | CGACAUGGACGU | 0.309465166 | 0.527289196 | 0.622519192 | 0.463552136 | 0 | 0 | 0 |
| miR-US4 | | | GCAGGGGGAU | | | | | | | |
| hsa- | MIMAT0005929 | 159 | GUGGGGGAGAGG | 0.406783655 | -0.443832373 | -0.31163968 | 0.059486458 | 0.796595433 | 0.59115968 | -0.287389318 |
| miR-1275 | | | CUGUC | | | | | | | |
| hsa- | MIMAT0005898 | 160 | AAUGGAUUUUUG | 0.590126893 | 0 | 0 | 0 | 0 | 0 | 0 |
| miR-1246 | | | GAGCAGG | | | | | | | |
| hsa-miR- | MIMAT0003340 | 161 | UCGGGGAUCAUC | 0.664671956 | 0 | 0 | 0 | 0 | 0 | 0 |
| 542-5p | | | AUGUCACGAGA | | | | | | | |
| hsa-miR- | MIMAT0004603 | 162 | UCACAAGUCAGG | 1.015514337 | 1.683124035 | 1.613091961 | 1.524968419 | 0 | 0 | 0 |
| 125b-2* | | | CUCUUGGGAC | | | | | | | |
| hsa- | MIMAT0004951 | 163 | GUGAACGGGCGC | 1.096811763 | 0.846291383 | 0.727151257 | 0.008350384 | 0 | 0 | 0 |
| miR-887 | | | CAUCCCGAGG | | | | | | | |
| hsa- | MIMAT0005828 | 164 | CACUGUAGGUGA | 1.585755399 | 1.539217427 | 1.656479534 | 1.818070955 | -0.378264317 | -0.372494306 | 0.097899635 |
| miR-1183 | | | UGGUGAGAGUGG | | | | | | | |
| | | | GCA | | | | | | | |
| hsa- | MIMAT0005878 | 165 | UGCUGGAUCAGU | 2.33195002 | 2.878799542 | 3.01876665 | 2.468217674 | 0.143640016 | 0.172367393 | 0 |
| miR-1287 | | | GGUUCGAGUC | | | | | | | |
| hsa-miR- | MIMAT0002177 | 166 | UCCUGUACUGAG | 2.480223087 | 2.983018321 | 2.892730657 | 2.220008143 | 0 | 0 | 0 |
| 486-5p | | | CUGCCCCGAG | | | | | | | |
| hsa- | MIMAT0000442 | 167 | AUAAAGCUAGAU | 4.265651048 | 3.041134258 | 3.017380992 | 2.385982876 | 0 | 0 | 0 |
| miR-9* | | | AACCGAAAGU | | | | | | | |

1. A method for treating a subject having a cancer, the method comprising:
administering an epidermal growth factor receptor tyrosine kinase inhibitor (EGFR-TKI) agent to the subject; and
administering a synthetic oligonucleotide including the sequence of a microRNA selected from miR-34, miR-124, miR-126, miR-147, miR-215, and microRNAs listed in Appendix A as SEQ ID NOs:8-122 to the subject,
wherein if the EGFR-TKI agent is gefitinib, the microRNA is not miR-126.

2. The method of claim 1, wherein the EGFR-TKI agent is erlotinib.

3. The method of claim 1, wherein the cancer is lung cancer.

4. The method claim 3, wherein the lung cancer is non-small cell lung (NSCL) cancer.

5. The method of claim 1, wherein the cancer is resistant to treatment with the EGFR-TKI agent alone.

6. The method of claim 5, wherein the resistance is primary.

7. The methods of claim 5, wherein the resistance is secondary (acquired).

8. The method of claim 1, wherein the EGFR-TKI agent is administered at an effective dose that is at least 50% below the dose needed to be effective in the absence of the synthetic oligonucleotide administration.

9. The method of claim 1, wherein the IC₅₀ of the EGFR-TKI agent is reduced at least 2-fold relative to the IC₅₀ in the absence of the synthetic oligonucleotide administration.

10. The method of claim 1, wherein the cancer is liver cancer.

11. The method of claim 10, wherein the liver cancer is hepatocellular carcinoma (HCC).

12. The method of claim 1, wherein the subject has a KRAS mutation.

13. The method of claim 1, wherein the subject has an EGFR mutation.

14. The method of claim 1, wherein the cancer comprises a metastatic lesion in the liver.

15. The method of claim 1, wherein the EGFR-TKI agent comprises gefitinib, afatinib, panitumumab, or cetuximab.

16. The method of claim 4, wherein the NSCL has secondary resistance to treatment with the EGFR-TKI agent alone.

17. The method of claim 11, wherein the HCC has secondary resistance to treatment with the EGFR-TKI agent alone.

18. The method of claim 1, wherein the EGFR-TKI agent is erlotinib and the synthetic oligonucleotide comprises a sequence that is at least 80% identical to SEQ ID NO:1.

19. The method of claim 1, wherein the EGFR-TKI agent is a HER2 inhibitor.

20. The method of claim 19, wherein the HER2 inhibitor is lapatinib, pertuzumab, or trastuzumab.

21. The method of claim 1, wherein the EGFR-TKI agent is erlotinib, the synthetic oligonucleotide comprises a sequence that is at least 80% identical to SEQ ID NO:1, SEQ ID NO:168, or SEQ ID NO:169, and the cancer is non-small cell lung (NSCL).

22. The method of claim 1, wherein the EGFR-TKI agent is erlotinib, the synthetic oligonucleotide comprises a sequence that is at least 80% identical to SEQ ID NO:1, SEQ ID NO:168, or SEQ ID NO:169, and the cancer is pancreatic cancer.

23. The method of claim 1, wherein the EGFR-TKI agent is lapatinib, the synthetic oligonucleotide comprises a sequence that is at least 80% identical to SEQ ID NO:1, SEQ ID NO:168, or SEQ ID NO:169, and the cancer is breast cancer.

24. The method of claim 1, wherein the EGFR-TKI agent is afatinib, the synthetic oligonucleotide comprises a sequence that is at least 80% identical to SEQ ID NO:1, SEQ ID NO:168, or SEQ ID NO:169, and the cancer is non-small cell lung (NSCL).

25. A method for treating a subject having a cancer, the method comprising:
administering an epidermal growth factor receptor tyrosine kinase inhibitor (EGFR-TKI) agent to the subject; and
administering a synthetic oligonucleotide comprising a sequence that is at least 80% identical to SEQ ID NO:1-6, 8-122, or 168-179 to the subject,
wherein if the EGFR-TKI agent is gefitinib, the sequence is not identical to SEQ ID NO:3.

26. The method of claim 1, wherein the microRNA is miR-34.

27. A method for treating a subject having lung cancer, the method comprising:
administering an epidermal growth factor receptor tyrosine kinase inhibitor (EGFR-TKI) agent to the subject; and
administering a synthetic oligonucleotide mimic of miR-34a, miR-34b, or miR-34c to the subject.

28. The method claim 27, wherein the lung cancer is non-small cell lung (NSCL) cancer.

29. The method of claim 28, wherein the NSCL is resistant to treatment with the EGFR-TKI agent alone.

30. A method for treating a subject having liver cancer, the method comprising:
administering an epidermal growth factor receptor tyrosine kinase inhibitor (EGFR-TKI) agent to the subject; and
administering a synthetic oligonucleotide mimic of miR-34a, miR-34b, or miR-34c to the subject.

31. The method claim 30, wherein the liver cancer is hepatocellular carcinoma (HCC).

32. The method of claim 31, wherein the HCC is resistant to treatment with the EGFR-TKI agent alone.

33. A method for treating a subject having breast cancer, the method comprising:
administering an epidermal growth factor receptor tyrosine kinase inhibitor (EGFR-TKI) agent to the subject; and
administering a synthetic oligonucleotide mimic of miR-34a, miR-34b, or miR-34c to the subject.

34. The method of claim 33, wherein the NSCL is resistant to treatment with the EGFR-TKI agent alone.

35. The method claim 33, wherein the EGFR-TKI agent is lapatinib.
